(19) United States
(12) Patent Application Publication
(10) Pub. No.: US 2025/0129387 A1
(43) Pub. Date: Apr. 24, 2025

(54) METHODS FOR GENOME EDITING

(71) Applicant: Biotalys NV, Sint-Denijs-Westrem (BE)

(72) Inventors: Lucia Nancy COCONI LINARES, Sint-Denijs-Westrem (BE); Aleksandar DE BRUYCKER, Sint-Denijs-Westrem (BE); Alrik Pieter LOS, Sint-Denijs-Westrem (BE); Simon Hendrik B RYS, Sint-Denijs-Westrem (BE)

(21) Appl. No.: 18/834,104

(22) PCT Filed: Feb. 2, 2023
(86) PCT No.: PCT/EP2023/052604

§ 371 Date: Jul. 29, 2024

(30) Foreign Application Priority Data
Feb. 2, 2022 (EP) 22154861.3

(51) Int. Cl. C12N 15/90; C12N 9/22; C12N 15/11

(52) U.S. Cl. CPC C12N 15/902; C12N 9/22; C12N 15/111; C12N 2310/20

(57) ABSTRACT

The present invention relates to a method for editing the genome of a cell, such as a filamentous fungus cell. The method of the present invention requires the cell to be contacted with at least one pair of ribonucleoproteins, at least one donor-DNA construct and a selectable marker such that they are introduced into the cell. The present invention is especially suitable for multiplex genome editing of cells such as filamentous fungus cells. The current invention further relates to a composition, a cell obtainable by the method of the invention and a method for the production of a compound of interest. The present invention also relates to a method for high throughput transformation of a cell such as a filamentous fungus cell.

### FIELD OF THE INVENTION

The invention relates to the field of molecular biology and cell biology. More specifically, the invention relates to methods for genome editing of cells. The invention further relates to compositions, cells obtainable by the method of the invention and to methods for the production of a protein of interest. The invention further relates to high-throughput methods for transforming fungal cells.

### BACKGROUND

Different species of filamentous fungi have historically been used in fermentations and were selected by centuries of use. In more recent times, filamentous fungi are being used for their properties to produce extracellular plant biomass-degrading enzymes. This interesting aspect was mainly exploited with the production of biofuels as a goal. The key producers of extracellular (hemi)-cellulases are Aspergillus, Trichoderma, Penicillium and Neurospora species and over the past decades these strains have been improved using random mutagenesis, selection and genetic engineering with some species and strains now reported to produce up to 100 g/l of extra-cellular (hemi)cellulases (Cherry J R, Fidantsef A L, Opin. Biotechnol. 14(4), 438-443). Such protein production levels have spurred researchers to try and utilize filamentous fungi for the production of recombinant proteins by using strong endogenous promoters, signal peptides, and carrier (hemi)cellulolytic genes fused to the target genes. Very often however, these attempts did not produce the desired or hoped for expression levels of recombinant proteins. For example, during the production of a biological product, such as conventional monoclonal antibodies, unsatisfactory yields were reported ranging from 0.15 g/l in T. reesei to 0.9 g/l in A. niger. Such low amounts of biological product are insufficient for profitable production of proteins in industrial biotechnology, pharmacological and agricultural applications (Nyyssonen et al, 1993, Biotechnology. 11; Ward et al 2004, Appl. Environ. Microbiol. 70).

Many efforts have been undertaken to increase expression levels from filamentous fungi, such as searching for new promoters, deleting regulators such as catabolite repression modulators, introduction of chaperones, and so forth (Nevalainen, 2004, Handbook of fungal biotechnology). But despite all these previous and ongoing efforts, no substantial progress has yet been reported in the yields of recombinant protein production in fungal hosts (Nevalainen et al 2014, Front. Microbiol. 5:75).

A common stumbling block remains the ability of engineering the genomes of filamentous fungi which is often a time intensive procedure requiring extensive screening to identify suitable recombinant cells. Furthermore, when multiple deletions of modifications need to be made several iterations are required to reach the desired strain.

The so-called CRISPR (clustered regularly interspaced short palindromic repeats) Cas9 genome editing system originally isolated from Streptococcus pyogenes has been widely used as a tool to modify the genomes of a number of microorganisms as well as higher organisms. The programmable Cas9 enzyme has two RNA-guided DNA endonuclease domains capable of targeting specific genomic sequences. The system has been described extensively for editing genomes in a variety of eukaryotes [Doudna, J. A. and E. Charpentier, Genome editing. The new frontier of genome engineering with CRISPR-Cas9. Science, 2014. 346(6213): p. 1258096], human stem cells [Paquet, D., et al., Efficient introduction of specific homozygous and heterozygous mutations using CRISPR/Cas9. Nature, 2016. 533: p. 125-129], mouse zygotes [Inui, M., et al., Rapid generation of mouse models with defined point mutations by the CRISPR/Cas9 system. Sci Rep. 2014; 4:5396], pigs [Wang, K., et al., Efficient Generation of Orthologous Point Mutations in Pigs via CRISPR-assisted ssODN-mediated Homology-directed Repair. Mol Ther Nucleic Acids. 2016 November; 5(11): e396], E. coli [Jiang, W., et al., RNA-guided editing of bacterial genomes using CRISPR-Cas systems. Nat Biotechnol, 2013. 31(3): p. 233-9], yeast [DiCarlo, J. E., et al., Genome engineering in Saccharomyces cerevisiae using CRISPR-Cas systems. Nucleic Acids Res, 2013. 41(7): p. 4336-43, [Horwitz, A. A., et al., Efficient Multiplexed Integration of Synergistic Alleles and Metabolic Pathways in Yeasts via CRISPR-Cas. Cell Syst. 2015. 1(1): p. 88-96], Lactobacillus [Oh, J. H. and J. P. van Pijkeren, CRISPR-Cas9-assisted recombineering in Lactobacillus reuteri. Nucleic Acids Res, 2014. 42(17): p. e131] and filamentous fungi such as Trichoderma reesei [Liu, R., et al., Efficient genome editing in filamentous fungus Trichoderma reesei using the CRISPR/Cas9 system. Cell Discovery, 2015. 1].

The power of the Cas9 system lies in its simplicity and ability to target and edit a specific gene of interest. In addition, it is possible to target multiple genes for modification (multiplexing) in a single reaction, to generate insertions and deletions as well as silence or activate genes. In 2012 the Cas9 protein was shown to be a dual-RNA guided endonuclease protein [Jinek, M., et al., A programmable dual-RNA-guided DNA endonuclease in adaptive bacterial immunity. Science, 2012. 337(6096): p. 816-21.].

Further development has led to the engineering of a single guide-RNA molecule that guides the endonuclease to its DNA target. The single guide-RNA retains the critical features necessary for both interaction with the Cas9 protein and targeting the desired nucleotide sequence. When complexed with the RNA molecule, the Cas9 protein binds to the target sequence and creates a double stranded break using two catalytic domains.

When engineered to contain a single amino acid mutation in either catalytic domain, the Cas9 protein functions as a nickase, a variant protein with single stranded cleavage activity. Genome editing in Clostridium cellulolyticum via CRISPR-Cas9 nickase was recently demonstrated by Xu et al. [Xu, T., et al., Efficient Genome Editing in Clostridium cellulolyticum via CRISPR-Cas9 Nickase. Appl Environ Microbiol, 2015. 81(13): p. 4423-31.].

A plethora of scientific publications and published patent applications relating to genome editing has become available. More recently, a general method for transforming a replicative plasmid carrying the S. pyogenes Cas9-encoding gene into Aspergillus niger was described by Nødvig et al. [A CRISPR-Cas9 System for Genetic Engineering of Filamentous Fungi. 2015. PLoS ONE 10(7): e0133085. doi:10.1371/journal. pone.0133085] and more recently the transformation of a cas9 guide-RNA ribonucleoprotein to Aspergillus niger was reported by Zou et al. [Efficient genome editing in filamentous fungi via an improved CRISPR-Cas9 ribonucleoprotein method facilitated by chemical reagents. 2021. Microbial biotechnology: 14(6) pages 2343-2355].

Many new polynucleotide-guided and programmable endonucleases have been described since the first discovery of the Cas9 enzyme, including, for example, Cas12a (previously named Cpf1 (Makarova et al., 2017)). Cas12a is a class 2/type V RNA-guided endonuclease discovered in several bacterial genomes and one archaeal genome (Makarova et al., 2015) and filamentous fungi [Vanegas, K. G., Jarczynska, Z. D., Strucko, T., Mortensen, U. H., 2019. Cpf1 enables fast and efficient genome editing in Aspergilli. Fungal Biol. Biotechnol. 6, 1-10]. And a more recent example, is the MAD7 (also known as ErCas12a) enzyme isolated from Madagascar and described by Inscripta™, who released its DNA sequence and pledged on their website that the MAD7 enzyme is royalty-free for all R&D use. Even so, the MAD7 gene editing systems were recently patented by Inscripta™. MAD7 has been shown to be effective in both microbial and mammalian systems [Liu Z et al. ErCas12a CRISPR-MAD7 for Model Generation in Human Cells, Mice, and Rats. 2020. Cris J. 3:97-108].

Commonly, genome editing technology relies on DNA-based expression cassettes for delivering the Cas endonucleases and their guide-RNAs to the cell, these techniques increase off-target effects due to the continued presence of the cas endonucleases and guide-RNAs inside the cell and leads to possible random integration due to plasmids continuing to express Cas and Guide-RNAs into the cells. Likewise, the potential of this technology is limited to cells that are engineered to express cas endonucleases and guide-RNAs.

For efficient multiplex genome engineering, there is a need to improve construction of multiple guide nucleotide expression DNA constructs. As a default, CRISPR arrays would be chemically synthesized as linear dsDNA by commercial vendors. Unfortunately, the reoccurring repeat sequences inherent to these arrays currently pose major technical complications when assembling individually synthesized oligonucleotides, resulting in vendors regularly rejecting customer requests even for a minimal single-spacer array. Gene synthesis has offered a more reliable means of obtaining custom CRISPR arrays. However, synthesis often comes at large cost (˜5× the price of a linear dsDNA) and timeframes (˜1 month), and the synthesis can often fail. As an alternative to CRISPR arrays, the cas endonuclease can be complexed together with the guide RNA in vitro to form a Ribonucleoprotein or RNP after which the complex is transformed into the cell. This method has been used recently by Kuivanen et al 2019 [Kuivanen et al 2019. Development of microtiter plate scale CRISPR/Cas9 transformation method for Aspergillus niger based on in vitro assembled ribonucleoprotein complexes. Fungal Biology and Biotechnology: 6(3)] using Cas9 RNP in Aspergillus niger and Zou et al 2021 [Gen Zou et al. 2021. Efficient genome editing in filamentous fungi via an improved CRISPR-Cas9 ribonucleoprotein method facilitated by chemical reagents. Microbial biotechnology, 14(6):2343-2355] using Cas9 RNP in Trichoderma reesei, Coryceps militaris and Aspergillus Oryzae. It is often desirable to provide a donor DNA to repair the double strand break or breaks introduced by the RNP. This for example to ensure complete deletion of the target gene. These donor DNAs have 5′ and 3′ flanking regions that have sequence identity to the genome of the cell in order to serve as repair templates during homologous recombination repair. Typically, larger homology regions are used of over 1000 bp. Together with the need to include a selection marker in such a donor DNA, this leads to large nucleotide constructs of up to or over 5000 nt in length. In cases where multiple targets need to be targeted, these large donor DNA constructs become very costly and time consuming to construct. Furthermore, transformation of larger DNA constructs can become less efficient compared to smaller DNA fragments. Although, smaller 5′ and 3′ flanking regions of as low as 20 nt have been used, they would still need to flank a selection marker and when the objective is to provide a gene insertion a protein of interest would need to be flanked as well, which would still lead to rather large donor DNA constructs.

The current invention provides an improvement over current CRISPR based methods allowing for a more versatile and efficient way of performing one or multiple genetic modifications in filamentous fungi.

### SUMMARY OF THE INVENTION

The inventors have developed a method based on CRISPR-RNA-guided DNA endonuclease mediated cleavage of target DNA within a cell, such as a filamentous fungal cell. The method of the current invention allows for the marker-less editing of one or multiple targets simultaneously in the genetic material of a cell. Marker-less in this context indicates that, typically, no selectable marker need be introduced into the genome of a cell to be edited.

It was surprisingly found that cells comprising the all intended (multiple) edits may be generated with higher efficiencies than cells comprising none or only some of the intended edits.

The method thus provides a convenient and time efficient way of obtaining multiple edits in cell per round of transformation. Furthermore, by providing a selectable marker that is easily cured and does not integrate into the genetic material of the cell, multiple transformation rounds can follow each other without the need for intensive curing of the selectable marker from the cell (as is often required). Additionally, the absence of a selectable marker that needs to be integrated at the site of editing allows for a reduced size of donor-DNA fragments that are needed to repair the DNA breaks introduced by the endonucleases, hence greatly increasing the flexibility of the system.

Further provided is a high-throughput transformation method that may be used with the invention. Advantageously, the high-throughput method for transformation allows for transformations (such as transformations involved in the genome editing method of the invention) to be performed in micro-titer plates using low volumes (10 to 20 times less compared to a conventional method) of cells, nucleic acids, proteins and other reagents involved in transformation reactions. The high-throughput method further reduces the incubation times for performing a transformation. Together with the gene editing method, the high-throughput transformation method increases the speed and efficiency with which one or multiple gene edits can be made in a cell. Interestingly, the high-throughput method further increases the efficiency of obtaining successful genome edits.

Further provided are compositions and methods for the modification of microbial host cells, which may be suitable for providing microbial host cells suitable for the production of a compound of interest, in particular a recombinant protein.

According to the invention, there is thus provided a method for genome editing within a cell comprising contacting the cell with at least one pair of ribonucleoproteins such that the at least one pair of ribonucleoproteins are introduced into the cell, and whereby (i) the ribonucleoproteins are pre-assembled in vitro, and whereby (ii) each pair of ribonucleoproteins targets one locus in the cell; and further contacting the cell with at least one donor-DNA construct such that the at least one donor-DNA construct are introduced into the cell, wherein (i) the at least one donor-DNA construct has a 5′-end sequence which is at least partially complementary with the genome of the cell upstream of a first break in the genome of the cell, and where the first break is caused by the first of the pair of ribonucleoproteins, and wherein (ii) the at least one donor-DNA construct has a 3′-end sequence which is at least partially complementary with the genome of the cell downstream of a second break in the genome of the cell, and where the second break is caused by the second of the pair of ribonucleoproteins, and wherein (iii) the at least one donor-DNA construct serves as a template for the repair of the first and the second break by homologous recombination repair; and further contacting the cell with a selectable marker such that the selectable marker is introduced into the cell; and optionally, screening the cell for the genome edits introduced by the donor-DNA construct.

The invention also provides a composition comprising an RNA-guided DNA endonuclease and at least one pair of guide-RNAs, whereby (i) the endonuclease and the at least one pair of guide-RNAs are capable of assembling in vitro into at least one pair of ribonucleoproteins, and whereby (ii) each pair of ribonucleoproteins targets one locus in a cell; and, optionally, at least one donor-DNA construct wherein (i) the at least one donor-DNA construct has a 5′-end sequence which is at least partially complementary with the genome of the cell upstream of the target sequence of the first of the pair of ribonucleoproteins, and wherein (ii) the at least one donor-DNA construct has a 3′-end sequence which is at least partially complementary with the genome of the cell downstream of the target sequence of the second of the pair of ribonucleoproteins, and wherein (iii) the at least one donor-DNA construct serves as a template for homologous recombination repair of a first and a second break in the genome of the cell, wherein the first and second breaks are caused by the first and second of the pair of ribonucleoproteins, respectively; and a selectable marker.

The invention further provides a cell obtainable by the method of the invention, and a method for the production of a protein of interest comprising providing a cell obtained by the method of the invention capable of expressing the protein of interest, and cultivating the cell under conditions suitable for expressing the protein of interest, and optionally isolating the protein of interest.

In addition, the invention provides a high-throughput method for transforming fungal cells comprising the steps of: (i) providing fungal protoplasts, and (ii) mixing the protoplasts with a nucleic acid construct, and (iii) further mixing the protoplasts with a polyethylene glycol solution, (iv) incubating the protoplasts and subsequently adding an additional solution of polyethylene glycol to the protoplasts mixture (v) resuspending the protoplasts and plating and selecting the antibiotic resistant colonies (vi) screening the cell for the genome edits introduced in step (ii).

### BRIEF DESCRIPTION OF THE DRAWINGS

FIG. 1 provides a schematic representation of the invention and examples of how the invention may be used.

FIG. 2 sets out the self-replicating episomal plasmid pAMA1-HygB.

FIG. 3 sets out the self-replicating episomal plasmid pAMA1-BleoR.

### DESCRIPTION OF THE SEQUENCE LISTING

SEQ ID NOs: 1 to 5 are the sequence of VHH-1, where SEQ ID NO: 1 is the full length sequence of VHH-1, SEQ ID NO: 2 is the full length sequence of VHH-1 but in which the first residue is changed to a Q residue, SEQ ID NO: 3 is the CDR1 of VHH-1, SEQ ID NO: 4 is the CDR2 of VHH-1 and SEQ ID NO: 5 is the CDR3 of VHH-1.

SEQ ID NOs: 6 to 9 and 14 are the sequences of VHH-2, where SEQ ID NO: 6 is the full length sequence of VHH-1, SEQ ID NO: 14 is the full length sequence of VHH-2 but in which the first residue is changed to a D residue, SEQ ID NO: 7 is the CDR1 of VHH-2, SEQ ID NO: 8 is the CDR2 of VHH-2 and SEQ ID NO: 9 is the CDR3 of VHH-2.

SEQ ID NOs: 10 to 13 and 15 are the sequences of VHH-3, where SEQ ID NO: 10 is the full length sequence of VHH-1, SEQ ID NO: 15 is the full length sequence of VHH-3 but in which the first residue is changed to a D residue, SEQ ID NO: 11 is the CDR1 of VHH-3, SEQ ID NO: 12 is the CDR2 of VHH-3 and SEQ ID NO: 13 is the CDR3 of VHH-3.

SEQ ID NO: 16 is the complete nucleotide sequence of the pAMA1-HygB plasmid.

SEQ ID NO: 17 is the complete nucleotide sequence of the pAMA1-BleoR plasmid.

SEQ ID NO: 18 is the nucleotide sequence of AMA1.

SEQ ID NOs: 19-20 are the guide-RNAs for the pair of RNPs for targeting the Trichoderma reesei are1 gene.

SEQ ID NOs: 21-22 are the guide-RNAs for the pair of RNPs for targeting the Trichoderma reesei cbh1 operon.

SEQ ID NOs: 23-24 are the guide-RNAs for the pair of RNPs for targeting the Trichoderma reesei pep5 gene.

SEQ ID NOs: 25-26 are the guide-RNAs for the pair of RNPs for targeting the Trichoderma reesei sep1 gene.

SEQ ID NOs: 27-28 are the guide-RNAs for the pair of RNPs for targeting the Trichoderma reesei gap2 gene.

SEQ ID NOs: 29-30 are the guide-RNAs for the pair of RNPs for targeting the Trichoderma reesei pep1 gene.

SEQ ID NOs: 31-32 are the guide-RNAs for the pair of RNPs for targeting the Trichoderma reesei gap1 gene.

SEQ ID NO: 33 is the donor-DNA construct for deleting the are1 gene of Trichoderma reesei.

SEQ ID NO: 34 is the donor-DNA construct for inserting the VHH-1 Q1D expression cassette into the cbh1 locus of Trichoderma reesei.

SEQ ID NO: 35 is the donor-DNA construct for deleting the pep5 gene of Trichoderma reesei.

SEQ ID NO: 36 is the donor-DNA construct for deleting the sep1 gene of Trichoderma reesei.

SEQ ID NO: 37 is the donor-DNA construct for deleting the gap2 gene of Trichoderma reesei.

SEQ ID NO: 38 is the donor-DNA construct for deleting the pep1 gene of Trichoderma reesei.

SEQ ID NO: 39 is the donor-DNA construct for deleting the gap1 gene of Trichoderma reesei.

SEQ ID NO: 40 and 41 are the PCR primer pair for amplifying the donor DNA for the deletion of gap1 flanked by approximately 50 bp long 5′-end and 3′-end sequences.

SEQ ID NO: 42 and 43 are the PCR primer pair for amplifying the donor DNA for the deletion of gap1 flanked by approximately 100 bp long 5′-end and 3′-end sequences.

SEQ ID NO: 44 and 45 are the PCR primer pair for amplifying the donor DNA for the deletion of gap1 flanked by approximately 200 bp long 5′-end and 3′-end sequences.

SEQ ID NO: 46 and 47 are the PCR primer pair for amplifying the donor DNA for the deletion of gap1 flanked by approximately 350 bp long 5′-end and 3′-end sequences.

SEQ ID NO: 48 and 49 are the PCR primer pair for amplifying the donor DNA for the deletion of gap1 flanked by approximately 700 bp long 5′-end and 3′-end sequences.

SEQ ID NO: 50 and 51 are the PCR primer pair for amplifying the donor DNA for the deletion of slp1 flanked by approximately 50 bp long 5′-end and 3′-end sequences.

SEQ ID NO: 52 and 53 are the PCR primer pair for amplifying the donor DNA for the deletion of slp1 flanked by approximately 100 bp long 5′-end and 3′-end sequences.

SEQ ID NO: 54 and 55 are the PCR primer pair for amplifying the donor DNA for the deletion of slp1 flanked by approximately 200 bp long 5′-end and 3′-end sequences.

SEQ ID NO: 56 and 57 are the PCR primer pair for amplifying the donor DNA for the deletion of slp1 flanked by approximately 350 bp long 5′-end and 3′-end sequences.

SEQ ID NO: 58 and 59 are the PCR primer pair for amplifying the donor DNA for the deletion of slp1 flanked by approximately 700 bp long 5′-end and 3′-end sequences.

The sequences are set out in Table 5.

### DETAILED DESCRIPTION OF THE INVENTION

Reference to any prior art in this specification is not, and should not be taken as, an acknowledgment or any form of suggestion that this prior art forms part of the common general knowledge in any country.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs.

The present invention will be described with respect to particular embodiments but the invention is not limited thereto but only by the claims. Any reference signs in the claims shall not be construed as limiting the scope.

Where the term “comprising” is used in the present description and claims, it does not exclude other elements or steps.

Where an indefinite or definite article is used when referring to a singular noun e.g. “a” or “an”, “the”, this includes a plural of that noun unless something else is specifically stated.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art.

The definitions provided herein should not be construed to have a scope less than understood by a person of ordinary skill in the art. Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The present invention relates to a method for genome editing a cell, such as a filamentous fungal cell. In a preferred embodiment the cell is contacted with at least one pair of ribonucleoproteins (RNPs) such that the at least one pair of ribonucleoproteins are introduced into the cell, whereby the ribonucleoproteins are pre-assembled in vitro, and whereby each pair of ribonucleoproteins targets one locus in the cell. Hence, the method of the invention has as an objective to modify, for example delete, certain loci, such as an open reading frame, within the genetic material present in the cell.

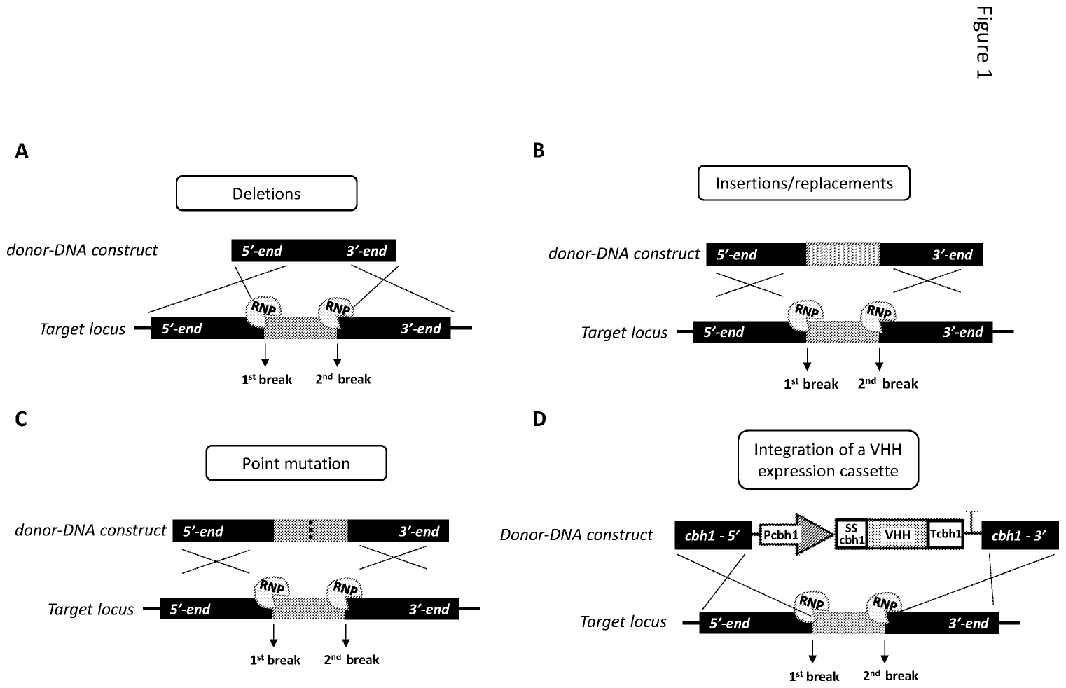

The phrase “pair of ribonucleoproteins” refers to a first ribonucleoprotein and a second ribonucleoprotein for targeting a single locus in the host cell. The first ribonucleoprotein comprises an RNA-guided DNA endonuclease and a guide RNA that guides the endonuclease to introduce a first break in the genome of the cell at the 5′ end of the target locus. The second ribonucleoprotein comprises an RNA-guided DNA endonuclease and a guide RNA, different to the guide RNA in the first ribonucleoprotein, that guides the endonuclease to introduce a second break in the genome of the cell at the 3′ end of the target locus (see FIG. 1).

Thus, the at least one pair of ribonucleoproteins serves to introduce targeted double strand breaks at specified locations or target sequences within the cell so that the double stand breaks might serve as a starting point for the cellular homologous recombination repair system. The specified locations where double strand breaks are to be introduced in the genome of the cell are specified by designing each guide RNA such that the corresponding RNP will cut the genomic DNA at the region complementary to the guide RNA and as further defined by the features of the CRISPR system used. The skilled person will know how to design the guide RNAs such that the double strand breaks occur with a very high probability in these sites where they were intended. It may be possible to carry out the invention wherein the at least one pair of ribonucleoproteins introduce targeted single strand breaks.

In a preferred embodiment the cell is further contacted with at least one donor-DNA construct that, when it is introduced into the cell together with the at least one pair of RNPs can serve as a template for the repair of the double strand breaks produced by the at least one pair of RNPs. Therefore, the at least one donor-DNA construct has a 5′-end sequence which is complementary, or is at least partially complementary, to the genome of the cell upstream of a first break in the genome of the cell caused by the first of the pair of ribonucleoproteins, and wherein the at least one donor-DNA construct has a 3′-end sequence which is complementary, or is at least partially complementary, to the genome of the cell downstream of a second break in the genome of the cell and where the second break is caused by the second of the pair of ribonucleoproteins (see FIG. 1). It follows that the at least one donor-DNA construct can serve as a template for the repair of the first and the second break by homologous recombination repair. As such, the donor-DNA construct can be used to introduce genetic changes or edits or modifications into the cell. For instance, where the 5′-end and the 3′-end sequences of the donor-DNA construct are complementary to the genome of the cell upstream and downstream of the first and second break caused by the at least one pair of ribonucleoproteins (respectively), and where the donor-DNA does not contain additional nucleotides, this would result in a clean (i.e. no extra genetic material such as an antibiotic resistance cassette are introduced, FIG. 1 A) excision or deletion of the locus or genetic material present between the two breaks caused by the at least one pair of RNPs. The donor-DNA construct can be introduced either as double-stranded or single-stranded DNA.

In some embodiments the 5′-end and the 3′-end sequences of the donor-DNA construct may be similar or essentially identical in length. In other embodiments the 5′-end and 3′-end sequences can have different lengths. The 5′-end and/or 3′-end of the donor-DNA constructs can be between 20 nucleotides and 2000 nucleotides in length. In some embodiments the 5′-end and/or 3′-end sequences of the donor-DNA constructs can be around 20 nucleotides or more, around 30 nucleotides or more, around 40 nucleotides or more, around 50 nucleotides or more, around 100 nucleotides or more, around 200 nucleotides or more, around 400 nucleotides or more around 600 nucleotides or more, around 800 nucleotides or more, around 1000 nucleotides or more, around 1500 nucleotides or more or around 2000 nucleotides or more. In practice, shorter lengths are preferred, such as between 20 and 100 nucleotides, however the skilled person will appreciate that a tradeoff might exist between efficiencies with which the donor-DNA construct is introduced into the target region and the length of the 5′-end and 3′-end sequences of the donor-DNA, where longer 5′- and 3′-end sequences might lead to increased efficiency in homologous recombination. On the other hand the total length of the donor-DNA construct cannot become too long, since larger donor-DNA constructs (for example over 5000 nucleotides or higher) will lead to reduced efficiencies of the donor-DNA construct being integrated. Without wanting to be bound by theory, larger donor-DNA constructs might have reduced efficiencies of entering the cell and once entered in the cell might have a reduced efficiency of reaching the target loci and might thus not reach the homologous recombination machinery efficiently. Therefore, the skilled person will appreciate that the tradeoff exists between keeping the length of the donor-DNA construct as short as possible while maximizing the length of the 5′ and 3′-end sequences.

In the preferred embodiment the cell is further contacted with a selectable marker such that the selectable marker is introduced into the cell alongside the at least one pair of RNPs and at least one donor DNA. Preferably, the donor-DNA does not comprise the selectable marker. The further introduction of a selectable marker allows for the selection of those cells that have incorporated the selectable marker, which facilitates the further screening of cells for genome edits introduced by the donor-DNA by reducing the number of cells that need to be screened. Optionally, the cell is then screened for the genome edits introduced by the donor-DNA.

In one embodiment, the selectable marker as described above is introduced into the cell in such a way that the resulting mother and daughter cells are able to survive and proliferate on or in a growth medium containing the selective pressure to which the selectable marker confers resistance (for example an antibiotic that would otherwise be lethal to the cells lacking the selectable marker containing a corresponding antibiotic resistance cassette). This may be achieved in several ways such as a conventional approach where the selectable marker is provided in the donor-DNA construct by flanking the selectable marker with 5′ and 3′-end sequences of the donor-DNA. More preferably, the selectable marker may be provided for example in a plasmid unable to replicate in the cell (also referred to as a suicide plasmid) but that is capable of inserting into the genome of the cell for example by homologous recombination repair or by site specific integration (such as by using FLP-FRT recombination or Cre-Lox recombination).

The applicant has surprisingly found that when a cell is contacted with at least one pair of RNPs, at least one donor-DNA construct and a separate selectable marker, those cells that can proliferate on or in growth medium containing a selective pressure (because of the successful uptake of the selectable marker) also have a surprisingly high probability of having incorporated the at least one pair of RNPs and the at least one donor-DNA construct, even when the selectable marker is not comprised within the donor-DNA (i.e. where the 5′-end and 3′-end sequences of the donor-DNA construct flank the selectable marker). Hence, when screening the cell for the genome edits introduced by the donor-DNA construct, a high probability of success was observed requiring minimal screening efforts. Importantly, by not requiring the donor-DNA construct to include a selectable marker, the donor-DNA construct can have a significant reduction in size (i.e. the size in nucleotides that would be required for expressing, for example, an antibiotic resistance marker (such as hygB or bleoR) which greatly benefits the recombination efficiencies for reasons that were theorized above. Furthermore, by removing the selectable marker from the donor-DNA construct, alternative genetic constructs can be introduced into the donor-DNA construct. For example, the donor-DNA construct can be designed such that it includes an expression cassette expressing a protein of interest.

In a further, more preferred embodiment, the selectable marker as described above is contained in a self-replicating episomal plasmid. As such the selectable marker does not need to be inserted into the genome of the cell, rather it can exist independently and replicate in order to be maintained in the daughter cells originating from the first mother cell that was contacted by the at least one pair of RNPs and the at least one donor-DNA construct such as described above. This has the advantage that the selectable marker can be removed relatively easy from the genome edited cell provided by the method of the invention, by growing the genome edited cells for several generations on growth media lacking the selective pressure until cells can be found that have lost the self-replicating episomal plasmid by random genetic drift or segregational drift.

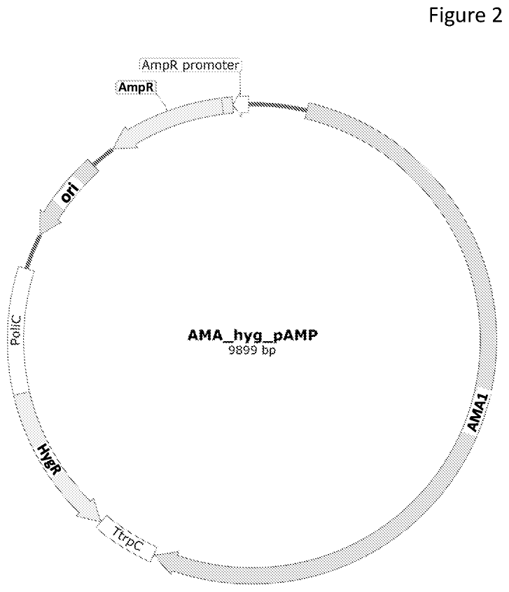

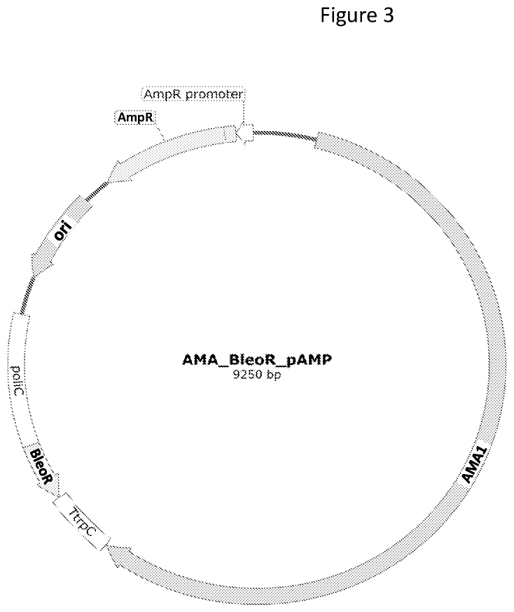

In an even more preferred embodiment, the self-replication episomal plasmid may be an AMA1-based plasmid [Eleksenko et al., The plasmid replicator AMA1 in Aspergillus nidulans is an inverted duplication of a low-copy-number dispersed genomic repeat, 1996, Mol. Micro, 19(03): 565-574]. With AMA1-based plasmid it is meant a plasmid or vector containing the approximately 5.5 kb AMA1 sequence (SEQ ID NO: 18) or a functional variant thereof i.e. the ability to promote extrachromosomal plasmid replication at low-copy numbers. The applicant has found that AMA1-based plasmids are ideal for use in the method of the invention since they may be lost from the genome edited cells easily when grown in the absence of selective pressure. Here are provided two examples of AMA1-based plasmids suitable for use in the invention: pAMA1-hygB comprising a hygromycin expression cassette and providing resistance against the antibiotic hygromycin (SEQ ID NO: 16 and FIG. 2) and pAMA1-bleoR comprising a bleomycin resistance cassette and providing resistance against the antibiotic bleomycin (SEQ ID NO: 17 and FIG. 3).

In a further embodiment the pAMA1-hygB and pAMA1-bleoR may be used in conjunction when several rounds of genome editing according to the method of the invention are envisioned. For example, a first set of one or more genome edits may be constructed by contacting the cell with at least one pair of ribonucleoproteins and at least one donor-DNA construct and for example pAMA1-hygB self-replicating episomal plasmid. A second round according to the method of the invention can thereafter be started and where one or more additional genome edits may be constructed according to the method of the invention of the current embodiment but where pAMA1-hygB is replaced by pAMA-bleoR. Thereafter, a third round can be started recycling the first pAMA1-hygB plasmid and so on. Since pAMA1 plasmids are lost quickly in the absence of a selective pressure, no additional cultivation steps only to remove the selection marker are required between different rounds of genome editing, greatly speeding up the process of iteratively editing the genome of a cell by the recycling of selection markers. The person skilled in the art will recognize that next to hygB and bleoR alternative antibiotics suitable for the cell being edited in its genome can be used without this changing the expected outcome of the method of the invention. Furthermore, it follows that different combinations of pAMA1 based plasmids each harboring a different antibiotic resistance marker can be employed when recycling selection markers.

In a preferred embodiment, the method of the invention may be used to introduce multiple genetic edits at once in the cell. More specifically, by contacting the cell with multiple pairs of RNPs where each pair of RNP targets a different locus in the genome of the cell, multiple loci can be targeted simultaneously. The cell is further contacted with multiple corresponding donor-DNA constructs where each donor-DNA construct is constructed so as to serve as a template for homologous recombination for each of the set of double strands breaks caused by each pair of RNPs. In this preferred embodiment the cell is further contacted with a selectable marker such that it is introduced in the cell together with the multiple pairs of RNPs and the multiple corresponding donor-DNA constructs. The applicant has surprisingly found that when selecting for the presence of the selectable marker, a significant percentage of cells contain all the genome edits targeted by the multiple pairs of RNPs and the corresponding donor-DNA constructs. Hence, using minimal screening efforts the skilled person will readily identify those cells that contain all the required genome edits.

In a preferred embodiment, the method of the invention may be used to introduce multiple genetic edits at once in the cell. Thus, the method of the invention may comprise
a. contacting the cell with two pairs of ribonucleoproteins, a first and second pair of ribonucleoproteins, such that the two pairs of ribonucleoproteins are introduced into the cell
   i. whereby the ribonucleoproteins are pre-assembled in vitro, and
   ii. whereby each pair of ribonucleoproteins targets one (i.e., a different) locus in the cell; and
b. further contacting the cell with two donor-DNA constructs, a first and second donor-DNA construct, such that the two donor-DNA constructs are introduced into the cell
   i. wherein the first donor-DNA construct has a 5′-end sequence which is at least partially complementary with the genome of the cell upstream of a first break in the genome of the cell,
      where the first break is caused by the first of the first pair of ribonucleoproteins, and
   ii. wherein the first donor-DNA construct has a 3′-end sequence which is at least partially complementary with the genome of the cell downstream of a second break in the genome of the cell,
      where the second break is caused by the second of the first pair of ribonucleoproteins, and
   iii. wherein the first donor-DNA construct serves as a template for the repair of the first and the second break by homologous recombination repair; and
   iv. wherein the second donor-DNA construct has a 5′-end sequence which is at least partially complementary with the genome of the cell upstream of a third break in the genome of the cell,
      where the third break is caused by the first of the second pair of ribonucleoproteins, and
   v. wherein the second donor-DNA construct has a 3′-end sequence which is at least partially complementary with the genome of the cell downstream of a fourth break in the genome of the cell,
      where the fourth break is caused by the second of the second pair of ribonucleoproteins, and
   vi. wherein the second donor-DNA construct serves as a template for the repair of the third and the fourth break by homologous recombination repair; and
c. further contacting the cell with a selectable marker such that the selectable marker is introduced into the cell; and optionally, screening the cell for the genome edits introduced by the donor-DNA construct. Furthermore, the method of the invention may comprise
a. contacting the cell with three pairs of ribonucleoproteins, a first, second and third pair of ribonucleoproteins, such that the three pairs of ribonucleoproteins are introduced into the cell
   i. whereby the ribonucleoproteins are pre-assembled in vitro, and
   ii. whereby each pair of ribonucleoproteins targets one (i.e., a different) locus in the cell; and
b. further contacting the cell with three donor-DNA constructs, a first, second and third donor-DNA construct, such that the three donor-DNA constructs are introduced into the cell
   i. wherein the first donor-DNA construct has a 5′-end sequence which is at least partially complementary with the genome of the cell upstream of a first break in the genome of the cell,
      where the first break is caused by the first of the first pair of ribonucleoproteins, and
   ii. wherein the first donor-DNA construct has a 3′-end sequence which is at least partially complementary with the genome of the cell downstream of a second break in the genome of the cell,
      where the second break is caused by the second of the first pair of ribonucleoproteins, and
   iii. wherein the first donor-DNA construct serves as a template for the repair of the first and the second break by homologous recombination repair; and
   iv. wherein the second donor-DNA construct has a 5′-end sequence which is at least partially complementary with the genome of the cell upstream of a third break in the genome of the cell,
      where the third break is caused by the first of the second pair of ribonucleoproteins, and
   v. wherein the second donor-DNA construct has a 3′-end sequence which is at least partially complementary with the genome of the cell downstream of a fourth break in the genome of the cell,
      where the fourth break is caused by the second of the second pair of ribonucleoproteins, and
   vi. wherein the second donor-DNA construct serves as a template for the repair of the third and the fourth break by homologous recombination repair; and
   vii. wherein the third donor-DNA construct has a 5′-end sequence which is at least partially complementary with the genome of the cell upstream of a fifth break in the genome of the cell,
      where the fifth break is caused by the first of the third pair of ribonucleoproteins, and
   viii. wherein the third donor-DNA construct has a 3′-end sequence which is at least partially complementary with the genome of the cell downstream of a sixth break in the genome of the cell,
      where the sixth break is caused by the second of the third pair of ribonucleoproteins, and
   ix. wherein the third donor-DNA construct serves as a template for the repair of the fifth and the sixth break by homologous recombination repair; and
c. further contacting the cell with a selectable marker such that the selectable marker is introduced into the cell; and

optionally, screening the cell for the genome edits introduced by the donor-DNA construct.

As the skilled person will appreciate, the above can be extrapolated to methods where four or more genome edits are introduced into the cell, for example by using four or more pairs of ribonucleoproteins and for or more corresponding donor-DNA constructs.

Thus, the method of the invention may be used to introduced 2 or more genetic edits at once in the cell, 3 or more genetic edits at once in the cell, 4 or more genetic edits at once in the cell, 5 or more genetic edits per cell or 6 or more genetic edits per cell. Therefore, according to the method of the invention, the cell may be contacted with 2 or more pairs of RNPs, 3 or more pairs of RNPs, 4 or more pairs of RNPs, 5 or more pairs of RNPs or 6 or more pairs of RNPs, together with, respectively, 2 or more corresponding donor-DNA constructs, 3 or more corresponding donor-DNA constructs, 4 or more corresponding donor-DNA construct, 5 or more corresponding donor-DNA construct or 6 more corresponding donor-DNA construct, such that the 2 or more pairs of RNPs, 3 or more pairs of RNPs, 4 or more pairs of RNPs, 5 or more pairs of RNPs or 6 or more pairs of RNPs, and the, respectively, 2 or more corresponding donor-DNA constructs, 3 or more corresponding donor-DNA constructs, 4 or more corresponding donor-DNA constructs, 5 or more corresponding donor-DNA constructs or 6 more corresponding donor-DNA constructs are introduced into the cell.

In another embodiment of the invention the one or more donor-DNA construct comprises an additional nucleotide sequence inserted into the genome of the cell, at the targeted locus, by the homologous recombination repair. That is to say, next to the 5′-end and 3′-end sequences of the donor-DNA, which serve as a donor for homologous recombination repair of the double strand breaks introduced by each pair of RNPs, the donor-DNA construct may include an additional nucleotide sequence that is flanked by said 5′-end and 3′-end sequences. The additional nucleotide sequence may include a selectable marker. However, in some embodiments the additional nucleotide sequence does not include a selectable marker, where the selectable marker is already provided in a separate genetic construct (such as for example on a self-replicating episomal plasmid) that is contacted with the cell such that it is introduced in the cell.

In a further embodiment of the invention the additional nucleotide sequence may comprise a nucleotide sequence encoding a protein of interest. The additional nucleotide sequence may comprise a nucleotide sequence merely encoding a protein of interest (i.e. merely having the coding region of the protein of interest), but not capable of expressing a protein of interest (i.e. lacking for instance a promoter, or other genetic parts necessary for driving the expression of a protein of interest such as a Kozak sequence or ribosome binding site). In such an embodiment it is the objective to insert the nucleotide sequence encoding a protein of interest directly downstream or under the control of a native promoter of the cell. The nucleotide sequence encoding a protein of interest may also be placed inside a native operon (i.e. where a single promoter drives the expressing of one or more native proteins) that is controlled by a native promoter without interfering with the expression of the native protein or proteins of said native operon. In other embodiments the nucleotide sequence encoding a protein of interest may be inserted in frame into the coding region of a native protein. This would then lead to a fusion protein where the protein of interest will be coupled with a native protein or part of a native protein. As a specific illustrative example, the nucleotide sequence encoding a protein of interest may be inserted in frame and just downstream of the native nucleotide sequence encoding the cbhl signal peptide in Trichoderma whilst removing or deleting the remainder of the nucleotide sequence encoding the cbhl protein. This would then lead to a protein of interest expressed under control of the cbhl promoter and where the further secretion of the protein of interest is further controlled by the cbhl signal peptide.

In a further embodiment of the invention, the additional nucleotide sequence comprises a nucleotide sequence capable of expressing a protein of interest. That is to say, there is provided all the genetic elements necessary to express a protein of interest, such as a promoter, a kozak site or a ribosome binding site, a start and a stop codon and optionally a terminator sequence (see FIG. 1D for an example). In this embodiment, the nucleotide sequence capable of expressing a protein of interest can be inserted in any region of the genome of the cell provided it does not interfere with the proper functioning of the cell or where such a region would interfere with the proper expression of the protein of interest.

In another embodiment of the invention multiple donor-DNA constructs are provided with an additional nucleotide sequence. The multiple donor-DNA constructs may differ only in their 5′-end and 3′-end sequences (such that they target different loci in the cell genome), where the additional nucleotide sequence remains the same or essentially the same. As such, multiple copies of additional nucleotide sequences that are the same or essentially the same can be inserted in multiple loci on the genome of the cell. Alternatively, the multiple donor-DNA constructs may differ in their 5′-end and 3′-end sequences and they may differ in the additional nucleotide sequence. As such, multiple copies of different additional nucleotide sequences may be inserted in multiple loci on the genome of the cell. For instance, multiple donor-DNA constructs may comprise different nucleotide sequences capable of expressing different proteins of interest.

In a preferred embodiment, the method of the invention may be used to introduce multiple donor-DNA constructs that are provided with an additional nucleotide. Thus, the method of the invention may be used to introduce 2 or more additional nucleotide sequences at once in the cell, 3 or more additional nucleotide sequences at once in the cell, 4 or more additional nucleotide sequences at once in the cell, 5 or additional nucleotide sequences per cell or 6 or more additional nucleotides sequence per cell. Therefore, according to the method of the invention, the cell may be contacted with 2 or more pairs of RNPs, 3 or more pairs of RNPs, 4 or more pairs of RNPs, 5 or more pairs of RNPs or 6 or more pairs of RNPs, together with, respectively, 2 or more corresponding donor-DNA constructs providing additional nucleotide sequences, 3 or more corresponding donor-DNA constructs providing additional nucleotide sequences, 4 or more corresponding donor-DNA constructs providing additional nucleotide sequences, 5 or more corresponding donor-DNA constructs providing additional nucleotide sequences or 6 more corresponding donor-DNA constructs providing additional nucleotide sequences, such that the 2 or more pairs of RNPs, 3 or more pairs of RNPs, 4 or more pairs of RNPs, 5 or more pairs of RNPs or 6 or more pairs of RNPs, and the, respectively, 2 or more corresponding donor-DNA constructs providing additional nucleotide sequences, 3 or more corresponding donor-DNA constructs providing additional nucleotide sequences, 4 or more corresponding donor-DNA constructs providing additional nucleotide sequences, 5 or more corresponding donor-DNA constructs providing additional nucleotide sequences or 6 more corresponding donor-DNA constructs providing additional nucleotide sequences are introduced into the cell. Preferably, where two or more pairs of RNPs are introduced into the cell (as well as the two or more corresponding donor-DNA constructs), only a single selectable marker is introduced into the cell. Advantageously, this permits multiple different genome edits to be performed in a single transformation step.

In a further embodiment the additional nucleotide sequence in the at least one donor-DNA construct may comprise a nucleotide sequence that is native to the cell. As an illustrative example, a cell might be constructed where a protein of interest is a protein native of said cell and whereby it is preferred to introduce multiple copies of said native protein of interest into said cell, to, for example, enhance expression of the native protein.

In a further embodiment of the invention the additional nucleotide sequence provided by the donor-DNA construct may comprise a nucleotide sequence that aids to prevent polar effects when removing or deleting a locus in the genome of the cell. Polar effects occur when the deletion of a certain open reading frame allows the transcription and translational machinery of the cell to read through the deleted region and express a protein or multiple proteins downstream of the deleted regions and where this expression leads to unwanted side effects. Polar effects may also occur when the deletion of a certain open reading frame prevents the transcription and translational machinery of the cell to read through the deleted region and prevent expression of a protein or multiple proteins downstream of the deleted region and where this expression leads to unwanted side effects. For example, the additional nucleotide sequence might comprise a stop codon or a double stop codon to prevent the translational machinery to continue beyond the point of the deletion. The additional nucleotide sequence might comprise a set of 3 stop codons that are organized such that each of the stop codons has a different reading frame i.e. irrespective of the start codon used further upstream, the translational machinery will encounter a stop codon that is in frame with the polypeptide being constructed.

In a further embodiment of the invention the additional nucleotide sequence provided by the donor-DNA construct may comprise a nucleotide sequence that may serve other purposes for cellular engineering such as an frt or cre site to provide for points for site-specific recombination in further downstream applications.

In a further embodiment of the invention the additional nucleotide sequence provided by the donor-DNA construct may comprise specific nucleotides or a set of nucleotides that introduce indel mutations such as frameshift mutations, or mutations altering the amino acid encoded by a certain codon.

The current invention provides ways to quickly introduce genomic edits in a cell facilitated by RNA-guided DNA endonuclease based cleavage of target DNA. With the method of the invention multiple genome edits can be constructed simultaneously and when the method of the invention is combined with the use of a selectable marker encoded on a self-replicating plasmid that is easily cured from the cell lines (such as AMA1 based plasmids), the method of the invention can be repeated quickly with few cultivation steps. Crucially the possibility of introducing multiple genome edits at once saves the time that is usually needed to grow the cells, treat the cells so that they are amenable for receiving DNA (for example providing protoplasts of filamentous fungal cells), plating the cell on selective media, cultivating the cells, storing the cells and screening the cells for the correct genome edits. A cell comprising for example twelve genome edits may be constructed in just two cycles instead of the commonly needed 12 cycles of propagating, preparing, storing and screening of the cells to reach such a high number of genome edits. Furthermore, because the selectable marker can be introduced on a genetic construct that is not the donor-DNA construct, very small donor-DNA constructs can be made. When a deletion or small insertion or indel is the objective, the donor-DNA constructs can be as small as 1000 nucleotides or smaller, for example the donor-DNA constructs can be as small as 500 nucleotides or smaller, 100 nucleotides or smaller, or even 50 nucleotides or smaller. This allows for the donor-DNA constructs to be simply synthesized such as a standard PCR primer would be synthesized which provides a very fast and cost-effective way of providing donor-DNA constructs (unlike conventional methods that required multiple cloning steps and further PCR steps to provide a donor-DNA construct). Additionally, because the selectable marker can be introduced on a genetic construct that is not the donor-DNA construct, it becomes straightforward to cure the cell of the selectable marker. Moreover, when multiple genome edits are produced simultaneously, only one genetic marker needs to be cleared from the cell because, a single selectable marker can be used for multiple simultaneous genome edits (unlike conventional methods that would require for each genome edit that the introduced selectable marker is removed). Even more, where the selectable marker is provided on a self-replicating episomal plasmid, such as an AMA1-based plasmids (SEQ ID NO: 16 and SEQ ID NO: 17), no additional steps other than a very low number of cultivation steps (for instance 2 or 3 cultivation steps) need to be performed to clear the cell from the selectable marker and where multiple rounds of genome editing are needed (i.e. where the method of the invention is performed 2 or more times), no extra cultivation steps to remove the AMA1-based plasmid is needed in between methods since an alternative AMA1-based plasmid can be used while the cell is cured from the first AMA1-based plasmid.

The invention also provides a composition suitable for use in the methods of the invention. In this regard, examples of ribonucleoproteins, endonucleases, donor-DNA constructs etc. provided in the context of the method of the invention, equally apply to the composition of the invention mutatis mutandis. In an embodiment, the composition of the invention comprises an RNA-guided DNA endonuclease and at least one pair of guide-RNAs, whereby (i) the endonuclease and the at least one pair of guide-RNAs are capable of assembling in vitro into at least one pair of ribonucleoproteins, and whereby (ii) each pair of ribonucleoproteins targets one locus in a cell; and, optionally, at least one donor-DNA construct wherein (i) the at least one donor-DNA construct has a 5′-end sequence which is at least partially complementary with the genome of the cell upstream of the target sequence of the first of the pair of ribonucleoproteins, and wherein (ii) the at least one donor-DNA construct has a 3′-end sequence which is at least partially complementary with the genome of the cell downstream of the target sequence of the second of the pair of ribonucleoproteins, and wherein (iii) the at least one donor-DNA construct serves as a template for homologous recombination repair of a first and a second break in the genome of the cell, wherein the first and second breaks are caused by the first and second of the pair of ribonucleoproteins, respectively; and a selectable marker.

The composition may comprise multiple pairs of ribonucleoproteins for introducing multiple genome edits, for example as described above in relation to the method of the invention. Thus, the composition may comprise
a. an RNA-guided DNA endonuclease and two pairs of guide-RNAs, a first and second pair of guide-RNAs
   i. whereby the endonuclease and each of the first and second pair of guide-RNAs are capable of assembling in vitro into two pairs of ribonucleoproteins, a first and second pair of ribonucleoproteins, and
   ii. whereby each pair of ribonucleoproteins targets one (i.e., a different) locus in a cell; and
b. two donor-DNA constructs, a first and second donor-DNA construct
   i. wherein the first donor-DNA construct has a 5′-end sequence which is at least partially complementary with the genome of the cell upstream of the target sequence of the first of the first pair of ribonucleoproteins, and
   ii. wherein the first donor-DNA construct has a 3′-end sequence which is at least partially complementary with the genome of the cell downstream of the target sequence of the second of the first pair of ribonucleoproteins, and
   iii. wherein the first donor-DNA construct serves as a template for homologous recombination repair of a first and a second break in the genome of the cell, wherein the first break is caused by the first of the first pair of ribonucleoproteins and the second break is caused by the second of the first pair of ribonucleoproteins; and
   iv. wherein the second donor-DNA construct has a 5′-end sequence which is at least partially complementary with the genome of the cell upstream of the target sequence of the first of the second pair of ribonucleoproteins, and
   v. wherein the second donor-DNA construct has a 3′-end sequence which is at least partially complementary with the genome of the cell downstream of the target sequence of the second of the second pair of ribonucleoproteins, and
   vi. wherein the second donor-DNA construct serves as a template for homologous recombination repair of a third and a fourth break in the genome of the cell, wherein the third break is caused by the first of the second pair of ribonucleoproteins and the fourth break is caused by the second of the second pair of ribonucleoproteins; and

a selectable marker.

The composition may comprise
a. an RNA-guided DNA endonuclease and three pairs of guide-RNAs, a first, second and third pair of guide-RNAs
   i. whereby the endonuclease and each of the first, second and third pair of guide-RNAs are capable of assembling in vitro into three pairs of ribonucleoproteins, a first, second and third pair of ribonucleoproteins, and
   ii. whereby each pair of ribonucleoproteins targets one (i.e., a different) locus in a cell; and
b. three donor-DNA constructs, a first, second and third donor-DNA construct
   i. wherein the first donor-DNA construct has a 5′-end sequence which is at least partially complementary with the genome of the cell upstream of the target sequence of the first of the first pair of ribonucleoproteins, and
   ii. wherein the first donor-DNA construct has a 3′-end sequence which is at least partially complementary with the genome of the cell downstream of the target sequence of the second of the first pair of ribonucleoproteins, and
   iii. wherein the first donor-DNA construct serves as a template for homologous recombination repair of a first and a second break in the genome of the cell, wherein the first break is caused by the first of the first pair of ribonucleoproteins and the second break is caused by the second of the first pair of ribonucleoproteins; and
   iv. wherein the second donor-DNA construct has a 5′-end sequence which is at least partially complementary with the genome of the cell upstream of the target sequence of the first of the second pair of ribonucleoproteins, and
   v. wherein the second donor-DNA construct has a 3′-end sequence which is at least partially complementary with the genome of the cell downstream of the target sequence of the second of the second pair of ribonucleoproteins, and
   vi. wherein the second donor-DNA construct serves as a template for homologous recombination repair of a third and a fourth break in the genome of the cell, wherein the third break is caused by the first of the second pair of ribonucleoproteins and the fourth break is caused by the second of the second pair of ribonucleoproteins; and
   vii. wherein the third donor-DNA construct has a 5′-end sequence which is at least partially complementary with the genome of the cell upstream of the target sequence of the first of the third pair of ribonucleoproteins, and
   viii. wherein the third donor-DNA construct has a 3′-end sequence which is at least partially complementary with the genome of the cell downstream of the target sequence of the second of the third pair of ribonucleoproteins, and
   ix. wherein the third donor-DNA construct serves as a template for homologous recombination repair of a fifth and a sixth break in the genome of the cell, wherein the fifth break is caused by the first of the third pair of ribonucleoproteins and the sixth break is caused by the second of the third pair of ribonucleoproteins; and

a selectable marker.

As the skilled person will appreciate, the above can be extrapolated to compositions comprising four or more donor-DNA constructs (and four or more corresponding pairs of guide RNAs) for introducing four or more genome edits in a cell, as described above in the context of the method of the invention.

### Cas Endonucleases

The term “Cas endonuclease” or “Cas proteins” means an RNA-guided DNA endonuclease associated with CRISPR that cleaves a target DNA sequence when coupled with a single guide RNA. The Cas endonuclease is guided by the single guide RNA(s) to recognize and cleave a specific target site in double stranded DNA in the genome of a cell. A commonly used type of CRIPSR-Cas system is the type II CRISPR-Cas system (comprised of subtypes including II-A, II-B, and II-C) from Streptococcus pyogenes, Streptococcus thermophilus, Staphylococcus aureus, Neisseria meningitidis or Campylobacter jejuni. The most common example is Cas9 endonuclease comprises two nuclease domains, an HNH (McrA-like) nuclease domain that cleaves the complementary DNA strand and a RuvC-like nuclease domain that cleaves the noncomplementary DNA strand. Target recognition and cleavage by the Cas9 endonuclease requires a chimeric single guide RNA consisting of a fusion of crRNA (a 20-nucleotide guide sequence and a partial direct repeat) and tracrRNA (transactivating crRNA) and a short conserved sequence motif downstream of the crRNA binding region, called a protospacer adjacent motif (PAM). In the CRISPR-Cas9 system derived from the bacterium Streptococcus pyogenes, the target DNA immediately precedes a 5′-NGG PAM. The RNA-guided Cas9 endonuclease activity creates site-specific double strand breaks, which are then repaired by either non-homologous end joining (NHEJ) or homologous recombination repair. It is understood that the term “Cas endonuclease” or “Cas proteins” encompasses variants thereof.

Alternatively, Type V CRISPR-Cas systems may be used (comprised of subtypes including V-A, V-B, V-E and V-F). A common example is Cas12a or Cpf1 (type V-A), C2c1 (type V-B), Cas12e (type V-E) or cas12f (type V-F) Isolated from for example Francisella novicida, Acidaminococcus sp., Lachnospiraceae sp., Planctomycetes sp., Acidibacillus sp, or Prevotella sp. A currently popular and widely available Type V CRISRP-Cas system is the “Cpf endonuclease” (also known as Cas12a endonuclease) means an RNA-guided DNA endonuclease associated with CRISPR that cleaves a target DNA sequence when coupled with a single guide RNA. The Cpf endonuclease is guided by the single guide RNA(s) to recognize and cleave a specific target site in double stranded DNA in the genome of a cell. The Cpf1-crRNA complex cleaves target DNA by identification of a protospacer adjacent motif (PAM) 5′-TTTN for the Acidaminococcus sp. Cpfl endonuclease and Lachnospiraceae sp. Cpfl endonuclease, and a PAM sequence 5′-TTN for the Francisella novicide Cpf1. After identification of the PAM, Cpfl introduces sticky-end DNA double-stranded break of 4-5 nucleotides overhang distal to the 3′ end of the targeted PAM which is then repaired by either non-homologous end joining (NHEJ) or homology-directed repair (HDR). It is understood that the term “Cpfl endonuclease” encompasses variants thereof. Mad endonucleases are modified Cas12a endonucleases: The term “Mad7 endonuclease” (also known as ErCas12a endonuclease) means an RNA-guided DNA endonuclease associated with CRISPR that cleaves a target DNA sequence when coupled with a single guide RNA. The Mad endonuclease is guided by the single guide RNA(s) to recognize and cleave a specific target site in double stranded DNA in the genome of a cell. CRISPR-Mad7 systems are closely related to the Type V (Cpf1-like) of Class-2 family of CAS enzymes. The MAD7-crRNA complex cleaves target DNA by identification of a protospacer adjacent motif (PAM) 5′-YTTN. After identification of the PAM, MAD7 introduces sticky-end DNA double-stranded break of 4-5 nucleotides overhang to the 3′ end of the targeted PAM which is then repaired by either non-homologous end joining (NHEJ) or homologous recombination repair. It is understood that the term “Mad7 endonuclease” encompasses variants thereof.

Cpfl endonuclease: The term “Cpf endonuclease” (also known as Cas12a endonuclease) means an RNA-guided DNA endonuclease associated with CRISPR that cleaves a target DNA sequence when coupled with a single guide RNA. The Cpf endonuclease is guided by the single guide RNA(s) to recognize and cleave a specific target site in double stranded DNA in the genome of a cell. The Cpfl-crRNA complex cleaves target DNA by identification of a protospacer adjacent motif (PAM) 5′-TTTN for the Acidaminococcus sp. Cpfl endonuclease and Lachnospiraceae sp. Cpfl endonuclease, and a PAM sequence 5′-TTN for the Francisella novicide Cpf1. After identification of the PAM, Cpfl introduces sticky-end DNA doublestranded break of 4-5 nucleotides overhang distal to the 3′ end of the targeted PAM which is then repaired by either non-homologous end joining (NHEJ) or homology-directed repair (HDR). It is understood that the term “Cpfl endonuclease” encompasses variants thereof.

Any polynucleotide-guided endonuclease may be used, both RNA and DNA-guided endonucleases are contemplated. The RNA-guided DNA endonuclease can be a Cas endonuclease, a Mad endonuclease or a Cpf endonuclease. The RNA-guided DNA endonuclease can be a Cas9 endonuclease, a Cas12a or Cpf1 endonuclease (type V-A), a C2c1 endonuclease (type V-B), a Cas12e endonuclease (type V-E) or a Cas12f (Type V-F) endonuclease or a Mad endonuclease.

In one aspect, the Cas endonuclease can be any Cas endonuclease or a functional fragment thereof useful in the methods of the present disclosure. In one embodiment, the Cas endonuclease is a Cas9 endonuclease. Examples of Cas9 endonucleases are the Cas9 endonucleases from the following bacterial species: Streptococcus sp. (e.g., S. pyogenes, S. mutans, and S. thermophilus), Campylobacter sp. (e.g., C. jejuni), Neisseria sp. (e.g., N. meningitidis), Francisella sp. (e.g., F. novicida), and Pasteurella sp. (e.g., P. 8 Jan. 13, 2022 multocida). For a discussion of Cas9 endonucleases, see Makarova et al., 2015, Nature 13: 722-736.

In another embodiment, the Cas9 endonuclease is a Streptococcus pyogenes Cas9 or homologue thereof. In another embodiment, the Cas9 endonuclease is a Streptococcus mutans Cas9 endonuclease. In another embodiment, the Cas9 endonuclease is a Streptococcus thermophilus Cas9 endonuclease. In another embodiment, the Cas9 endonuclease is a Campylobacter jejuni Cas9 endonuclease. In another embodiment, the Cas9 endonuclease is a Neisseria meningitidis Cas9 endonuclease. In another embodiment, the Cas9 endonuclease is a Francisella novicida Cas9 endonuclease. In another embodiment, the Cas9 endonuclease is a Pasteurella multocida Cas9 endonuclease.

In another aspect, the Mad endonuclease can be any Mad endonuclease or a functional fragment thereof useful in the methods of the present disclosure. In one embodiment, the Mad endonuclease is a MAD7 endonuclease. An example of a MAD7 endonuclease is the MAD7 endonuclease from Eubacterium rectale. For a discussion of the MAD7 endonuclease, see WO 2018/071672.

In another embodiment, the MAD7 endonuclease is a Eubacterium MAD7 endonuclease. In another embodiment, the Eubacterium MAD7 endonuclease is an Eubacterium rectale MAD7 endonuclease.

In one aspect, the Cpf endonuclease can be any Cpf endonuclease or a functional fragment thereof useful in the methods of the present disclosure. In one embodiment, the Mad endonuclease is a Cpf1 endonuclease. Examples of Cpfl endonucleases are the Cpfl endonucleases from Acidaminococcus sp., Lachnospiraceae sp., and Francisella novicide. For a discussion of the Cpfl endonuclease, see Zetsche et al., 2015, Cell 163(3) 759-771.

In another embodiment, the Cpfl endonuclease is an Acidaminococcus Cpfl endonuclease. In another embodiment, the Cpfl endonuclease is a Lachnospiraceae Cpfl endonuclease. In another embodiment, the Cpfl endonuclease is a Francisella Cpfl endonuclease. In another embodiment, the Cpf1 endonuclease is a Francisella novicide Cpfl endonuclease.

In another embodiment the endonuclease is a Cas12e or Cas12f endonuclease, see for example Wu Z, et al. Nat Chem Biol. 2021 November; 17(11):1132-1138. In yet another embodiment the endonuclease is a Cas12i or Cas12j see for example WO2021238556.

### Guide RNA

The term “guide RNA” in CRISPRCas9 genome editing refers to the re-programmable part that makes the system so versatile. In the natural S. pyogenes system the guide RNA is actually a complex of two RNA polynucleotides, a first crRNA containing about 20 nucleotides that determine the specificity of the Cas9 enzyme as well as the tracr RNA which hybridizes to the crRNA to form an RNA complex that interacts with Cas9. See Jinek M. et al., A programmable dual-RNA-guided DNA endonuclease in adaptive bacterial immunity. Science. 2012; 337: 816-21. The terms crRNA and tracrRNA are used interchangeably with the terms tracr-mate RNA and tracr RNA herein. Since the discovery of the CRISPR-Cas9 system single polynucleotide guide RNAs have been developed and successfully applied just as effectively as the natural two part guide RNA complex.

In the methods of the present disclosure, any guide RNA system can be used. In one embodiment, the guide RNA is the natural Streptomyces pyogenes system (Jinek et al., 2012, Science 337(6096): 816-821).

In another embodiment, the guide RNA, known as a single guide RNA (sgRNA), is an engineered single stranded chimeric RNA, which combines the scaffolding function of the bacterial transactivating CRISPR RNA (tracrRNA) with the specificity of the bacterial CRISPR RNA (crRNA). The last 17-20 bp at the 5′ end of the crRNA acts as a “guide”, which recruits the Cas9/gRNA complex to a specific DNA target site, directly upstream of a protospacer adjacent motif (PAM), through RNA-DNA base pairing.

In another embodiment, the single guide RNA comprises a first RNA comprising 17 to 20 or more nucleotides that are at least 85%, e.g., 90%, 95%, 96%, 97%, 98%, 99% or 100%, complementary to and capable of hybridizing to the target sequence.

In another embodiment, the first RNA comprising the 17 to 20 or more nucleotides are at least 90%, 95%, 97%, 98%, 99% or even 100% complementary to and capable of hybridizing to the target sequence.

In another embodiment, the single guide RNA is a Streptomyces pyogenes Cas9 guide RNA. In another embodiment, the guide RNA is an Eubacterium rectale MAD7 guide RNA. In another embodiment, the guide RNA is a Cpfl guide RNA.

In another embodiment the guide RNA is a cas12e guide RNA. In another embodiment the guide RNA is a cas12f guide RNA. In yet another embodiment the guide RNA is a Cas12i or Cas12j guide RNA.

### Donor-DNA Construct

The term “donor-DNA construct” or “donor-DNA” means a polynucleotide that comprises a nucleotide sequence for modifying a target site in the genome of a fungal cell. The donor DNA can be double-stranded DNA or single-stranded DNA. The nucleotide sequence of the donor DNA can comprise any nucleotide sequence of interest such as a gene or a region of a gene, one or more nucleotides for introducing a mutation into a gene, a gene disruption sequence, etc. The donor DNA comprises a first region of homology (a 5′-end sequence) and a second region of homology (a 3′-end sequence) to corresponding regions of the target locus for homologous recombination repair of the first and second break introduced by the pair of ribonucleoproteins (The 5′-end sequence and 3′-end sequences are sometimes also referred to as flanking regions or homology regions or flanking homology regions). In other words, the donor-DNA construct has a 5′-end sequence and a 3′-end sequence that are each complementary, or at least partially complementary, to the regions upstream and downstream of the breaks caused by the RNA-guided DNA endonucleases, respectively. The donor-DNA construct may comprise an additional sequence 5′ of the 5′-end sequence and/or additional sequence 3′ of the 3′-end sequence. In other words, the 5′-end and 3′-end sequences of the donor-DNA do not need to be at the 5′ and 3′ termini, respectively. Nucleotide sequences are said to be complementary when they can interact by nucleotide base-pairing to form a double stranded DNA or RNA construct. In the context of homologous recombination repair, the term complementary would thus refer to the ability of the donor DNA to form a stable base-pairing with the single stranded DNA that is formed during the process of homologous recombination repair and where it can thus serve as a template to repair a double strand break. Hence, the term “donor DNA” or donor-DNA construct is also understood herein to mean “DNA repair template”.

Formation and transformation of Ribonucleoproteins or RNPs

The term “ribonucleoprotein(s)”, “RNP(s)” or “polynucleotide-guided endonuclease(s)” in the context of the current invention refers to the complex that is formed between an RNA-guided DNA endonuclease (e.g., Cas endonuclease or Cas protein) and a guide RNA. A single guide RNA and an RNA-guided DNA endonuclease need to interact and form a ribonucleoprotein in order to bind to the target DNA sequence and activate the endonuclease activity of the endonuclease. RNPs can be formed intracellular when both the endonuclease and guide RNA are present in the cell. This can be achieved for example by providing the coding region of the endonuclease and the coding region for the guide RNA on a plasmid/expression vector under control of a promoter whereby when the plasmid vector is introduced in the cell both guide RNA and endonuclease are being produced for example when an inducer of the promoter system is provided.

For the purpose of this invention, the RNPs can also be pre-assembled in vitro i.e. outside of the cell and thereafter introduced into the cell by means of a suitable transformation technique such as for example lipofection, electroporation, microinjection, PEG-mediated transformation. For this RNPs may be assemble in a microfuge tube by combining and mixing a purified RNA-guided DNA endonuclease with a synthetically produced guide RNA. The endonuclease and guide RNA may be mixed in equimolar concentrations, alternatively an excess of guide RNA may be used, such as a 3:1 guide RNA:endonuclease ratio. The mixture of guide RNA and endonuclease may be incubated in a suitable buffer such as a Tris buffer at pH 7.5 and incubated at a suitable temperature such as room temperature during for example 5 to 30 minutes prior to transformation.

The examples of the current disclosure provide a detailed example of how the formation and transformation of RNPs can be achieved in filamentous fungal cells.

### Target Sequence

A target sequence in the context of the invention is understood as the site at which an RNP will introduce a break or nick in the genome of the cell. i.e. a guide RNA is designed such that a break will occur in the target sequence on the genome of the cell. In the method of the present invention, two guide RNAs are used (i.e., a first and second), one in each of the pair of ribonucleoproteins. The first guide RNA has a target sequence that is at the 5′-end of the target locus in the genome, to guide the endonuclease in the first ribonucleoprotein to create the first break by endonuclease cleavage. The second guide RNA has a target sequence at the 3′-end of the target locus in the genome, to guide the endonuclease in the second ribonucleoprotein to create the second break by endonuclease cleavage (see FIG. 1). The target sequence may be located in the vicinity of a protospacer adjacent motif (PAM) sequence for the polynucleotide-guided endonuclease or RNP; preferably the at least one target sequence to be modified is located from 10 to 1,000 nucleotides away from the protospacer adjacent motif (PAM) sequence for the polynucleotide-guided endonuclease in the genome of the cell; preferably the at least one genome target sequence to be modified is located from 10 to 500 nucleotides away from the protospacer adjacent motif (PAM) sequence for the polynucleotide-guided endonuclease in the genome of the cell; more preferably the at least one genome target sequence to be modified is located from 10 to 250 nucleotides away from the protospacer adjacent motif (PAM) sequence for the polynucleotide-guided endonuclease in the genome of the cell; even more preferably the at least one genome target sequence to be modified is located from 10 to 100 nucleotides away from the protospacer adjacent motif (PAM) sequence for the polynucleotide-guided endonuclease in the genome of the cell.

The actual cut, nick, or double-stranded break (also referred to as “break”) in the genome target sequence may be made within a “protospacer-complementary” sequence located immediately next to the PAM sequence in the genome. The protospacer-complementary sequence is usually 20 nucleotides in length or so, in order to allow its hybridization to the corresponding protospacer sequence of the guide RNA, but even shorter sequences have been shown to work, such as, a nucleotide protospacer in the guide and corresponding protospacer-complementary nucleotide sequence in the genome. The at least one genome target locus to be modified may be located anywhere in the genome but will often be within a coding sequence or open reading frame. In the present invention, the target locus is targeted by a pair of RNPs where for example the first RNP may cause a break at a target sequence at the 5′-end of the target locus and the second RNP may cause a break at a target sequence at the 3′-end of the target locus.

Each protospacer-complementary sequence in the genome may need to have a suitable protospacer adjacent motif (PAM) located next to it to allow the corresponding polynucleotide-guided endonuclease to bind and cut or nick the genome. The term “protospacer adjacent motif’ or “PAM” means a 2-6 base pair DNA sequence immediately downstream or upstream of the target site in the genome, which is recognized directly by an RNA-guided DNA endonuclease, e.g., Cas9, Cas12a, Cas12e, Cas12f, Cas12i and Cas12j endonucleases (e.g. a Cas9, Mad7, or Cas12a endonuclease), to promote cleavage of the target site by the RNA-guided DNA endonuclease. The Cas9 endonuclease from Streptococcus pyogenes recognizes 5′-NGG on the 3′ end of the gRNA sequence. The Mad7 endonuclease from Eubacterium rectale recognizes 5′-TTTV on the 5′ end of the gRNA sequence, but 5′-YTTV and YTTN also work to some extent. The cas12a endonuclease from Acidaminococcus sp. And Lachnospiraceae sp. recognize 5′-TTTN and the cas12a endonuclease from Francisella novicide recognizes 5′-TTN-3′ on the 5′ end of the gRNA. For an overview of other PAM sequences, see, for example, Shah, S. A. et al, Protospacer recognition motifs, RNA Biol. 2013 May 1; 10(5): 891-899.

### Selectable Marker

A “selectable marker” or “selection marker” or “selection cassette” is a gene introduced into a cell that confers a trait suitable for artificial selection i.e. the cell receiving the selectable marker is capable of growing on or in a growth media containing or lacking a substance preventing cells without the selectable marker from growing or killing the cells lacking the selectable marker. Selectable markers are often antibiotic resistance genes. Examples include the bleoR gene encoding the phleomycin resistance protein conferring resistance against the antibiotic phleomycin, the hygB gene encoding the Hygromycin B resistance protein conferring resistance against the antibiotic Hygromycin B, nptII or neo gene encoding the neomycin phosphotransferase conferring resistance against the antibiotic Neomycin.

A selectable marked often comes with a constitutive promoter so that the corresponding gene is expressed. Additionally, a marker can be equipped with a terminator to prevent readthrough of said promoter. For example, a commonly used selectable marker cassette is constructed of hygB encoding hygromycin phosphotransferase gene, as well as the the oliC promoter and the trpC terminator of Aspergillus nidulans.

Selectable marker can be an antibiotic but also an auxotrophic marker where the presence of the selectable marker allows the cell to grow in the absence of an essential nutrient. For example, the PyrG gene encoding the orotidine-5′-decarboxylase which allows an auxotrophic strain to grow in the absence of uridine. A benefit over an auxotrophic marker is that an antibiotic resistance marker does not require the construction of laborious auxotrophic strains.

### Screening the Cell for the Genome Edits

With the term “screening” or “screening the cell for the genome edits” is meant molecular biological techniques that allow the quick and straightforward identification of the presence or absence of a locus or target site. As an example, this can often be done by using a standard PCR using primers specifically designed to amplify the target locus. Any insertion or deletion will be detected by comparing the relative size of the resulting PCR products. Alternatively, such a PCR product may be sequenced to identify smaller alterations or confirming the correct deletion or integration. Alternatively, the PCR primers can be designed such that one primer is complementary to a region inserted into the genome (such as an expression cassette) or complementary to a region spanning the repaired double-strand breaks and the corresponding primer complementary to a region outside of the locus that was edited, and hence will only yield a PCR fragment of correct size when the genome edit is present.

### Methods of Producing Compounds of Interest

The invention provides methods for the production of a compound (e.g., a protein) of interest. The compound of interest may be a compound as described herein, for example an antibody or a functional fragment thereof, a carbohydrate-binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor, a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a VHH. The methods comprise providing a cell obtained using methods of the invention, which is characterized by having been modified and where this modification leads to the introduction/insertion of a protein of interest in one or more genomic targets using the method of the invention. Additionally, the cell of the invention may be further modified so to affect the production, stability and/or function of at least one polypeptide; and having for example a reduction or deficiency in protease activity of one or more proteases. The method further comprises culturing said modified microbial host cell under conditions conducive to the expression of the compound of interest. The method may further optionally comprise a step of isolating the compound of interest from the culture medium or fermentation broth.

In some embodiments, the methods may comprise a step of inducing expression of the compound of interest by the microbial host cell. For example, if the compound of interest is encoded by a nucleotide sequence that is operably linked to an inducible promoter, the method may comprise a step of inducing the expression of the compound of interest. A common inducible promoter that may be used is the inducible cbh1 or cbh2 promoter, in which administration of lactose will initiate expression. Other inducible promoters could of course be used. If the sequence encoding the compound of interest is under the control of a constitutive promoter, no specific step of induction of expression may be required.

Fermentation or culture of the microbial host cells may occur in a solid fermentation or culture setting or a liquid fermentation or culture setting. Solid-state fermentation or culture may comprise seeding the microbial host cell on a solid culture substrate, and methods of solid-state fermentation or culture are known the skilled person. Liquid fermentation or culture may comprise culturing the microbial host cell in a liquid cell culture medium.

The method may also comprise a step of isolating the compound of interest produced by the microbial host cell, for example isolating the compound of interest from the fermentation broth or cell culture medium.

The method may further comprise a step of formulating the compound of interest into a agrochemical or pharmaceutical composition. The step of formulating the compound of interest into an agrochemical composition may comprise formulating the compound of interest with one or more agrochemically acceptable excipients. The step of formulating the compound of interest into a pharmaceutical composition may comprise formulating the compound of interest with one or more pharmaceutically acceptable excipients.

The present invention therefore provides compounds of interest obtained by a method of the present invention. The present invention also therefore provides an agrochemical or pharmaceutical composition obtained by a method of the present invention.

Any methods comprising or requiring the culturing or fermentation of the modified microbial host cell comprise the culture or fermentation of the host cell is a suitable medium. Generally, the medium will comprise any and all nutrients required for the microbial host cell to grow. The skilled person will be aware of the required components of the cell culture media or fermentation broth, which may differ depending on the species of microbial host cell being cultured. In some embodiments, the cell culture media or fermentation broth may comprise a nitrogen source, such as ammonium or peptone.

### Further Definitions

As used herein, the terms “nucleic acid molecule”, “polynucleotide”, “polynucleic acid”, “nucleic acid”, “nucleotide sequence” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, promotor regions, isolated RNA of any sequence, nucleic acid probes, and primers. The nucleic acid molecule may be linear or circular.

The term “recombinant polynucleotide” refers to a nucleic acid molecule that was introduced in the filamentous fungus cell by means of recombinant DNA technology as is well known in the art and described in for example Molecular Cloning: A Laboratory Manual, 3rd ed., Vols 1, 2 and 3 J. F. Sambrook and D. W. Russell, ed., Cold Spring Harbor Laboratory Press, 2001, 2100 pp. Recombinant DNA molecules can have its origin in a species other than the filamentous fungal cell or can be a polynucleotide native to the filamentous fungal cell.

With a “compound of interest” it is meant any recombinant protein such as an antibody or a functional fragment thereof, a carbohydrate-binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a VHH.

In some embodiments, the compound of interest is a therapeutic protein, biosimilar, multi-domain protein, peptide hormone, antimicrobial peptide, peptide, carbohydrate-binding module, enzyme, cellulase, protease, protease inhibitor, aminopeptidase, amylase, carbohydrase, carboxypeptidase, catalase, chitinase, cutinase, deoxyribonuclease, esterase, alpha-galactosidase, beta-galactosidase, glucoamylase, alpha-glucosidase, beta-glucosidase, invertase, laccase, lipase, mannanase, mutanase, oxidase, pectinolytic enzyme, peroxidase, phospholipase, phytase, phosphatase, polyphenoloxidase, redox enzyme, proteolytic enzyme, ribonuclease, transglutaminase or xylanase.

In some embodiments, the compound of interest is a VHH. In more specific embodiments, the VHH may be a VHH bind a specific lipid fraction of the cell membrane of a fungal spore. Such VHHs may exhibit fungicidal activity through retardation of growth and/or lysis and explosion of spores, thus preventing mycelium formation. The VHH may therefore have fungicidal or fungistatic activity.

In some embodiments, the VHH may be a VHH that is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:
fractionating hyphae of a fungus (for example Botrytis cinerea or other fungus) by total lipid extract thin-layer chromatography and selecting the fraction with a Retention Factor (RD higher than the ceramide fraction and lower than the non-polar phospholipids fraction.

The invention also provides a polypeptide, wherein said at least one polypeptide is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:
fractionating hyphae of a fungus (for example Botrytis cinerea or other fungus) by total lipid extract thin-layer chromatography and selecting the fraction with a Retention Factor (RD higher than the ceramide fraction and lower than the non-polar phospholipids fraction.

The VHHs are generally capable of binding to a fungus. The VHH thereby causes retardation of growth of a spore of the said fungus and/or lysis of a spore of the said fungus. That is to say, binding of the VHH to a fungus results in retardation of growth of a spore of the said fungus and/or lysis of a spore of the said fungus.

The VHHs may (specifically) bind to a membrane of a fungus or a component of a membrane of a fungus. In some embodiments, the VHHs do not (specifically) bind to a cell wall or a component of a cell wall of a fungus. For example, in some embodiments, the VHHs do not (specifically) bind to a glucosylceramide of a fungus.

The VHHs may be capable of (specifically) binding to a lipid-containing fraction of the plasma membrane of a fungus, such as for example a lipid-containing fraction of Botrytis cinerea or other fungus. Said lipid-containing fraction (of Botrytis cinerea or otherwise) may be obtainable by chromatography. The chromatography may be performed on a crude lipid extract (also referred to herein as a total lipid extract, or TLE) obtained from fungal hyphae and/or conidia. The chromatography may be, for example, thin-layer chromatography or normal-phase flash chromatography. The chromatography (for example thin-layer chromatography) may be performed on a substrate, for example a glass plate coated with silica gel. The chromatography may be performed using a chloroform/methanol mixture (for example 85/15% v/v) as the eluent.

For example, said lipid-containing fraction may be obtainable by a method comprising:
fractionating hyphae and/or conidia of a fungus (for example Botrytis cinerea or other fungus) by total lipid extract thin-layer chromatography and selecting the fraction with a Retention Factor (RD higher than the ceramide fraction and lower than the non-polar phospholipids fraction.

In a more specific embodiment, the lipid-containing fraction may be obtainable by a method comprising:
fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (RD higher than the ceramide fraction and lower than the non-polar phospholipids fraction.

Alternatively, the fraction may be obtained using normal-phase flash chromatography. In such a method, the method may comprise:
fractionating hyphae and/or conidia of a fungus (for example Botrytis cinerea or other fungus) by total lipid extract normal-phase flash chromatography, and selecting the fraction with a Retention Factor (RD higher than the ceramide fraction and lower than the non-polar phospholipids fraction.

In a more specific embodiment, the lipid-containing fraction may be obtainable by a method comprising:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH and using CH₂Cl₂/MeOH (for example 85/15%, v/v) as the eluent, followed by filtration of the fractions through a filter.

In a more specific embodiment, the lipid-containing fraction may be obtainable by a method comprising:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH loading the TLE on to a phase flash cartridge (for example a flash cartridge with 15 μm particles), running the column with CH₂Cl₂/MeOH (85/15%, v/v) as the eluent, and filtering the fractions through a filter (for example a 0.45 μm syringe filter with a nylon membrane) and drying the fractions.

The fractions from the chromatography may be processed prior to testing of binding of the VHH to the fraction or of interaction with the fraction. For example, liposomes comprising the fractions may be prepared. Such a method may comprise the use of thin-film hydration. For example, in such a method, liposomes may be prepared using thin-film hydration with the addition of 1,6-diphenyl-1,3,5-hexatriene (DPH). Binding and/or disruption of the membranes by binding of the VHH may be measured by a change in fluorescence before and after polypeptide binding (or by reference to a suitable control).

Accordingly, in some embodiments, the VHHs may (specifically) bind to a lipid-containing chromatographic fraction of the plasma membrane of a fungus, optionally wherein the lipid-containing chromatographic fraction is prepared into liposomes prior to testing the binding of the polypeptide thereto.

Binding of the VHH to a lipid-containing fraction of a fungus may be confirmed by any suitable method, for example bio-layer interferometry. Specific interactions with the lipid-containing fractions may be tested. For example, it may be determined if the polypeptide is able to disrupt the lipid fraction when the fraction is prepared into liposomes, for example using thin-film hydration.

In methods involving chromatography, an extraction step may be performed prior to the step of chromatography. For example, fungal hyphae and/or conidia may be subjected to an extraction step to provide a crude lipid extract or total lipid extract on which the chromatography is performed. For example, in some embodiments, fungal hyphae and/or conidia (for example fungal hyphae and/or conidia of Fusarium oxysporum or Botrytis cinerea) may be extracted at room temperature, for example using chloroform:methanol at 2:1 and 1:2 (v/v) ratios. Extracts so prepared may be combined and dried to provide a crude lipid extract or TLE.

Accordingly, in some embodiments, the VHH may be capable of (specifically) binding to a lipid-containing fraction of the plasma membrane of a fungus (such as Fusarium oxysporum or Botrytis cinerea), wherein the lipid-containing fraction of the plasma membrane of the fungus is obtained or obtainable by chromatography. The chromatography may be normal-phase flash chromatography or thin-layer chromatography. Binding of the VHH to the lipid to the lipid-containing fraction may be determined according to bio-layer interferometry. In some embodiments, the chromatography step may be performed on a crude lipid fraction obtained or obtainable by a method comprising extracting lipids from fungal hyphae and/or conidia from a fungal sample. The extraction step may use chloroform:methanol at 2:1 and 1:2 (v/v) ratios to provide two extracts, and then combining the extracts.

In methods relating to thin-layer chromatography, the chromatography may comprise the steps of:
fractionating hyphae of the fungus by total lipid extract thin-layer chromatography and selecting the fraction with a Retention Factor (RD higher than the ceramide fraction and lower than the non-polar phospholipids fraction.

In some methods relating to thin-layer chromatography, the chromatography may comprise the steps of:
fractionating hyphae and/or conidia of a fungus (for example Botrytis cinerea or other fungi) by total lipid extract thin-layer chromatography on a silica-coated glass slide using a chloroform/methanol mixture (for example 85/15% v/v) as the eluent and selecting the fraction with a Retention Factor (RD higher than the ceramide fraction and lower than the non-polar phospholipids fraction.

In methods relating to normal-phase flash chromatography, the chromatography may comprise the steps of:
fractionating hyphae and/or conidia of a fungus (for example Botrytis cinerea or other fungus) by total lipid extract normal-phase flash chromatography, and selecting the fraction with a Retention Factor (RD higher than the ceramide fraction and lower than the non-polar phospholipids fraction.

In some methods relating to normal-phase flash chromatography, the chromatography may comprise the steps of:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH and using CH₂Cl₂/MeOH (for example 85/15%, v/v) as the eluent, followed by filtration of the fractions through a filter.

In some methods relating to normal-phase flash chromatography, the chromatography may comprise the steps of:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH loading the TLE on to a phase flash cartridge (for example a flash cartridge with 15 μm particles), running the column with CH₂Cl₂/MeOH (85/15%, v/v) as the eluent, and filtering the fractions through a filter (for example a 0.45 μm syringe filter with a nylon membrane) and drying the fractions.

In some embodiments, the compound of interest is VHH-1, VHH-2 or VHH-3. For example, in some embodiments, the compound of interest is a VHH comprising or consisting of a sequence selected from the group consisting of SEQ ID NOs: 1, 2, 6, 10, 14 and 15.

In some embodiments, the compound of interest is a VHH comprising:
(a) a CDR1 comprising or consisting of a sequence selected from the group consisting of SEQ ID NOs 3, 7 and 11;
(b) a CDR2 comprising or consisting of a sequence selected from the group consisting of SEQ ID NOs: 4, 8 and 12; and
(c) a CDR3 comprising or consisting of a sequence selected from the group consisting of SEQ ID NOs: 5, 9 and 13.

In some embodiments, the compound of interest is a VHH comprising:
(a) a CDR1 comprising or consisting of the sequence of SEQ ID NO: 3, a CDR2 comprising or consisting of the sequence of SEQ ID NO: 4 and a CDR3 comprising or consisting of the sequence of SEQ ID NO: 5;
(b) a CDR1 comprising or consisting of the sequence of SEQ ID NO: 7, a CDR2 comprising or consisting of the sequence of SEQ ID NO: 8 and a CDR3 comprising or consisting of the sequence of SEQ ID NO: 9 or
(c) a CDR1 comprising or consisting of the sequence of SEQ ID NO: 11, a CDR2 comprising or consisting of the sequence of SEQ ID NO: 12 and a CDR3 comprising or consisting of the sequence of SEQ ID NO: 13.

In some embodiments, the compound of interest is a VHH comprising a CDR1 comprising or consisting of the sequence of SEQ ID NO: 3, a CDR2 comprising or consisting of the sequence of SEQ ID NO: 4 and a CDR3 comprising or consisting of the sequence of SEQ ID NO: 5.

In some embodiments, the compound of interest is a VHH comprising SEQ ID NO: 1.

In some embodiments, the compound of interest is a VHH comprising SEQ ID NO: 2.

In some embodiments, the compound is a VHH disclosed in WO2014/177595 or WO2014/191146, the entire contents of which are incorporated herein by reference. More specifically the compound is a VHH comprising an amino acid sequence chosen from the group consisting of SEQ ID NO's: 1 to 84 from WO2014/177595 or WO2014/191146.

Thus, the microbial host cells of the invention can be used to produce compounds of interest, in particular VHHs, such as the VHHs disclosed herein, as well as other VHHs, such as those disclosed in WO2014/177595 or WO2014/191146. In some embodiments, the VHHs are fused to a carrier peptide.

With “capable of expressing a compound of interest” it is meant that the microbial host cell is modified in such a way that it contains the genetic information of a compound of interest that is under control of a promoter sequence that drives the expression of said compound either in a continuous manner or during conditions suitable for expression. For example, in some embodiments, the microbial host cell may comprise a polynucleotide coding for the compound of interest. The polynucleotide may be in the form of a plasmid or a vector. The polynucleotide may be introduced into the microbial host cell according to any suitable method known to the skilled person. For example, the polynucleotide may be introduced into the cell by transformation, for example protoplast-mediated transformation (PMT), Agrobacterium-mediated transformation (AMT), electroporation, biolistic transformation (particle bombardment), or shock-wave-mediated transformation (SWMT). The compound of interest is therefore a recombinant or heterologous compound of interest, since it is not encoded by the wild-type genome of the microbial host cell.

The compound of interest may be under the control of (i.e. may be operably linked to) a promoter sequence. The promoter sequence may promote the expression of the compound of interest in and by the modified microbial host cell. In some embodiments, the compound of interest may be operably linked to a constitutive promoter, or the compound of interest may be operably linked to an inducible promoter. When linked to a inducible promoter, methods of the invention may comprise a step of inducing expression of the compound of interest by the microbial host cell.

With a “promoter sequence” it is meant a nucleotide sequence that is preferably recognized by a polypeptide, for example a regulator of transcription or at the very least allows the correct formation of a RNA-polymerase complex in such a way that expression of a compound of interest, of which the polynucleotide is located downstream of the promoter sequence as is well known in the art, is established in a continuous manner or during conditions suitable for expression, as to produce the compound of interest or a compound involved in the production of the compound of interest. The promoters are generally promoters that are functional in fungi. These promoters can be but are not limited to alcA Alcohol dehydrogenase I, amyB TAKA-amylase A, bli-3 Blue light-inducible gene, bphA Benzoate p-hydrolase, catR Catalase, cbh1 (cbhI) Cellobiohydrolase I, cbh2 (cbhII) cellobiohydrolase 2, cel5a endoglucanase 2, cel12a endogluconase 3, cre1 Glucose repressor, exyIA endoxylanase, gas 1,3-beta-glucanosyltransferase, glaA Glucoamylase A, gla1 Glucoamylase, mir1 Siderophore transporter, niiA Nitrite reductase, qa-2 Catabolic 3-dehydroquinase, Smxyl endoxylanase, tcu-1 Copper transporter, thiA thiamine thiazole synthase, vvd Blue light receptor, xyl1 endoxylanase, xylP endoxylanase, xyn1 endoxylanase 1, xyn2 endoxylanase 2, xyn3 endoxylanase 3, zeaR regulator of transcription, cDNA1, eno1 enolase, gpd1 glyceraldehyde-3-phosphate dehydrogenase, pdc1 pyruvate decarboxylase, pki1 pyruvate kinase, tef1 transcription elongation factor 1α, rp2 ribosomal protein, stp1 sugar transporter or tauD3 tauD like dioxygenase.

As used herein, the terms “polypeptide”, “protein”, “peptide”, and “amino acid sequence” are used interchangeably, and refer to a polymeric form of amino acids of any length, which can include coded and non-coded amino acids, chemically or biochemically modified or derivatized amino acids, and polypeptides having modified peptide backbones.

In some embodiments the compound of interest is a polypeptide that is fused to a second polypeptide and where the second polypeptide is a “carrier peptide”. In other words, the microbial host cell may comprise a polynucleotide sequence encoding a polypeptide fused to a carrier peptide. Carrier peptides are peptides that may be produced and secreted by the microbial host cell. Carrier peptides may be abundant or produced in quantities that exceed other peptides not suitable to be used as a carrier peptide. Carrier peptides may be native to the microbial host cell. Thus, carrier peptides may serve to increase the production and/or the secretion of the compound of interest as compared to the production and/or secretion of a compound of interest not fused to a carrier peptide. Carrier peptides may be, but are not limited to, a glucoamylase Gla peptide, a cellobiohydrolase Cbh1 peptide or a cellobiohydrolase cbh2 peptide. Carrier peptides may consist of a functional fragment of, but not limited to, glucoamylase GlaA peptide, alpha-amylase peptide, a cellobiohydrolase Cbh1 peptide or a cellobiohydrolase cbh2 peptide. A functional fragment of a carrier peptide may be limited to the N-terminal region of, but not limited to, glucoamylase GlaA peptide, a cellobiohydrolase Cbh1 peptide or a cellobiohydrolase cbh2 peptide. Alternatively the functional fragment of a carrier peptide may be limited to the catalytic domain of the carrier peptide, such as the catalytic domain of the cbh1 carrier peptide. The N-terminal region may consist of only the signal peptide or signal sequence of, but not limited to glucoamylase Gla peptide, a cellobiohydrolase Cbh1 peptide or a cellobiohydrolase cbh2 peptide. The signal peptide or signal sequence may allow for the secretion of the compound of interest. In more preferred embodiments the carrier peptide is fused to the N-terminus of the compound of interest. In some embodiments the compound of interest and the carrier peptide may be separated by a proteolytic cleavage site. That is to say, a third peptide containing a proteolytic cleavage site can be present between the compound of interest and the carrier peptide. In a more preferred embodiment the proteolytic cleavage site is fused to the C-terminus of the carrier-peptide and the N-terminus of the compound of interest. Thus, the polypeptide may be a fusion protein comprising, in a 5′ to 3′ order, a carrier peptide, a proteolytic cleavage site, and the compound of interest. The proteolytic cleavage site may be, but is not limited to, the KexB proteolytic cleavage site. The presence of a proteolytic cleavage site allows for the compound of interest to be separated from the carrier peptide by action of a protease. This protease may be but is not limited to the KexB protease. In some embodiments this separation takes place at the time of secretion or immediately after secretion of the fusion protein. In other embodiments the protease separating the compound of interest can be added to the fermentation medium. In some embodiments the protease separating the compound of interest can be added during or after purification of the fusion protein. In a preferred embodiment the separation of the compound of interest from the carrier peptide can occur by protease activity native to the microbial host cell.

As used herein, the term “homology” denotes at least secondary structural similarity between two macromolecules, particularly between two polypeptides or polynucleotides, from same or different taxons, wherein said similarity is due to shared ancestry. Hence, the term “homologous” denotes so-related macromolecules having said secondary and optionally tertiary structural similarity.

For comparing two or more nucleotide sequences, sequence “identity” may be used, in which the “(percentage of) sequence identity” between a first nucleotide sequence and a second nucleotide sequence may be calculated using methods known by the person skilled in the art.

The terms “sequence identity”, “% identity” are used interchangeable herein. For the purposes of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared.

Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package may be used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/).

For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids over their entire length, they are identical or have 100% identity. Amino acid sequences and nucleic acid sequences are said to be “exactly the same” or “identical” if they have 100% sequence identity over their entire length.

In determining the degree of sequence identity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art.

In light of the current invention, the above concepts on sequence identity and methods for determining sequence identity can be applied to the concept of complementarity or the concept of a nucleotide sequences being complementary to a second nucleotide sequence. That is, for full complementarity the sequence identity of the first nucleotide sequence (e.g. the guide RNA or the donor-DNA construct) needs to be 100% identical to the complement of the second nucleotide sequence (e.g. the target sequence). Partially complementary, may be used to refer to two sequences that are at least 70%, at least 80%, at least 90%, or at least 95% complementary, i.e., one sequence has the recited sequence identity against the complement of the other sequence.

As used herein, the term “antibody” refers to polyclonal antibodies, monoclonal antibodies, humanized antibodies, chimeric antibodies, minibodies, diabodies, nanobodies, nanoantibodies, affibodies, alphabodies, designed ankyrin-repeat domains, anticalins, knottins, engineered CH2 domains, single-chain antibodies, or fragments thereof such as Fab F(ab)₂, F(ab)₃, scFv, a single domain antibody, a heavy chain variable domain of an antibody, a heavy chain variable domain of a heavy chain antibody (VHH), the variable domain of a camelid heavy chain antibody, a variable domain of the a new antigen receptor (vNAR), a variable domain of a shark new antigen receptor, or other fragments or antibody formats that retain the antigen-binding function of a parent antibody. As such, an antibody may refer to an immunoglobulin, or fragment or portion thereof, or to a construct comprising an antigen-binding portion comprised within a modified immunoglobulin-like framework, or to an antigen-binding portion comprised within a construct comprising a nonimmunoglobulin-like framework or scaffold.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab)2, Fv, and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies. As used herein, the term “polyclonal antibody” refers to an antibody composition having a heterogeneous antibody population. Polyclonal antibodies are often derived from the pooled serum from immunized animals or from selected humans.

“Heavy chain variable domain of an antibody or a functional fragment thereof” (also indicated hereafter as VHH), as used herein, means (i) the variable domain of the heavy chain of a heavy chain antibody, which is naturally devoid of light chains, including but not limited to the variable domain of the heavy chain of heavy chain antibodies of camelids or sharks or (ii) the variable domain of the heavy chain of a conventional four-chain antibody (also indicated hereafter as VH), including but not limited to a camelized (as further defined herein) variable domain of the heavy chain of a conventional four-chain antibody (also indicated hereafter as camelized VH).

As used herein, the terms “complementarity determining region” or “CDR” within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains. As further described hereinbelow, the amino acid sequence and structure of a heavy chain variable domain of an antibody can be considered, without however being limited thereto, to be comprised of four framework regions or “FR's”, which are referred to in the art and hereinbelow as “framework region 1” or “FR1”; as 40 “framework region 2” or “FR2”; as “framework region 3” or “FR3”; and as “framework region 4” or “FR4”, respectively, which framework regions are interrupted by three complementary determining regions or “CDR's”, which are referred to in the art as “complementarity determining region 1” or “CDR1”; as “complementarity determining region 2” or “CDR2”; and as “complementarity determining region 3” or “CDR3”, respectively.

As also further described hereinbelow, the total number of amino acid residues in a heavy chain variable domain of an antibody (including a V_{HH} or a V_{H}) can be in the region of 110-130, is preferably 112-115, and is most preferably 113. It should however be noted that parts, fragments or analogs of a heavy chain variable domain of an antibody are not particularly limited as to their length and/or size, as long as such parts, fragments or analogs retain (at least part of) the functional activity, such as the pesticidal, biocidal, biostatic activity, fungicidal or fungistatic activity (as defined herein) and/or retain (at least part of) the binding specificity of the original a heavy chain variable domain of an antibody from which these parts, fragments or analogs are derived from. Parts, fragments or analogs retaining (at least part of) the functional activity, such as the pesticidal, biocidal, biostatic activity, fungicidal or fungistatic activity (as defined herein) and/or retaining (at least part of) the binding specificity of the original heavy chain variable domain of an antibody from which these parts, fragments or analogs are derived from are also further referred to herein as “functional fragments” of a heavy chain variable domain.

A method for numbering the amino acid residues of heavy chain variable domains is the method described by Chothia et al. (Nature 342, 877-883 (1989)), the so-called “AbM definition” and the so-called “contact definition”. Herein, this is the numbering system adopted.

Alternatively, the amino acid residues of a variable domain of a heavy chain variable domain of an antibody (including a V_{HH} or a V_{H}) may be numbered according to the general numbering for heavy chain variable domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to above (see for example FIG. 2 of said reference).

For a general description of heavy chain antibodies and the variable domains thereof, reference is inter alia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx NV; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by Ablynx; Hamers-Casterman et al., Nature 1993 Jun. 3; 363 (6428): 446-8.

Generally, it should be noted that the term “heavy chain variable domain” as used herein in its broadest sense is not limited to a specific biological source or to a specific method of preparation. For example, as will be discussed in more detail below, the heavy chain variable domains of the invention can be obtained (1) by isolating the V_{HH} domain of a naturally occurring heavy chain antibody; (2) by isolating the V_{H} domain of a naturally occurring four-chain antibody (3) by expression of a nucleotide sequence encoding a naturally occurring V_{HH} domain; (4) by expression of a nucleotide sequence encoding a naturally occurring V_{H} domain (5) by “camelization” (as described below) of a naturally occurring V_{H} domain from any animal species, in particular a species of mammal, such as from a human being, or by expression of a nucleic acid encoding such a camelized V_{H} domain; (6) by “camelisation” of a “domain antibody” or “Dab” as described by Ward et al (supra), or by expression of a nucleic acid encoding such a camelized V_{H} domain (7) using synthetic or semi-synthetic techniques for preparing proteins, polypeptides or other amino acid sequences; (8) by preparing a nucleic acid encoding a V_{HH} or a V_{H} using techniques for nucleic acid synthesis, followed by expression of the nucleic acid thus obtained; and/or (9) by any combination of the foregoing. Suitable methods and techniques for performing the foregoing will be clear to the skilled person based on the disclosure herein and for example include the methods and techniques described in more detail hereinbelow.

However, according to a specific embodiment, the heavy chain variable domains as disclosed herein do not have an amino acid sequence that is exactly the same as (i.e. as a degree of sequence identity of 100% with) the amino acid sequence of a naturally occurring V_{H} domain, such as the amino acid sequence of a naturally occurring V_{H} domain from a mammal, and in particular from a human being.

The term “affinity”, as used herein, refers to the degree to which a polypeptide, in particular an immunoglobulin, such as an antibody, or an immunoglobulin fragment, such as a VHH, binds to an antigen so as to shift the equilibrium of antigen and polypeptide toward the presence of a complex formed by their binding. Thus, for example, where an antigen and antibody (fragment) are combined in relatively equal concentration, an antibody (fragment) of high affinity will bind to the available antigen so as to shift the equilibrium toward high concentration of the resulting complex. The dissociation constant is commonly used to describe the affinity between the protein binding domain and the antigenic target. Typically, the dissociation constant is lower than 10⁻⁵ M. Preferably, the dissociation constant is lower than 10⁻⁶ M, more preferably, lower than 10⁻⁷ M. Most preferably, the dissociation constant is lower than 10⁻⁸ M.

The terms “specifically bind” and “specific binding”, as used herein, generally refers to the ability of a polypeptide, in particular an immunoglobulin, such as an antibody, or an immunoglobulin fragment, such as a VHH, to preferentially bind to a particular antigen that is present in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold).

Accordingly, an amino acid sequence as disclosed herein is said to “specifically bind to” a particular target when that amino acid sequence has affinity for, specificity for and/or is specifically directed against that target (or for at least one part or fragment thereof).

The “specificity” of an amino acid sequence as disclosed herein can be determined based on affinity and/or avidity.

An amino acid sequence as disclosed herein is said to be “specific for a first target antigen of interest as opposed to a second target antigen of interest” when it binds to the first target antigen of interest with an affinity that is at least 5 times, such as at least 10 times, such as at least 100 times, and preferably at least 1000 times higher than the affinity with which that amino acid sequence as disclosed herein binds to the second target antigen of interest. Accordingly, in certain embodiments, when an amino acid sequence as disclosed herein is said to be “specific for” a first target antigen of interest as opposed to a second target antigen of interest, it may specifically bind to (as defined herein) the first target antigen of interest, but not to the second target antigen of interest.

“Fungicidal activity”, as used herein, means to interfere with the harmful activity of a fungus, including but not limited to killing the fungus, inhibiting the growth or activity of the fungus, altering the behavior of the fungus, and repelling or attracting the fungus.

“Fungistatic activity”, as used herein, means to interfere with the harmful activity of a fungus, including but not limited to inhibiting the growth or activity of the fungus, altering the behavior of the fungus, and repelling or attracting the fungus.

“Culturing”, “cell culture”, “fermentation”, “fermenting” or “microbial fermentation” as used herein includes suspending the microbial cell in a broth or growth medium, providing sufficient nutrients including but not limited to one or more suitable carbon source (including glucose, sucrose, fructose, lactose, Avicel©, xylose, galactose, ethanol, methanol, or more complex carbon sources such as molasses or wort), nitrogen source (such as yeast extract, peptone or beef extract), trace element (such as iron, copper, magnesium, manganese or calcium), amino acid or salt (such as sodium chloride, magnesium chloride or natrium sulfate) or a suitable buffer (such as phosphate buffer, succinate buffer, HEPES buffer, MOPS buffer or Tris buffer). Optionally it includes one or more inducing agents driving expression of the compound of interest or a compound involved in the production of the compound of interest (such as lactose, avicel, IPTG, ethanol, methanol, sophorose or sophorolipids). If can also further involve the agitation of the culture media via for example stirring of purging to allow for adequate mixing and aeration. It can further involve different operational strategies such as batch cultivation, semi-continuous cultivation or continuous cultivation and different starvation or induction regimes according to the requirements of the microbial cell and to allow for an efficient production of the compound of interest or a compound involved in the production of the compound of interest. Alternatively, the microbial cell is grown on a solid substrate in an operational strategy commonly known as solid state fermentation.

Fermentation broth, culture media or cell culture media as used herein can mean the entirety of liquid or solid material of a fermentation or culture at any time during or after that fermentation or culture, including the liquid or solid material that results after optional steps taken to isolate the compound of interest, if produced. As such, the fermentation broth or culture media as defined herein includes the surroundings of the compound of interest after isolation of the compound of interest, during storage. Fermentation broth is also referred to herein as a culture medium or cell culture medium.

The culture medium may contain peptone. “Peptone” as used herein means a “protein hydrolysate”, which is any water-soluble mixture of polypeptides and amino acids formed by the partial hydrolysis of protein. More specifically “peptone” or “protein hydrolysate” are the water-soluble products derived from the partial hydrolysis of protein rich starting material which can be derived from plant, yeast, or animal sources. Typically, “peptone” or “protein hydrolysate” are produced by a protein hydrolysis process accomplished using strong acids, bases, or proteolytic enzymes. In more detail peptone or protein hydrolysates are produced by combining protein and demineralized water to form a thick suspension of protein material in large-capacity digestion vessels, which are stirred continuously throughout the hydrolysis process. For acid or basic hydrolysis, the temperature is adjusted, and the digestion material added to the vessel. For proteolytic digestion, the protein suspension is adjusted to the optimal pH and temperature for the specific enzyme or enzymes chosen for the hydrolysis. The desired degree of hydrolysis depends on the amount of enzyme, time for digestion, and control of pH and temperature. A typical “peptone” or “protein hydrolysate” may comprise about 25% polypeptides, about 30% free amino acids, about 20% carbohydrates, about 15% salts and trace metals and about 10% vitamins, organic acids, and organic nitrogen bases. Depending on the starting material “peptone” or “protein hydrolysate” can be completely free of animal-derived products and/or GMO products. For example, “Peptone” or “protein hydrolysate” can be produced using high quality pure protein as a starting material. Alternatively, “peptone” or “protein hydrolysate” can be produced by using soymeal as a starting material. When soymeal is used as a starting material this soymeal can be free of animal sources. This soymeal can furthermore be free of GMO material. This soymeal can be defatted soya. Alternatively, “peptone” or “protein hydrolysate” can be produced by using casein as a starting material. Alternatively, “peptone” or “protein hydrolysate” can be produce by using milk as a starting material. Alternatively, “peptone” or “protein hydrolysate” can be produce by using meat paste as a starting material. When meat paste is used as a starting material this meat paste can be for example from bovine or porcine origin. When meat paste is used as a starting material this meat paste can be derived from organs, such as harts or alternatively for example muscle tissue. Alternatively, “peptone” or “protein hydrolysate” can be produced using gelatin as a starting material. Alternatively, “peptone” or “protein hydrolysate” can be produced by using yeast as a starting material.

Accordingly, in some embodiments, the peptone is the product of partial hydrolysis of plant, animal or yeast protein.

In some embodiments, the peptone is produced by acid hydrolysis, by base hydrolysis or by enzymatic digestion.

In some embodiments, the peptone comprises at least about 5% polypeptides (weight/weight %). For example, in some embodiments the peptone comprises from about 5% to about 50% (weight/weight %) polypeptides.

In some embodiments, the peptone comprises at least about 5% (weight/weight %) free amino acids. For example, in some embodiments the peptone comprises from about 5% to about 50% (weight/weight %) free amino acids.

In some embodiments, the peptone comprises at least about 5% (weight/weight %) salts. For example, in some embodiments the peptone comprises from about 5% to about 20% (weight/weight %) salts.

In some embodiments, the peptone comprises at least about 5% (weight/weight %) carbohydrates. For example, in some embodiments the peptone comprises from about 5% to about 40% (weight/weight %) carbohydrates.

In some embodiments, the peptone comprises at least about 5% (weight/weight %) carbohydrates about 5% (weight/weight %) vitamins, organic acids, and organic nitrogen bases. For example, in some embodiments the peptone comprises from about 5% to about 20% (weight/weight %) vitamins, organic acids, and organic nitrogen bases.

In some embodiments, the peptone comprises from at least about 5% (weight/weight %) of polypeptides, at least about 5% (weight/weight %) free amino acids, at least about 5% (weight/weight %) salts, at least about 5% (weight/weight %) carbohydrates and at least about 5% (weight/weight %) in total of vitamins, organic acids, and organic nitrogen bases.

In some embodiments, the peptone comprises from about 15% to about 35% (weight/weight %) polypeptides, from about 20% to about 40% (weight/weight %) free amino acids, from about 10% to about 30% (weight/weight %) carbohydrates, from about 5% to about 25% (weight/weight %) salts, and from about 5% to about 15% (weight/weight %) in total of vitamins, organic acids, and organic nitrogen bases. Of course the skilled person will be aware the total amount cannot exceed 100% when all components are added together. The peptone may comprise additional components not specifically listed here.

In some embodiments, the peptone is free of animal derived products.

In some embodiments, the peptone is the product of partial hydrolysis of soymeal, casein, milk, meat, gelatine, or yeast.

Culturing in the presence of peptone means the cell culture medium comprise peptone. The peptone may be present at any suitable concentration. For example, in some embodiments the peptone concentration may be from about 1 g/L to about 100 g/L, for example from about 10 g/L to about 80 g/L, for example about 20 g/L, about 30 g/L, about 40 g/L, about 50 g/L, about 60 g/L, or about 70 g/L.

The cell culture medium used for culture of the microbial host cell may already comprise peptone. Alternatively, the cell culture medium may be modified to include peptone. For example, peptone may be added to the cell culture medium at any suitable time during the culturing of the microbial cell. For example, in embodiments where the compound of interest is encoded by a nucleotide that is operably linked to an inducible promoter, the peptone may be added to the cell culture medium in the fermentation chamber at the same time as or shortly after expression of the compound of interest is induced. Alternatively, the peptone may be added to the cell culture medium in the fermentation chamber before induction of expression of the compound of interest.

In embodiments where the cell culture medium does not already comprise peptone and this must be added to the cell culture medium, this may be added to the cell culture medium before adding the cell culture medium to the fermentation chamber. Alternatively, the peptone may be added to the fermentation chamber separately, preferably after the cell culture medium is added to the fermentation chamber.

“Isolating the compound of interest” or “isolating the protein of interest” is an optional step or series of steps taking the cell culture media or fermentation broth as an input and increasing the amount of the compound of interest relative to the amount of culture media or fermentation broth. Isolating the compound of interest may alternatively or additionally comprises obtaining or removing the compound of interest form the culture media or fermentation broth. Isolating the compound of interest can involve the use of one or multiple combinations of techniques well known in the art, such as precipitation, centrifugation, sedimentation, filtration, diafiltration, affinity purification, size exclusion chromatography and/or ion exchange chromatography. In some embodiments, isolating the compound of interest may comprise a step of lysing the microbial cells to release the compound of interest, for example if the compound of interest is not secreted by the microbial cells, or at least is not secreted by the microbial cells to a significant enough degree. Isolating the compound of interest may be followed by formulation of the compound of interest into an agrochemical or pharmaceutical composition.

“Agrochemical”, “agrochemically” or “agrochemically suitable” as used herein, means suitable for use in the agrochemical industry (including agriculture, horticulture, floriculture and home and garden uses), but also products intended for non-crop related uses such as public health/pest control operator uses to control undesirable insects and rodents, household uses, such as household fungicides and insecticides and agents, for protecting plants or parts of plants, crops, bulbs, tubers, fruits (e.g. from harmful organisms, diseases or pests); for controlling, preferably promoting or increasing, the growth of plants; and/or for promoting the yield of plants, crops or the parts of plants that are harvested (e.g. its fruits, flowers, seeds etc.). Examples of such substances will be clear to the skilled person and may for example include compounds that are active as insecticides (e.g. contact insecticides or systemic insecticides, including insecticides for household use), herbicides (e.g. contact herbicides or systemic herbicides, including herbicides for household use), fungicides (e.g. contact fungicides or systemic fungicides, including fungicides for household use), nematicides (e.g. contact nematicides or systemic nematicides, including nematicides for household use) and other pesticides or biocides (for example agents for killing insects or snails); as well as fertilizers; growth regulators such as plant hormones; micro-nutrients, safeners, pheromones; repellants; insect baits; and/or active principles that are used to modulate (i.e. increase, decrease, inhibit, enhance and/or trigger) gene expression (and/or other biological or biochemical processes) in or by the targeted plant (e.g. the plant to be protected or the plant to be controlled), such as nucleic acids (e.g., single stranded or double stranded RNA, as for example used in the context of RNAi technology) and other factors, proteins, chemicals, etc. known per se for this purpose, etc. Examples of such agrochemicals will be clear to the skilled person; and for example include, without limitation: glyphosate, paraquat, metolachlor, acetochlor, mesotrione, 2,4-D,atrazine, glufosinate, sulfosate, fenoxaprop, pendimethalin, picloram, trifluralin, bromoxynil, clodinafop, fluroxypyr, nicosulfuron, bensulfuron, imazetapyr, dicamba, imidacloprid, thiamethoxam, fipronil, chlorpyrifos, deltamethrin, lambda-cyhalotrin, endosulfan, methamidophos, carbofuran, clothianidin, cypermethrin, abamectin, diflufenican, spinosad, indoxacarb, bifenthrin, tefluthrin, azoxystrobin, thiamethoxam, tebuconazole, mancozeb, cyazofamid, fluazinam, pyraclostrobin, epoxiconazole, chlorothalonil, copper fungicides, trifloxystrobin, prothioconazole, difenoconazole, carbendazim, propiconazole, thiophanate, sulphur, boscalid and other known agrochemicals or any suitable combination(s) thereof.

An “agrochemical composition”, as used herein means a composition for agrochemical use, as further defined, comprising at least one active substance, optionally with one or more additives (for example one or more additives favoring optimal dispersion, atomization, deposition, leaf wetting, distribution, retention and/or uptake of agrochemicals). It will become clear from the further description herein that an agrochemical composition as used herein includes biological control agents or biological pesticides (including but not limited to biological biocidal, biostatic, fungistatic and fungicidal agents) and these terms will be interchangeably used in the present application. Accordingly, an agrochemical composition as used herein includes compositions comprising at least one biological molecule as an active ingredient, substance or principle for controlling pests in plants or in other agro-related settings (such for example in soil). Non-limiting examples of biological molecules being used as active principles in the agrochemical compositions disclosed herein are proteins (including antibodies and fragments thereof, such as but not limited to heavy chain variable domain fragments of antibodies, including VHH's), nucleic acid sequences, (poly-) saccharides, lipids, vitamins, hormones glycolipids, sterols, and glycerolipids. As a non-limiting example, the additives in the agrochemical compositions disclosed herein may include but are not limited to excipients, diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents. The compound of interest may be formulated with one or more such components when preparing an agrochemical composition. For example, the compound of interest may be formulated with one or more additives, for example one or more agrochemically acceptable excipients.

A “pharmaceutical composition”, “pharmaceutically” or “pharmaceutically suitable” as used herein means a composition for medical use. For example, the composition may be suitable for injection or infusion which can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin. The compound of interest may be formulated with one or more such components when preparing a pharmaceutical composition. For example, the compound of interest may be formulated with one or more additives, for example one or more pharmaceutically acceptable excipients.

A modification, modified, edits, editing, genome editing, genome editing within a cell, or similar term as used herein means the alteration of the genetic material or the DNA of a cell. Editing the genetic material of a cell in light of this invention is a directed method (i.e. modifications are not introduced at random). Modifications or edits may comprise point mutations, SNPs, frame-shift mutations, indel-mutations, deletions, insertions or other forms of alteration of the genetic material. For instance, inactivation of a specific target or gene can be achieved by introducing a frame-shift into the coding region of said gene, specific point mutations such as introduction of a premature stop codon also has the capacity to inactivate a certain gene or target. Alternatively, specific point mutations can be made that for example change one or more amino acids so to alter the protein sequence of the target (be it to inactivate, alter the protein or provide a gain of function). Editing the genetic material of a cell also includes deleting portions or the entirety of a target gene. According to the methods of the invention, these deletions can be made very specific as to remove only a subsection or specific domain of a protein but leave the remainder of the protein intact. Deletions can also be so constructed as to have a clean deletion of the target gene leaving no marker or so-called scar behind in the genome. Alternatively, to prevent polar effects, a deletion can also be made such that a small peptide remains that has no biological function but serves to prevent read-through of the transcription and translation machinery. One alternative to this is to include stop-codons into the site of the deleted gene. Multiple stop codons can be included for example a set of three stop codons that are each in a different frame. The current invention further provides methods to insert proteins of interest into the genome of the cell. For example, a protein of interest can be introduced in one or more sites of the genome, for example, downstream of a particular promoter such as the cbh1 promoter. Insertions can also involve the formation of fusion proteins, such as the fusion of a protein of interest to a signal peptide such as the signal peptide of the cbh1 gene. Or the insertions can involve the insertion or the fusion of a fluorescent marker (such as the green fluorescent protein) into an operon or to a gene of interest. Genomic modifications can also include replacements whereby a certain target gene is replaced in its entirety (or partially) by a gene of interest or a construct of interest. Note that an insertion does not necessarily need be a gene of interest encoding a protein of interest but can also be for instance a promoter or a genetic construct useful for engineering purposes (such as frt or lox sites). In preferred embodiments the method of the invention is used to provide deletions or inactivation of proteins such as proteases which can hamper with the stability or production of a protein of interest. In another preferred embodiment, the method of the invention is used to insert one or more copies of a protein of interest into the genome of a cell.

The methods of the invention may be employed for the modification or editing of the genome of a cell. In a more preferred embodiment, the cell is a microbial cell. In an even more preferred embodiment, the cell is a fungus. In a preferred embodiment the cell is a filamentous fungus. The fungi may preferably be from the division Ascomycota, subdivision Pezizomycotina. In some embodiments, the fungi may preferably from the Class Sordariomycetes, optionally the Subclass Hypocreomycetidae. In some embodiments, the fungi may be from an Order selected from the group consisting of Hypocreales, Microascales, Eurotiales, Onygenales and Sordariales. In some embodiments, the fungi may be from a Family selected from the group consisting of Hypocreaceae, Nectriaceae, Clavicipitaceae and Microascaceae. In some more specific embodiments, the fungus may be from a Genus selected from the group consisting of Trichoderma (anamorph of Hypocrea), Myceliophthora, Fusarium, Gibberella, Nectria, Stachybotrys, Claviceps, Metarhizium, Villosiclava, Ophiocordyceps, Cephalosporium, Neurospora, Rasamsonia and Scedosporium. In some further and more specific embodiments, the fungi may be selected from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Neurospora crassa, Rasamsonia emersoniim, Acremonium (Cephalosporium) chrysogenum, Scedosporium apiospermum, Aspergillus niger, A. awamori, A. oryzae, A. nidulans, Chrysosporium lucknowense, Thermothelomyces thermophilus, Myceliophthora thermophila, Myceliophthora heterothallica, Thermothelomyces heterothallica, Humicola insolens, and Humicola grisea, most preferably Trichoderma reesei or Myceliophthora heterothallica. If the host cell is a Trichoderma reesei cell, it may be selected from the following group of Trichoderma reesei strains obtainable from public collections: QM6a, ATCC13631; RutC-30, ATCC56765; QM9414, ATCC26921, RL-P37 and derivatives thereof. If the host cell is a Myceliophthora heterothallica, it may be selected from the following group of Myceliophthora heterothallica or Thermothelomyces thermophilus strains: CBS 131.65, CBS 203.75, CBS 202.75, CBS 375.69, CBS 663.74 and derivatives thereof. If the host cell is a Myceliophthora thermophila it may be selected from the following group of Myceliophthora thermophila strains ATCC42464, ATCC26915, ATCC48104, ATCC34628, Thermothelomyces heterothallica C1, Thermothelomyces thermophilus M77 and derivatives thereof. If the host cell is an Aspergillus nidulans it may be selected from the following group of Aspergillus nidulans strains: FGSC A4 (Glasgow wild-type), GR5 (FGSC A773), TN02A3 (FGSC A1149), TN02A25, (FGSC A1147), ATCC 38163, ATCC 10074 and derivatives thereof.

The present invention will now be illustrated by way of the following non-limiting Examples.

### EXAMPLES

### Example 1: Modification of Filamentous Fungal Cells Mediated by CRISPR-Cas

### 1.1 Guide-RNA Design and Preparation

Guide-RNA sequences were selected and designed in silico using Cas-Designer and Cas-OFFinder (http://www.rgenome.net/) to reduce off-target cleavage. For each target locus, two guide-RNAs were selected located upstream and downstream of the target locus. The selected guide-RNAs were synthesized by a commercial supplier. The lyophilised guide-RNAs were resuspended in TE Buffer at pH 7.5 to a concentration of 100 μM. Recombinant Cas12a enzyme was purchased from New England BioLabs. For illustration purposes several strategies were tested. The Trichoderma are1 gene was deleted using guide RNAs according to SEQ ID Nos: 19-20; cbh1 gene was targeted for introducing a compound of interest using guide RNAs according to SEQ ID Nos: 21-22; genes pep5, sep1, gap2, pep1 and gap1 encoding proteases from Trichoderma reesei were deleted using the guide RNAs according to respectively SEQ ID Nos: 23-24, SEQ ID Nos: 24-26, SEQ ID Nos: 27-28, SEQ ID Nos: 29-30 and SEQ ID Nos:31-32. Note that the here listed guide-RNAs are grouped per pair as used in the corresponding pair of RNPs. In this example, the guide RNAs where so designed so that they are capable to interact with the Cpf1 endonuclease to form a RNP.

### 1.2 Construction of Donor-DNAs

Donor-DNA was obtained via gene synthesis from a commercial manufacturer and thereafter cloned into a standard plasmid backbone, such as used here the TOPO cloning vector (Thermo Fisher Scientific). This construct can be used as a template in a PCR to obtain sufficient DNA material for successful transformation into fungal cells. The are1 gene was deleted using a traditional approach where the donor-DNA 5′-end and 3′-end flank a HygB selection cassette according to SEQ ID NO: 33. A construct encoding the VHH according to SEQ ID NO: 1 was introduced into the cbh1 locus using donor-DNA according to SEQ ID NO: 34. For the deletion of the pep5, sep1, gap2, pep1 and gap1 genes the donor-DNA constructs according to respectively SEQ ID NOs: 35 to 39 were used. The PCR was performed employing the Phusion High-Fidelity PCR kit (Thermo Fisher Scientific). Subsequently, the PCR products were purified using the Wizard DNA Clean-Up System (Promega) according to the manufacturer's instructions.

### 1.3 In Vitro RNP Complex Formation

The cas12a Ribonucleoprotein was assembled by mixing 100 μM cas12a with 100 μM guide-RNA in a 0.2 mL PCR reaction tube. The mixture was incubated at room temperature for 20 min to allow complex formation. Each RNP complex was formed separately.

### 1.4 Construction of Autonomously Replicating Plasmids for Selection Marker Recycling

The autonomously replicating plasmids pAMA1-hygB (FIG. 2 and SEQ ID NO: 16) and pAMA1-BleoR (FIG. 3 and SEQ ID NO: 17) were constructed as follows: the pAMA1-hygB includes the 5.5 Kb AMA1 sequence of the autonomously replicating origin from Aspergillus nidulans sequence (Aleksenko and Clutterbuck, 1996) and a selection marker expression cassette comprising the hygB gene (encoding hygromycin B phosphotransferase), under the control of the oliC promoter and the trpC terminator of Aspergillus nidulans (PoliC-hph-TtrpC). pAMA1-hygB was obtained via gene synthesis. The construction of selection marker pAMA1-BleoR was obtained by replacing the hygB antibiotic resistance gene with the BleoR antibiotic resistance gene by standard restriction enzyme and ligation.

### 1.5 Genetic Transformation of Fungal Cells

For each transformation of Trichoderma, 200 μL protoplasts (˜1×10⁸ protoplasts/ml), up to 20 μL donor-DNA construct (containing 1-4 μg of each donor-DNA construct as necessary), 2 μL of each Cas12a RNP complex (100 μM of each pair of guide-RNAs complexed with Cpf1 as described above), 1 μg pAMA1-hygB or pAMA1-BleoR and 50 μL 25% PEG 6000 buffer were mixed in a 50 ml Falcon tube. Transformation was performed according to a standard poly-ethylene glycol (PEG) mediated transformation method as described previously [Penttils M., Nevalainen, H., Rstt, M., Salminen, E., Knowles, J., 1987. Gene 61, 155-64]. Briefly, the samples were incubated for 30 minutes on ice, then incubated with 2 ml of 25% PEG 6000 solution for 5 minutes. Samples were then mixed with STC buffer and incubated for 5 minutes at room temperature. Transformation mixtures were plated on PDA plates supplemented with 1.2 M sorbitol in the presence of 100 μg mL-1 hygromycin B or 25 μg mL-1 phleomycin, and incubated at 37° C. for 24 h and then for 3 days at 28° C. To confirm the integration of the expression cassettes, colony PCR was performed under standard PCR conditions with sequence-specific PCR primers.

Transformation of Myceliophthora with the target DNA is achieved by mixing 200 μL protoplasts, up to 15 μL donor DNA (1-4 μg per target for multiplex-targeting), 2 μL of each Cas12a RNP complex (100 μM), 1 μg pAMA1-hyg or pAMA1-BleoR selection marker plasmids, 1×STC, and 60% PEG4000 buffer, and co-introduced into the fungal strains using a standard poly-ethylene glycol (PEG) mediated transformation method as described previously [dos Santos Gomes et al. 2019]. Successful transformants are selected on minimal medium containing AspA+N, 2 mM MgSO4, 0.1% trace elements, 0.1% casamino acids, 670 mM sucrose, 1% D-glucose, and 1.5% agar with 100 μg mL-1 hygromycin B or 25 μg mL-1 phleomycin as the selective agent. The plates are incubated at 37° C. for 4 days until colonies could be picked to secondary selection plates.

### 1.6 High-Throughput-Based Transformation

For high-throughput Trichoderma transformation protocol in 48 deep-well microtiter plates the following protocol was developed. A solution of 50 μL of protoplasts (1×10⁵/mL) was transferred in 48 deep-well plates and mixed with up to 18 μL donor DNA (1 μg of each donor-DNA construct), 1 μL of each of the pair of the Cas12a RNP complex (100 μM of each RNP), 0.5 μg pAMA1-hygB or pAMA1-BleoR selection marker plasmids, and 20 μL of 25% PEG6000 solution. The deep-well plate was incubated for 10 min at 4° C. after which an additional 500 μL of PEG6000 solution was added. Samples were incubated for 2 min at room temperature after which the wells were filled with a final volume of 1 mL STC buffer. Subsequently, the cell suspension was transferred to 15 ml Falcon tubes containing 10 mL molten top agar, mixed slowly, and plated on hygromycin or phleomycin selection plates and incubated for 3 days at 37° C.

The MTP transformation protocol for Myceliophthora is implemented as well in 48 deep-well microtiter plates. A solution of 50 μL of protoplasts (1×10⁵/mL) is transferred in 48 deep-well plates and mixed with up to 10 μL donor DNA (1 μg per target for multiplex-targeting), 1 μL of each of the pair of the Cas12a RNP complex (100 μM), 0.5 μg AMA1-hygB or AMA1-BleoR selection marker plasmids, 55 μL of STC-buffer and 20 μL of PEG4000 solution. The deep-well plates are incubated for 20 min at 4° C. after which an additional 114 μL of PEG4000 solution is added. Samples are incubated for an additional 5 min at room temperature after which the wells are filled with a final volume of 226 μL STC buffer. Subsequently, the plate was centrifuged at 2500 rpm for 10 min at 4° C. and subsequently, the supernatant is removed, and the pellet is resuspended in a small leftover amount. Samples are plated on selection plates and incubated for 3 days at 37° C.

### 1.7 Screening of Cells

The antibiotic-resistant colonies and parental host cells were grown in YPD liquid medium (10 g/L yeast extract, 20 g/L peptone, and 20 g/L glucose). Genomic DNA was extracted from colonies using Phire Plant Direct PCR Kit (Thermo Fisher Scientific) according to the manufacturer's instructions. The resulting genomic DNA sample was diluted into 10 μl water, mycelium was removed by centrifugation, and the supernatant was used as a template in subsequent PCR.

Oligonucleotides were designed outside the flanking regions of the target locus to identify the possible integration of the donor cassette or deletion. The expected size of the deletion or insertion was variable and target depending. Positive transformants confirmed by colony PCR were further purified to obtain single spore isolations, followed by two or three rounds of subculturing under nonselective conditions to remove the AMA1-based plasmid from transformants.

### 1.8 General Procedures for Performing a Fermentation

Fermenters are filled with medium with similar characteristics as described in Table 4, or in a defined medium (Trire) containing ammonium sulphate (NH₄)₂SO₄ and peptone using either lactose or sophorose as inducers. Calibration of the Dissolved oxygen (DO) levels is performed at around 37° C., 400 rpm and 60 sL/h of aeration. The pH of the medium in the fermenter is adjusted to around 4 or 5 before being inoculated in the fermenter.

Fermenters are inoculated with around 0.5%-10% inoculum density in 1980 ml medium. Incubation at around 28° C.; 1200 rpm and 60 sL/h aeration. DO lower limit at 50%. DO cascade output set as 0-40% 1200-1400 rpm of stirrer, 40-100%, 100-200 tl/h of aeration. Antifoam is dissolved as 10× in water. Ammonium hydroxide 12.5% as base. Induction with for instance lactose 20% is generally initiated after a p02 spike. The feed rate is set at approximately 9 ml/h (4.5 ml/L·h).

### 1.9 Targeting Efficiency of CRISPR-Mediated Single Gene Deletion Using a Pair of RNPs

A single deletion was made with a donor-DNA construct including a selection marker HygB (SEQ ID NO: 33) and by using a pair of RNPs formed by guide-RNAs according to SEQ ID Nos: 19-20. Corresponding RNPs and donor DNA were transformed in the absence of an AMA1-based plasmid. This transformation yielded 5 successful are1 deletions out of 6 screened colonies. Note that this is the only example where the HygB cassette was included in the donor-DNA construct. Following results used one of the pAMA1-based plasmids as described above and thus did not contain a selectable marker in the donor-DNA construct.

Inserting a refVHH expression cassette using a donor-DNA construct and the RNPs formed with guide-RNAs according to SEQ ID NOs: 21-22, yielded 18 successful integrations out of 26 screened colonies. Comparably, when using the high-throughput-based transformation as described above 4 out of 5 screened colonies successfully integrated the VHH expression cassette according to SEQ ID NO: 34.

A double deletion of pep1 and gap1, using donor-DNA constructs according to SEQ ID NOs: 38 and 39 with corresponding pairs of RNPs using the guide RNAs according to respectively SEQ ID NOs: 29-30 and SEQ ID NOs: 31-32, yielded 100% double transformants containing both deletions

A triple deletion, deleting pep5, sep1 and gap2 genes using the donor-DNA constructs according to respectively SEQ ID Nos: 35-37 and corresponding pairs of RNPs including guide RNAs according to SEQ ID Nos: 23-24, SEQ ID Nos: 25-26 and SEQ ID Nos: 27-28 yielded, out of 22 clones, 72.7% clones with all three deletions, 22.7% clones with 2 deletions and 4.5% clones with one deletion. Zero clones contained no deletions.

Similarly, a double deletion made by the high-throughput-based transformation method, yielded 100% efficiency of clones containing both deletions out of 12 clones screened.

This demonstrates that the method of the invention is highly efficient for producing modified fungal cells with multiple modifications in a single round of transformation. The efficiencies observed for recovery of cells with the intended number of modifications were higher than for recovery of cells having none or less than the intended number of modifications.

In all cases above using the pAMA1-based plasmids, the transformants were streaked two times on non-selective plates to remove the antibiotic selection marker. The strains regained sensitivity to the respective antibiotic, indicating that the selection marker was successfully removed and can be used again in the next genetic engineering procedure.

1.10 Multiplexing Genome Deletions with Different 5′-End and 3′-End Sequence Lengths

A double deletion of pep1 and gap1, using donor-DNA constructs according to SEQ ID NOs: 38 and 39 with corresponding pairs of RNPs using the guide RNAs according to respectively SEQ ID NOs: 29-30 and SEQ ID NOs: 31-32, yielded 100% double transformants containing both deletions. Since the cell was co-transformed with an AMA1-based plasmid comprising the selection marker as disclosed herein, it was, it was possible to continue to the next round of genetic modifications without marker plasmid removal by using an AMA1-based plasmid with a different selection marker.

In this second round, the triple deletion, including pep5, sep1, and gap2 genes using the donor-DNA constructs according to respectively SEQ ID Nos: 35-37 and corresponding pairs of RNPs including guide RNAs according to SEQ ID Nos: 23-24, SEQ ID Nos: 25-26 and SEQ ID Nos: 27-28 yielded, out of 22 clones, 72.7% clones with all three deletions, 22.7% clones with 2 deletions and 4.5% clones with one deletion. Zero clones contained no deletions. After a transfer of the clones to a medium without antibiotics, the deletion strains did not show resistance to hygromycin or phleomycin, which indicated that the deleted strains were marker-free and ready for a third round of genetic modifications.

This demonstrates that the method of the invention is highly efficient for producing modified fungal cells with multiple modifications in a single round of transformation. The efficiencies observed for the recovery of cells with the intended number of modifications were higher than for the recovery of cells having none or less than the intended number of modifications. Furthermore, single rounds of transformation can be successively performed without the need of extra genetic engineering steps to remove the AMA1-based plasmids.

In all cases above using the pAMA1-based plasmids, the transformants were streaked two times on non-selective plates to remove the antibiotic selection marker. The strains regained sensitivity to the respective antibiotic, indicating that the selection marker was successfully removed and can be used again in the next genetic engineering procedure.

Whereas HDR requires long homology 5′-end and 3′-end sequences (˜900 to 2,000 bp) for precise deletion or insertion of the repair template, several flank homology lengths were chosen to determine the efficiency of knockouts or knock-ins by using short flanking regions of the target genes. Under this approach, 700 bp, 350 bp, 200 bp, 100 bp, and 50 bp were evaluated to repair the template by short microhomology and the high-throughput-based transformation method. Primers were designed to amplify the deletion cassettes with varying 5′-end and 3′-end sequence lengths (See Table 1). All expression cassettes were amplified from their respective plasmids as described in Example 1.2. As a proof of concept, two target genes were chosen: gap1 (with corresponding amplification primers with SEQ ID NO: 40 to 49) and slp1 (with corresponding amplification primers with SEQ ID NO: 50 to 59).

TABLE 1
List of the primers used for the amplification of the deletion cassettes.

| SEQ ID NO | Primer name | Sequence |
|---|---|---|
| 40 | Tr_gap1_50bp_Fw | AAATATTACCCTCTGCCTCTTCTTGAGT |
| 41 | Tr_gap1_50bp_Rv | CTGCTACATGTTGCCGGG |
| 42 | Tr_gap1_100bp_Fw | TGTGAGTAGTTGGGGAAGGGAT |
| 43 | Tr_gap1_100bp_Rv | TTTGTATCATCAGTATGTATAGACTCAAAAATTTGTGG |
| 44 | Tr_gap1_200bp_Fw | CTGGATGTTGACGCCAGC |
| 45 | Tr_gap1_200bp_Rv | TAATATTGCACACGCCCTTGCC |
| 46 | Tr_gap1_350bp_Fw | TAGCATGTATCGCCCAATTTAGTTTGT |
| 47 | Tr_gap1_350bp_Rv | ACCCCTTTACGCTATGATCTTATATCTTCT |
| 48 | Tr_gap1_700bp_Fw | GCAAATCAGGAAGGTGATTAGTCCCC |
| 49 | Tr_gap1_700bp_Rv | TACGGAAGAAAGATGCGAATTTCATATACCC |
| 50 | Tr_slp1_50bp_Fw | AGAACAGCAGCAGCGCT |
| 51 | Tr_slp1_50bp_Rv | CGCCATTAGATGTTTTATACCTGCTTACA |
| 52 | Tr_slp1_100bp_Fw | ACTGGGCAGCCTCCC |
| 53 | Tr_slp1_100bp_Rv | CTTCCTGCAATGAGCAGCTCA |
| 54 | Tr_slp1_200bp_Fw | CAGCGAAGATGGTACTCCCCT |
| 55 | Tr_slp1_200bp_Rv | GCGATGGATTTTGGAGACGGT |
| 56 | Tr_slp1_350bp_Fw | CTAGACATGGGTCGAAAGTGGC |
| 57 | Tr_slp1_350bp_Rv | CAAGATCGGTGGGGGTGC |
| 58 | Tr_slp1_700bp_Fw | AACGTTCCGCATCGGCC |
| 59 | Tr_slp1_700bp_Rv | CGAGGGGGCAGAGTTGC |

The amplified PCR products were subsequently pooled together for each amplified cassette, cleaned with the Wizard® SV Gel and PCR Clean Up System, and stored at −20° C.

For the transformation, a Trichoderma strain was chosen that did not contain the deletions of gap1 and slp1. The high-throughput transformation protocol described in example 1.6 was used in this example. The transformation was performed and after incubation, transformants were visible and subjected to colony purification. To verify if the deletions succeeded, PCR screening was performed.

The screening revealed that both genes were successfully deleted with all the different deletion cassettes that were generated. Upstream and downstream flanks of up to 50 bp were successfully used to delete the respective genes. To see if there is a correlation between the 5′-end and 3′-end sequence lengths and the efficiency of the deletions, an overview was made which is summarised in Table 2 and 3.

The efficiencies calculated for the deletion of gap1 and slp1 using 50 bp 5′-end and 3′-end sequences reached up to 80% efficiency, indicating the positive impact of this HTP method to delete simultaneously several genes with a minimal screening effort of less than 10 screened transformants.

TABLE 2
Overview of the PCR screening for the deletion of
gap1 with different 5′-end and 3′-end
sequence lengths. Plus symbol indicates the successful
deletion of the gene, negative symbol indicates
that the deletion failed. If double bands (heterokaryon)
were seen, the deletion was not counted as
successful.

| | gap1- | gap1- | gap1- | gap1- | gap1- |
|---|---|---|---|---|---|
| Colony | 50 bp | 100 bp | 200 bp | 350 bp | 700 bp |
| 1 | + | + | + | + | + |
| 2 | + | + | − | + | − |
| 3 | + | + | + | + | + |
| 4 | + | − | + | + | + |
| 5 | + | + | + | + | + |
| 6 | + | + | + | − | + |
| Efficiency | 100% | 83.3% | 83.3% | 83.3% | 83.3% |

TABLE 3
Overview of the PCR screening for the deletion
of slp1 with different 5′-end and 3′-end
sequence lengths. Plus symbol indicates the successful
deletion of the gene, negative symbol indicates
that the deletion failed. If double bands (heterokaryon)
were seen, the deletion was not counted as
successful.

| | slp1- | slp1- | slp1- | slp1- | slp1- |
|---|---|---|---|---|---|
| Colony | 50 bp | 100 bp | 200 bp | 350 bp | 700 bp |
| 1 | + | Double | + | Double | Double |
| 2 | − | Double | + | + | + |
| 3 | + | − | + | − | Double |
| 4 | + | + | + | + | Double |
| 5 | + | Double | + | + | + |
| 6 | + | + | + | + | + |
| Efficiency | 83.3% | 33.3% | 100% | 66.7% | 50% |

### 1.11 Targeting Efficiency of Multiplex Target Insertions

To test whether this CRISPR-Cas system could efficiently target multiple simultaneous insertions of expression cassettes by using the high-throughput-based transformation method of example 1.6, it was explored to target three different loci simultaneously with expression cassettes of up to 4,000 bp. To generate a recombinant protein expression cassette, a codon-optimized version of a polynucleotide encoding a VHH was fused with the cellobiohydrolase I (CBHI) signal peptide coding sequence under the control of the cbhl promoter sequence, which was synthesized. Alternatively, the catalytic domain fragment of cbhl was fused with the intact codon-optimized version of the polynucleotide encoding the compound of interest, including the KexB protease cleavage site to release the recombinant protein and Cbhl carrier protein separately during protein secretion. The same expression cassette mentioned was readapted for its targeted integration. The expression cassettes containing the target protein were flanked with 5′ and 3′ DNA homologous regions (˜400 bp each) of the target loci, which resulted in the coding region of the target loci being replaced by the expression cassette.

A one-step co-transformation containing the selection marker AMA1-BleoR, the donor DNAs (three recombinant protein expression cassettes to target the different loci simultaneously), and the CRISPR-Cas system using the pooled gRNAS targeting all three target loci were mixed and transformed according to Examples 1.3 and 1.6.

After transformation, protoplasts were incubated at 28° C. for 4-6 days on selection plates. Then only less than 10 transformants were picked randomly for PCR analysis. To confirm the integration of the expression cassettes, colony PCR was performed under standard PCR conditions with sequence-specific PCR primers.

From 8 transformants, 37.5% showed triple integration, 25% double integration, and 37.5% displayed single integrations in the three loci. The resulting transformants were streaked two times on non-selective plates to remove the antibiotic selection marker, showing that all of them regained sensitivity to the respective antibiotic and indicating that the selection marker was successfully removed to be used in the next genetic engineering procedure. Overall, these results demonstrate that this CRISPR-Cas method can efficiently mediate multiplex gene insertions in a single transformation with a cheaper, and effortless screening of a few transformants.

Similar to the approach in example 1.10, the integration efficiency of VHH expression cassettes with different flank homology lengths of either 100 bp, 250 bp, 400 bp, or 600 bp, in the cbh1 locus of Trichoderma reesei was evaluated. The genetic manipulation was performed by co-delivery of the PCR products with different homology lengths, the selection marker AMA1-BleoR, and the pooled gRNAS targeting the cbh1 locus.

The integration efficiency in generating VHH-expressing strains is summarized in Table 4. The integration efficiency was significantly high for all the flank lengths tested.

TABLE 4
Overview of the PCR screening for the insertion of
VHH expression cassette with different
flank homology lengths into cbh1 locus.

| Flank | No. transformants | Not | Insertion |
|---|---|---|---|
| lengths | evaluated | inserted | efficiency |
| 600 bp | 27 | 0 | 100.0% |
| 400 bp | 23 | 1 | 95.6% |
| 250 bp | 10 | 2 | 80.0% |
| 100 bp | 25 | 5 | 80.0% |

TABLE 5
Sequences referred to herein

| | | |
|---|---|---|
| SEQ | 1 | DVQLVESGGGLVQAGGSLRLSCAASRSIFSINAMDWYRQAPGKQREWVAGITRGGTTKYADSVKGRFTISRDNAKKKVYLQMN |
| ID | | SLKPEDTAVYYCNVLRGEQPWTRDYWGQGTQVTVSS |
| NO | | |
| SEQ | 2 | QVQLVESGGGLVQAGGSLRLSCAASRSIFSINAMDWYRQAPGKQREWVAGITRGGTTKYADSVKGRFTISRDNAKKKVYLQMN |
| ID | | SLKPEDTAVYYCNVLRGEQPWTRDYWGQGTQVTVSS |
| NO | | |
| SEQ | 3 | RSIFSINAMD |
| ID | | |
| NO | | |
| SEQ | 4 | GITRGGTTK |
| ID | | |
| NO | | |
| SEQ | 5 | LRGEQPWTRDY |
| ID | | |
| NO | | |
| SEQ | 6 | QVQLQESGGGLVQAGGSLRLSCAASGTIFRPTAMGWYRQAPGKERELVATITTGGSTKYADSVKGRFTISRGNAKNTVYLQMSS |
| ID | | LKPEDTAVYYCNAQWGVRTRDYWGQGTQVTVSS |
| NO | | |
| SEQ | 7 | GTIFRPTAMG |
| ID | | |
| NO | | |
| SEQ | 8 | TITTGGSTK |
| ID | | |
| NO | | |
| SEQ | 9 | QWGVRTRDY |
| ID | | |
| NO | | |
| SEQ | 10 | QVQLQESGGGLVQAGDSLRLSCAASISDRAFSRHVMGWFRQPPGKEREFVAAIGWTGRRTYYADSVKGRFTISRDNAMNTVYL |
| ID | | QMNSLKPEDTAVYYCAASHFYSVSFEINDYDYWGQGTQVTVSS |
| NO | | |
| SEQ | 11 | ISDRAFSRHV |
| ID | | |
| NO | | |
| SEQ | 12 | AIGWTGRRTY |
| ID | | |
| NO | | |
| SEQ | 13 | SHFYSVSFEINDYDY |
| ID | | |
| NO | | |
| SEQ | 14 | DVQLQESGGGLVQAGGSLRLSCAASGTIFRPTAMGWYRQAPGKERELVATITTGGSTKYADSVKGRFTISRGNAKNTVYLQMSS |
| ID | | LKPEDTAVYYCNAQWGVRTRDYWGQGTQVTVSS |
| NO | | |
| SEQ | 15 | DVQLQESGGGLVQAGDSLRLSCAASISDRAFSRHVMGWFRQPPGKEREFVAAIGWTGRRTYYADSVKGRFTISRDNAMNTVYL |
| ID | | QMNSLKPEDTAVYYCAASHFYSVSFEINDYDYWGQGTQVTVSS |
| NO | | |
| SEQ | 16 | CTAAATTGTAAGCGTTAATATTTTGTTAAAATTCGCGTTAAATTTTTGTTAAATCAGCTCATTTTTTAACCAATAGGCCGAAATC |
| ID | | GGCAAAATCCCTTATAAATCAAAAGAATAGACCGAGATAGGGTTGAGTGGCCGCTACAGGGCGCTCCCATTCGCCATTCAG |
| NO | | GCTGCGCAACTGTTGGGAAGGGCGTTTCGGTGCGGGCCTCTTCGCTATTACGCCAGCTGGCGAAAGGGGGATGTGCTGCA |
| | | AGGCGATTAAGTTGGGTAACGCCAGGGTTTTCCCAGTCACGACGTTGTAAAACGACGGCCAGTGAGCGCGACGTAATACG |
| | | ACTCACTATAGGGCGAATTGGCGGAAGGCCGTCAAGGCCGCATAGCTTATTTTTTGTATACTGTTTTGTGATAGCACGAAGT |
| | | TTTTCCACGGTATCTTGTTAAAAATATATATTTGTGGCGGGCTTACCTACATCAAATTAATAAGAGACTAATTATAAACTAAAC |
| | | ACACAAGCAAGCTACTTTAGGGTAAAAGTTTATAAATGCTTTTGACGTATAAACGTTGCTTGTATTTATTATTACAATTAAAGG |
| | | TGGATAGAAAACCTAGAGACTAGTTAGAAACTAATCTCAGGTTTGCGTTAAACTAAATCAGAGCCCGAGAGGTTAACAGAAC |
| | | CTAGAAGGGGACTAGATATCCGGGTAGGGAAACAAAAAAAAAAAACAAGACAGCCACATATTAGGGAGACTAGTTAGAAGC |
| | | TAGTTCCAGGACTAGGAAAATAAAAGACAATGATACCACAGTCTAGTTGACAACTAGATAGATTCTAGATTGAGGCCAAAGT |
| | | CTCTGAGATCCAGGTTAGTTGCAACTAATACTAGTTAGTATCTAGTCTCCTATAACTCTGAAGCTAGAATAACTTACTACTATT |
| | | ATCCTCACCACTGTTCAGCTGCGCAAACGGAGTGATTGCAAGGTGTTCAGAGACTAGTTATTGACTAGTCAGTGACTAGCAA |
| | | TAACTAACAAGGTATTAACCTACCATGTCTGCCATCACCCTGCACTTCCTCGGGCTCAGCAGCCTTTTCCTCCTCATTTTCAT |
| | | GCTCATTTTCCTTGTTTAAGACTGTGACTAGTCAAAGACTAGTCCAGAACCACAAAGGAGAAATGTCTTACCACTTTCTTCAT |
| | | TGCTTGTCTCTTTTGCATTATCCATGTCTGCAACTAGTTAGAGTCTAGTTAGTGACTAGTCCGACGAGGACTTGCTTGTCTCC |
| | | GGATTGTTGGAGGAACTCTCCAGGGCCTCAAGATCCACAACAGAGCCTTCTAGAAGACTGGTCAATAACTAGTTGGTCTTTG |
| | | TCTGAGTCTGACTTACGAGGTTGCATACTCGCTCCCTTTGCCTCGTCAATCGATGAGAAAAAGCGCCAAAACTCGCAATATG |
| | | GCTTTGAACCACACGGTGCTGAGACTAGTTAGAATCTAGTCCCAAACTAGCTTGGATAGCTTACCTTTGCCCTTTGCGTTGC |
| | | GACAGGTCTTGCAGGGTATGGTTCCTTTCTCACCAGCTGATTTAGCTGCCTTGCTACCCTCACGGCGGATCTGCATAAAGA |
| | | GTGGCTAGAGGTTATAAATTAGCACTGATCCTAGGTACGGGGCTGAATGTAACTTGCCTTTCCTTTCTCATCGCGCGGCAAG |
| | | ACAGGCTTGCTCAAATTCCTACCAGTCACAGGGGTATGCACGGCGTACGGACCACTTGAACTAGTCACAGATTAGTTAGCA |
| | | ACTAGTCTGCATTGAATGGCTGTACTTACGGGCCCTCGCCATTGTCCTGATCATTTCCAGCTTCACCCTCGTTGCTGCAAAG |
| | | TAGTTAGTGACTAGTCAAGGACTAGTTGAAATGGGAGAAGAAACTCACGAATTCTCGACACCCTTAGTATTGTGGTCCTTGG |
| | | ACTTGGTGCTGCTATATATTAGCTAATACACTAGTTAGACTCACAGAAACTTACGCAGCTCGCTTGCGCTTCTTGGTAGGAG |
| | | TCGGGGTTGGGAGAACAGTGCCTTCAAACAAGCCTTCATACCATGCTACTTGACTAGTCAGGGACTAGTCACCAAGTAATCT |
| | | AGATAGGACTTGCCTTTGGCCTCCATCAGTTCCTTCATAGTGGGAGGTCCATTGTGCAATGTAAACTCCATGCCGTGGGAGT |
| | | TCTTGTCCTTCAAGTGCTTGACCAATATGTTTCTGTTGGCAGAGGGAACCTGTCAACTAGTTAATAACTAGTCAGAAACTAGT |
| | | ATAGCAGTAGACTCACTGTACGCTTGAGGCATCCCTTCACTCGGCAGTAGACTTCATATGGATGGATATCAGGCACGCCATT |
| | | GTCGTCCTGTGGACTAGTCAGTAACTAGGCTTAAAGCTAGTCGGGTCGGCTTACTATCTTGAAATCCGGCAGCGTAAGCTC |
| | | CCCGTCCTTAACTGCCTCGAGATAGTGACAGTACTCTGGGGACTTTCGGAGATCGTTATCGCGAATGCTCGGCATACTAAT |
| | | CGTTGACTAGTCTTGGACTAGTCCCGAGCAAAAAGGATTGGAGGAGGAGGAGGAAGGTGAGAGTGAGACAAAGAGCGAAA |
| | | TAAGAGCTTCAAAGGCTATCTCTAAGCAGTATGAAGGTTAAGTATCTAGTTCTTGACTAGATTTAAAAGAGATTTCGACTAGT |
| | | TATGTACCTGGAGTTTGGATATAGGAATGTGTTGTGGTAACGAAATGTAAGGGGGAGGAAAGAAAAAGTCGGTCAAGAGGT |
| | | AACTCTAAGTCGGCCATTCCTTTTTGGGAGGCGCTAACCATAAACGGCATGGTCGACTTAGAGTTAGCTCAGGGAATTTAGG |
| | | GAGTTATCTGCGACCACCGAGGAACGGCGGAATGCCAAAGAATCCCGATGGAGCTCTAGCTGGCGGTTGACAACCCCACC |
| | | TTTTGGCGTTTCTGCGGCGTTGCAGGCGGGACTGGATACTTCGTAGAACCAGAAAGGCAAGGCAGAACGCGCTCAGCAAG |
| | | AGTGTTGGAAGTGATAGCATGATGTGCCTTGTTAACTAGGTCAAAATCTGCAGTATGCTTGATGTTATCCAAAGTGTGAGAG |
| | | AGGAAGGTCCAAACATACACGATTGGGAGAGGGCCTAGGTATAAGAGTTTTTGAGTAGAACGCATGTGAGCCCAGCCATCT |
| | | CGAGGAGATTAAACACGGGCCGGCATTTGATGGCTATGTTAGTACCCCAATGGAAAGCCTGAGAGTCCAGTGGTCGCAGAT |
| | | AACTCCCTAAATTCCCTGAGCTAACTCTAAGTCGACCATGCCGTTTATGGTTAGCGCCTCCCAAAAAGGAATGGCCGACTTA |
| | | GAGTTACCTCTTGACCGACTTTTTCTTTCCTCCCCCTTACATTTCGTTACCACAACACATTCCTATATCCAAACTCCAGGTACA |
| | | TAACTAGTCGAAATCTCTTTTAAATCTAGTCAAGAACTAGATACTTAACCTTCATACTGCTTAGAGATAGCCTTTGAAGCTCTT |
| | | ATTTCGCTCTTTGTCTCACTCTCACCTTCCTCCTCCTCCTCCAATCCTTTTTGCTCGGGACTAGTCCAAGACTAGTCAACGAT |
| | | TAGTATGCCGAGCATTCGCGATAACGATCTCCGAAAGTCCCCAGAGTACTGTCACTATCTCGAGGCAGTTAAGGACGGGGA |
| | | GCTTACGCTGCCGGATTTCAAGATAGTAAGCCGACCCGACTAGCTTTAAGCCTAGTTACTGACTAGTCCACAGGACGACAAT |
| | | GGCGTGCCTGATATCCATCCATATGAAGTCTACTGCCGAGTGAAGGGATGCCTCAAGCGTACAGTGAGTCTACTGCTATAC |
| | | TAGTTTCTGACTAGTTATTAACTAGTTGACAGGTTCCCTCTGCCAACAGAAACATATTGGTCAAGCACTTGAAGGACAAGAAC |
| | | TCCCACGGCATGGAGTTTACATTGCACAATGGACCTCCCACTATGAAGGAACTGATGGAGGCCAAAGGCAAGTCCTATCTA |
| | | GATTACTTGGTGACTAGTCCCTGACTAGTCAAGTAGCATGGTATGAAGGCTTGTTTGAAGGCACTGTTCTCCCAACCCCGAC |
| | | TCCTACCAAGAAGCGCAAGCGAGCTGCGTAAGTTTCTGTGAGTCTAACTAGTGTATTAGCTAATATATAGCAGCACCAAGTC |
| | | CAAGGACCACAATACTAAGGGTGTCGAGAATTCGTGAGTTTCTTCTCCCATTTCAACTAGTCCTTGACTAGTCACTAACTACT |
| | | TTGCAGCAACGAGGGTGAAGCTGGAAATGATCAGGACAATGGCGAGGGCCCGTAAGTACAGCCATTCAATGCAGACTAGTT |
| | | GCTAACTAATCTGTGACTAGTTCAAGTGGTCCGTACGCCGTGCATACCCCTGTGACTGGTAGGAATTTGAGCAAGCCTGTCT |
| | | TGCCGCGCGATGAGAAAGGAAAGGCAAGTTACATTCAGCCCCGTACCTAGGATCAGTGCTAATTTATAACCTCTAGCCACT |
| | | CTTTATGCAGATCCGCCGTGAGGGTAGCAAGGCAGCTAAATCAGCTGGTGAGAAAGGAACCATACCCTGCAAGACCTGTCG |
| | | CAACGCAAAGGGCAAAGGTAAGCTATCCAAGCTAGTTTGGGACTAGATTCTAACTAGTCTCAGCACCGTGTGGTTCAAAGC |
| | | CATATTGCGAGTTTTGGCGCTTTTTCTCATCGATTGACGAGGCAAAGGGAGCGAGTATGCAACCTCGTAAGTCAGACTCAGA |
| | | CAAAGACCAACTAGTTATTGACCAGTCTTCTAGAAGGCTCTGTTGTGGATCTTGAGGCCCTGGAGAGTTCCTCCAACAATCC |
| | | GGAGACAAGCAAGTCCTCGTCGGACTAGTCACTAACTAGACTCTAACTAGTTGCAGACATGGATAATGCAAAAGAGACAAG |
| | | CAATGAAGAAAGTGGTAAGACATTTCTCCTTTGTGGTTCTGGACTAGTCTTTGACTAGTCACAGTCTTAAACAAGGAAAATGA |
| | | GCATGAAAATGAGGAGGAAAAGGCTGCTGAGCCCGAGGAAGTGCAGGGTGATGGCAGACATGGTAGGTTAATACCTTGTT |
| | | AGTTATTGCTAGTCACTGACTAGTCAATAACTAGTCTCTGAACACCTTGCAATCACTCCGTTTGCGCAGCTGAACAGTGGTG |
| | | AGGATAATAGTAGTAAGTTATTCTAGCTTCAGAGTTATAGGAGACTAGATACTAACTAGTATTAGTTGCAACTAACCTGGATC |
| | | TCAGAGACTTTGGCCTCAATCTAGAATCTATCTAGTTGTCAACTAGACTGTGGTATCATTGTCTTTTATTTTCCTAGTCCTGGA |
| | | ACTAGCTTCTAACTAGTCTCCCTAATATGTGGCTGTCTTGTTTTTTTTTTTTGTTTCCCTACCCGGATATCTAGTCCCCTTCTA |
| | | GGTTCTGTTAACCTCTCGGGCTCTGATTTAGTTTAACGCAAACCTGAGATTAGTTTCTAACTAGTCTCTAGGTTTTCTATCCA |
| | | CCTTTAATTGTAATAATAAATACAAGCAACGTTTATACGTCAAAAGCATTTATAAACTTTTACCCTAAAGTAGCTTGCTTGTGT |
| | | GTTTAGTTTATAATTAGTCTCTTATTAATTTGATGTAGGTAAGCCCGCCACAAATATATATTTTTAACAAGATACCGTGGAAAA |
| | | ACTTCGTGCTATCACAAAACAGTATACAAAAAATAAGCTTGGTACCCATGACACATGCATCCACCATCGCAGACTTATCATCA |
| | | CTGAGCCTCCACCTAGCCTGCGGCCGCCTTGACGACCGTTGATCTGCTTGATCTCGTCTCCCGAAAATGAAAATAGCTCTG |
| | | CTAAGCTATTCTTCTCTTCGCCGGAGCCTGAAGGCGTTACTAGGTTGCAGTCAATGCATTAATGCATTGCAGATGAGCTGTA |
| | | TCTGGAAGAGGTAAACCCGAAAACGCGTTTTATTCTTGTTGACATGGAGCTATTAAATCACTAGAAGGCACTCTTTGCTGCTT |
| | | GGACAAATGAACGTATCTTATCGAGATCCTGAACACCATTTGTCTCAACTCCGGAGCTGACATCGACACCAACGATCTTATA |
| | | TCCAGATTCGTCAAGCTGTTTGATGATTTCAGTAACGTTAAGTGGATCCCTATTCCTTTGCCCTCGGACGAGTGCTGGGGCG |
| | | TCGGTTTCCACTATCGGCGAGTACTTCTACACAGCCATCGGTCCAGACGGCCGCGCTTCTGCGGGCGATTTGTGTACGCCC |
| | | GACAGTCCCGGCTCCGGATCGGACGATTGCGTCGCATCGACCCTGCGCCCAAGCTGCATCATCGAAATTGCCGTCAACCA |
| | | AGCTCTGATAGAGTTGGTCAAGACCAATGCGGAGCATATACGCCCGGAGCCGCGGCGATCCTGCAAGCTCCGGATGCCTC |
| | | CGCTCGAAGTAGCGCGTCTGCTGCTCCATACAAGCCAACCACGGCCTCCAGAAGAAGATGTTGGCGACCTCGTATTGGGA |
| | | ATCCCCGAACATCGCCTCGCTCCAGTCAATGACCGCTGTTATGCGGCCATTGTCCGTCAGGACATTGTTGGAGCCGAAATC |
| | | CGCGTGCACGAGGTGCCGGACTTCGGGGCAGTCCTCGGCCCAAAGCATCAGCTCATCGAGAGCCTGCGCGACGGACGCA |
| | | CTGACGGTGTCGTCCATCACAGTTTGCCAGTGATACACATGGGGATCAGCAATCGCGCATATGAAATCACGCCATGTAGTG |
| | | TATTGACCGATTCCTTGCGGTCCGAATGGGCCGAACCCGCTCGTCTGGCTAAGATCGGCCGCAGCGATCGCATCCATGGC |
| | | CTCCGCGACCGGCTGCAGAACAGCGGGCAGTTCGGTTTCAGGCAGGTCTTGCAACGTGACACCCTGTGCACGGCGGGAG |
| | | ATGCAATAGGTCAGGCTCTCGCTGAACTCCCCAATGTCAAGCACTTCCGGAATCGGGAGCGCGGCCGATGCAAAGTGCCG |
| | | ATAAACATAACGATCTTTGTAGAAACCATCGGCGCAGCTATTTACCCGCAGGACATATCCACGCCCTCCTACATCGAAGCTG |
| | | AAAGCACGAGATTCTTCGCCCTCCGAGAGCTGCATCAGGTCGGAGACGCTGTCGAACTTTTCGATCAGAAACTTCTCGACA |
| | | GACGTCGCGGTGAGTTCAGGCTTTTTCATATCGATTGTGATGTGATGGAGTTGAGATGGGGGTGAGGAGATGGATGATGGG |
| | | AAAGGAAGATGGACTGAGGATGGAAGAAGAGAAGAAGAGAGAGAGAGAAAGTCTTCCAGGAGAGAAAGGGAACCGAAGAA |
| | | AAATGGGGAGGAAACCGGCCCTAGCACCTAAATACGTCTCCCGCTTAATTTTCGCCCTTTTTTCACCAAACCCTCCGCGTCT |
| | | TTCGTGCGCTAGCTGTCTTGGGGGGTGTGTAAAACTTGGGAACAACCCTACGCCGAACCTCCCGTACGAAGCCCGTACAGT |
| | | GTATTCTATCCCTGGCTTTCCCAGCCTGGTAAGTCGGGTCCCTTCGGGACGGGGCCAAGGAGACTGAGTTTCCGGGTTAAC |
| | | CAATAATGCCGCCAGCCGTGGAGCGGTCTGAGCTGTCTATCGTGAATCCGTGACGCTGAATTGCTCAGTCCAAGTCGGAGA |
| | | CGCTGGAATCCACCGGTTGCTCCAGCCACGGCGAAGAATCCACTTACCTTCCGGGCTTCCGCCAGCTGGCAACATATTTTT |
| | | GAGGCTGATATAGCCTTCTCCTCCATCACGATAAGCCCTGACTACCTTGCTACGGGCCAATTGCAATTTGTTTGCTGGTTAC |
| | | GCTTTACAAAAGGTGGCCGTTACTGAGCAAAAAGAAAATGATGTAAAAATTTGCGAGTGGGTCCCATAGCTGGATGGGTCC |
| | | GATAAAATGGTACTGCCCCACTTAGTGGCAGCTCGCGACCAGTCACAAGCCCAGGACCGCAAGCTAGAATGATAGACGGC |
| | | ACCTCGGACGCAAATCTTGACCAATCCGGCCCGAATCGGGAATGCGGCTCCACAGCTGCAGGTTTAAACTAGCAGAAGAG |
| | | CCTGGGCCTCATGGGCCTTCCGCTCACTGCCCGCTTTCCAGTCGGGAAACCTGTCGTGCCAGCTGCATTAACATGGTCATA |
| | | GCTGTTTCCTTGCGTATTGGGCGCTCTCCGCTTCCTCGCTCACTGACTCGCTGCGCTCGGTCGTTCGGGTAAAGCCTGGG |
| | | GTGCCTAATGAGCAAAAGGCCAGCAAAAGGCCAGGAACCGTAAAAAGGCCGCGTTGCTGGCGTTTTTCCATAGGCTCCGC |
| | | CCCCCTGACGAGCATCACAAAAATCGACGCTCAAGTCAGAGGTGGCGAAACCCGACAGGACTATAAAGATACCAGGCGTTT |
| | | CCCCCTGGAAGCTCCCTCGTGCGCTCTCCTGTTCCGACCCTGCCGCTTACCGGATACCTGTCCGCCTTTCTCCCTTCGGGA |
| | | AGCGTGGCGCTTTCTCATAGCTCACGCTGTAGGTATCTCAGTTCGGTGTAGGTCGTTCGCTCCAAGCTGGGCTGTGTGCAC |
| | | GAACCCCCCGTTCAGCCCGACCGCTGCGCCTTATCCGGTAACTATCGTCTTGAGTCCAACCCGGTAAGACACGACTTATCG |
| | | CCACTGGCAGCAGCCACTGGTAACAGGATTAGCAGAGCGAGGTATGTAGGCGGTGCTACAGAGTTCTTGAAGTGGTGGCC |
| | | TAACTACGGCTACACTAGAAGAACAGTATTTGGTATCTGCGCTCTGCTGAAGCCAGTTACCTTCGGAAAAAGAGTTGGTAGC |
| | | TCTTGATCCGGCAAACAAACCACCGCTGGTAGCGGTGGTTTTTTTGTTTGCAAGCAGCAGATTACGCGCAGAAAAAAAGGAT |
| | | CTCAAGAAGATCCTTTGATCTTTTCTACGGGGTCTGACGCTCAGTGGAACGAAAACTCACGTTAAGGGATTTTGGTCATGAG |
| | | ATTATCAAAAAGGATCTTCACCTAGATCCTTTTAAATTAAAAATGAAGTTTTAAATCAATCTAAAGTATATATGAGTAAACTTGG |
| | | TCTGACAGTTACCAATGCTTAATCAGTGAGGCACCTATCTCAGCGATCTGTCTATTTCGTTCATCCATAGTTGCCTGACTCCC |
| | | CGTCGTGTAGATAACTACGATACGGGAGGGCTTACCATCTGGCCCCAGTGCTGCAATGATACCGCGAGAACCACGCTCAC |
| | | CGGCTCCAGATTTATCAGCAATAAACCAGCCAGCCGGAAGGGCCGAGCGCAGAAGTGGTCCTGCAACTTTATCCGCCTCC |
| | | ATCCAGTCTATTAATTGTTGCCGGGAAGCTAGAGTAAGTAGTTCGCCAGTTAATAGTTTGCGCAACGTTGTTGCCATTGCTA |
| | | CAGGCATCGTGGTGTCACGCTCGTCGTTTGGTATGGCTTCATTCAGCTCCGGTTCCCAACGATCAAGGCGAGTTACATGAT |
| | | CCCCCATGTTGTGCAAAAAAGCGGTTAGCTCCTTCGGTCCTCCGATCGTTGTCAGAAGTAAGTTGGCCGCAGTGTTATCACT |
| | | CATGGTTATGGCAGCACTGCATAATTCTCTTACTGTCATGCCATCCGTAAGATGCTTTTCTGTGACTGGTGAGTACTCAACCA |
| | | AGTCATTCTGAGAATAGTGTATGCGGCGACCGAGTTGCTCTTGCCCGGCGTCAATACGGGATAATACCGCGCCACATAGCA |
| | | GAACTTTAAAAGTGCTCATCATTGGAAAACGTTCTTCGGGGCGAAAACTCTCAAGGATCTTACCGCTGTTGAGATCCAGTTC |
| | | GATGTAACCCACTCGTGCACCCAACTGATCTTCAGCATCTTTTACTTTCACCAGCGTTTCTGGGTGAGCAAAAACAGGAAGG |
| | | CAAAATGCCGCAAAAAAGGGAATAAGGGCGACACGGAAATGTTGAATACTCATACTCTTCCTTTTTCAATATTATTGAAGCAT |
| | | TTATCAGGGTTATTGTCTCATGAGCGGATACATATTTGAATGTATTTAGAAAAATAAACAAATAGGGGTTCCGCGCACATTTC |
| | | CCCGAAAAGTGCCAC |
| SEQ | 17 | CTAAATTGTAAGCGTTAATATTTTGTTAAAATTCGCGTTAAATTTTTGTTAAATCAGCTCATTTTTTAACCAATAGGCCGAAATC |
| ID | | GGCAAAATCCCTTATAAATCAAAAGAATAGACCGAGATAGGGTTGAGTGGCCGCTACAGGGCGCTCCCATTCGCCATTCAG |
| NO | | GCTGCGCAACTGTTGGGAAGGGCGTTTCGGTGCGGGCCTCTTCGCTATTACGCCAGCTGGCGAAAGGGGGATGTGCTGCA |
| | | AGGCGATTAAGTTGGGTAACGCCAGGGTTTTCCCAGTCACGACGTTGTAAAACGACGGCCAGTGAGCGCGACGTAATACG |
| | | ACTCACTATAGGGCGAATTGGCGGAAGGCCGTCAAGGCCGCATAGCTTATTTTTTGTATACTGTTTTGTGATAGCACGAAGT |
| | | TTTTCCACGGTATCTTGTTAAAAATATATATTTGTGGCGGGCTTACCTACATCAAATTAATAAGAGACTAATTATAAACTAAAC |
| | | ACACAAGCAAGCTACTTTAGGGTAAAAGTTTATAAATGCTTTTGACGTATAAACGTTGCTTGTATTTATTATTACAATTAAAGG |
| | | TGGATAGAAAACCTAGAGACTAGTTAGAAACTAATCTCAGGTTTGCGTTAAACTAAATCAGAGCCCGAGAGGTTAACAGAAC |
| | | CTAGAAGGGGACTAGATATCCGGGTAGGGAAACAAAAAAAAAAAACAAGACAGCCACATATTAGGGAGACTAGTTAGAAGC |
| | | TAGTTCCAGGACTAGGAAAATAAAAGACAATGATACCACAGTCTAGTTGACAACTAGATAGATTCTAGATTGAGGCCAAAGT |
| | | CTCTGAGATCCAGGTTAGTTGCAACTAATACTAGTTAGTATCTAGTCTCCTATAACTCTGAAGCTAGAATAACTTACTACTATT |
| | | ATCCTCACCACTGTTCAGCTGCGCAAACGGAGTGATTGCAAGGTGTTCAGAGACTAGTTATTGACTAGTCAGTGACTAGCAA |
| | | TAACTAACAAGGTATTAACCTACCATGTCTGCCATCACCCTGCACTTCCTCGGGCTCAGCAGCCTTTTCCTCCTCATTTTCAT |
| | | GCTCATTTTCCTTGTTTAAGACTGTGACTAGTCAAAGACTAGTCCAGAACCACAAAGGAGAAATGTCTTACCACTTTCTTCAT |
| | | TGCTTGTCTCTTTTGCATTATCCATGTCTGCAACTAGTTAGAGTCTAGTTAGTGACTAGTCCGACGAGGACTTGCTTGTCTCC |
| | | GGATTGTTGGAGGAACTCTCCAGGGCCTCAAGATCCACAACAGAGCCTTCTAGAAGACTGGTCAATAACTAGTTGGTCTTTG |
| | | TCTGAGTCTGACTTACGAGGTTGCATACTCGCTCCCTTTGCCTCGTCAATCGATGAGAAAAAGCGCCAAAACTCGCAATATG |
| | | GCTTTGAACCACACGGTGCTGAGACTAGTTAGAATCTAGTCCCAAACTAGCTTGGATAGCTTACCTTTGCCCTTTGCGTTGC |
| | | GACAGGTCTTGCAGGGTATGGTTCCTTTCTCACCAGCTGATTTAGCTGCCTTGCTACCCTCACGGCGGATCTGCATAAAGA |
| | | GTGGCTAGAGGTTATAAATTAGCACTGATCCTAGGTACGGGGCTGAATGTAACTTGCCTTTCCTTTCTCATCGCGCGGCAAG |
| | | ACAGGCTTGCTCAAATTCCTACCAGTCACAGGGGTATGCACGGCGTACGGACCACTTGAACTAGTCACAGATTAGTTAGCA |
| | | ACTAGTCTGCATTGAATGGCTGTACTTACGGGCCCTCGCCATTGTCCTGATCATTTCCAGCTTCACCCTCGTTGCTGCAAAG |
| | | TAGTTAGTGACTAGTCAAGGACTAGTTGAAATGGGAGAAGAAACTCACGAATTCTCGACACCCTTAGTATTGTGGTCCTTGG |
| | | ACTTGGTGCTGCTATATATTAGCTAATACACTAGTTAGACTCACAGAAACTTACGCAGCTCGCTTGCGCTTCTTGGTAGGAG |
| | | TCGGGGTTGGGAGAACAGTGCCTTCAAACAAGCCTTCATACCATGCTACTTGACTAGTCAGGGACTAGTCACCAAGTAATCT |
| | | AGATAGGACTTGCCTTTGGCCTCCATCAGTTCCTTCATAGTGGGAGGTCCATTGTGCAATGTAAACTCCATGCCGTGGGAGT |
| | | TCTTGTCCTTCAAGTGCTTGACCAATATGTTTCTGTTGGCAGAGGGAACCTGTCAACTAGTTAATAACTAGTCAGAAACTAGT |
| | | ATAGCAGTAGACTCACTGTACGCTTGAGGCATCCCTTCACTCGGCAGTAGACTTCATATGGATGGATATCAGGCACGCCATT |
| | | GTCGTCCTGTGGACTAGTCAGTAACTAGGCTTAAAGCTAGTCGGGTCGGCTTACTATCTTGAAATCCGGCAGCGTAAGCTC |
| | | CCCGTCCTTAACTGCCTCGAGATAGTGACAGTACTCTGGGGACTTTCGGAGATCGTTATCGCGAATGCTCGGCATACTAAT |
| | | CGTTGACTAGTCTTGGACTAGTCCCGAGCAAAAAGGATTGGAGGAGGAGGAGGAAGGTGAGAGTGAGACAAAGAGCGAAA |
| | | TAAGAGCTTCAAAGGCTATCTCTAAGCAGTATGAAGGTTAAGTATCTAGTTCTTGACTAGATTTAAAAGAGATTTCGACTAGT |
| | | TATGTACCTGGAGTTTGGATATAGGAATGTGTTGTGGTAACGAAATGTAAGGGGGAGGAAAGAAAAAGTCGGTCAAGAGGT |
| | | AACTCTAAGTCGGCCATTCCTTTTTGGGAGGCGCTAACCATAAACGGCATGGTCGACTTAGAGTTAGCTCAGGGAATTTAGG |
| | | GAGTTATCTGCGACCACCGAGGAACGGCGGAATGCCAAAGAATCCCGATGGAGCTCTAGCTGGCGGTTGACAACCCCACC |
| | | TTTTGGCGTTTCTGCGGCGTTGCAGGCGGGACTGGATACTTCGTAGAACCAGAAAGGCAAGGCAGAACGCGCTCAGCAAG |
| | | AGTGTTGGAAGTGATAGCATGATGTGCCTTGTTAACTAGGTCAAAATCTGCAGTATGCTTGATGTTATCCAAAGTGTGAGAG |
| | | AGGAAGGTCCAAACATACACGATTGGGAGAGGGCCTAGGTATAAGAGTTTTTGAGTAGAACGCATGTGAGCCCAGCCATCT |
| | | CGAGGAGATTAAACACGGGCCGGCATTTGATGGCTATGTTAGTACCCCAATGGAAAGCCTGAGAGTCCAGTGGTCGCAGAT |
| | | AACTCCCTAAATTCCCTGAGCTAACTCTAAGTCGACCATGCCGTTTATGGTTAGCGCCTCCCAAAAAGGAATGGCCGACTTA |
| | | GAGTTACCTCTTGACCGACTTTTTCTTTCCTCCCCCTTACATTTCGTTACCACAACACATTCCTATATCCAAACTCCAGGTACA |
| | | TAACTAGTCGAAATCTCTTTTAAATCTAGTCAAGAACTAGATACTTAACCTTCATACTGCTTAGAGATAGCCTTTGAAGCTCTT |
| | | ATTTCGCTCTTTGTCTCACTCTCACCTTCCTCCTCCTCCTCCAATCCTTTTTGCTCGGGACTAGTCCAAGACTAGTCAACGAT |
| | | TAGTATGCCGAGCATTCGCGATAACGATCTCCGAAAGTCCCCAGAGTACTGTCACTATCTCGAGGCAGTTAAGGACGGGGA |
| | | GCTTACGCTGCCGGATTTCAAGATAGTAAGCCGACCCGACTAGCTTTAAGCCTAGTTACTGACTAGTCCACAGGACGACAAT |
| | | GGCGTGCCTGATATCCATCCATATGAAGTCTACTGCCGAGTGAAGGGATGCCTCAAGCGTACAGTGAGTCTACTGCTATAC |
| | | TAGTTTCTGACTAGTTATTAACTAGTTGACAGGTTCCCTCTGCCAACAGAAACATATTGGTCAAGCACTTGAAGGACAAGAAC |
| | | TCCCACGGCATGGAGTTTACATTGCACAATGGACCTCCCACTATGAAGGAACTGATGGAGGCCAAAGGCAAGTCCTATCTA |
| | | GATTACTTGGTGACTAGTCCCTGACTAGTCAAGTAGCATGGTATGAAGGCTTGTTTGAAGGCACTGTTCTCCCAACCCCGAC |
| | | TCCTACCAAGAAGCGCAAGCGAGCTGCGTAAGTTTCTGTGAGTCTAACTAGTGTATTAGCTAATATATAGCAGCACCAAGTC |
| | | CAAGGACCACAATACTAAGGGTGTCGAGAATTCGTGAGTTTCTTCTCCCATTTCAACTAGTCCTTGACTAGTCACTAACTACT |
| | | TTGCAGCAACGAGGGTGAAGCTGGAAATGATCAGGACAATGGCGAGGGCCCGTAAGTACAGCCATTCAATGCAGACTAGTT |
| | | GCTAACTAATCTGTGACTAGTTCAAGTGGTCCGTACGCCGTGCATACCCCTGTGACTGGTAGGAATTTGAGCAAGCCTGTCT |
| | | TGCCGCGCGATGAGAAAGGAAAGGCAAGTTACATTCAGCCCCGTACCTAGGATCAGTGCTAATTTATAACCTCTAGCCACT |
| | | CTTTATGCAGATCCGCCGTGAGGGTAGCAAGGCAGCTAAATCAGCTGGTGAGAAAGGAACCATACCCTGCAAGACCTGTCG |
| | | CAACGCAAAGGGCAAAGGTAAGCTATCCAAGCTAGTTTGGGACTAGATTCTAACTAGTCTCAGCACCGTGTGGTTCAAAGC |
| | | CATATTGCGAGTTTTGGCGCTTTTTCTCATCGATTGACGAGGCAAAGGGAGCGAGTATGCAACCTCGTAAGTCAGACTCAGA |
| | | CAAAGACCAACTAGTTATTGACCAGTCTTCTAGAAGGCTCTGTTGTGGATCTTGAGGCCCTGGAGAGTTCCTCCAACAATCC |
| | | GGAGACAAGCAAGTCCTCGTCGGACTAGTCACTAACTAGACTCTAACTAGTTGCAGACATGGATAATGCAAAAGAGACAAG |
| | | CAATGAAGAAAGTGGTAAGACATTTCTCCTTTGTGGTTCTGGACTAGTCTTTGACTAGTCACAGTCTTAAACAAGGAAAATGA |
| | | GCATGAAAATGAGGAGGAAAAGGCTGCTGAGCCCGAGGAAGTGCAGGGTGATGGCAGACATGGTAGGTTAATACCTTGTT |
| | | AGTTATTGCTAGTCACTGACTAGTCAATAACTAGTCTCTGAACACCTTGCAATCACTCCGTTTGCGCAGCTGAACAGTGGTG |
| | | AGGATAATAGTAGTAAGTTATTCTAGCTTCAGAGTTATAGGAGACTAGATACTAACTAGTATTAGTTGCAACTAACCTGGATC |
| | | TCAGAGACTTTGGCCTCAATCTAGAATCTATCTAGTTGTCAACTAGACTGTGGTATCATTGTCTTTTATTTTCCTAGTCCTGGA |
| | | ACTAGCTTCTAACTAGTCTCCCTAATATGTGGCTGTCTTGTTTTTTTTTTTTGTTTCCCTACCCGGATATCTAGTCCCCTTCTA |
| | | GGTTCTGTTAACCTCTCGGGCTCTGATTTAGTTTAACGCAAACCTGAGATTAGTTTCTAACTAGTCTCTAGGTTTTCTATCCA |
| | | CCTTTAATTGTAATAATAAATACAAGCAACGTTTATACGTCAAAAGCATTTATAAACTTTTACCCTAAAGTAGCTTGCTTGTGT |
| | | GTTTAGTTTATAATTAGTCTCTTATTAATTTGATGTAGGTAAGCCCGCCACAAATATATATTTTTAACAAGATACCGTGGAAAA |
| | | ACTTCGTGCTATCACAAAACAGTATACAAAAAATAAGCTTGGTACCTCATGACACATGCATCCACCATCGCAGACTTATCATC |
| | | ACTGAGCCTCCACCTAGCCTGCGGCCGCCTTGACGACCGTTGATCTGCTTGATCTCGTCTCCCGAAAATGAAAATAGCTCT |
| | | GCTAAGCTATTCTTCTCTTCGCCGGAGCCTGAAGGCGTTACTAGGTTGCAGTCAATGCATTAATGCATTGCAGATGAGCTGT |
| | | ATCTGGAAGAGGTAAACCCGAAAACGCGTTTTATTCTTGTTGACATGGAGCTATTAAATCACTAGAAGGCACTCTTTGCTGCT |
| | | TGGAC4AAATGAACGTATCTTATCGAGATCCTGAACACCATTTGTCTCAACTCCGGAGCTGACATCGACACCAACGATCTTAT |
| | | ATCCAGATTCGTCAAGCTGTTTGATGATTTCAGTAACGTTAAGTGGATCCTTTAGTCCTGCTCCTCGGCCACGAAGTGCACG |
| | | CAGTTGCCGGCCGGGTCGCGCAGGGCGAACTCCCGCCCCCACGGCTGCTCGCCGATCTCGGTCATGGCCGGCCCGGAG |
| | | GCGTCCCGGAAGTTCGTGGACACGACCTCCGACCACTCGGCGTACAGCTCGTCCAGGCCGCGCACCCACACCCAGGCCA |
| | | GGGTGTTGTCCGGCACCACCTGGTCCTGGACCGCGCTGATGAACAGGGTCACGTCGTCCCGGACCACACCGGCGAAGTC |
| | | GTCCTCCACGAAGTCCCGGGAGAACCCGAGCCGGTCGGTCCAGAACTCGACCGCTCCGGCGACGTCGCGCGCGGTGAGC |
| | | ACCGGAACGGCACTGGTCAACTTGGCCATATCGATTGTGATGTGATGGAGTTGAGATGGGGGTGAGGAGATGGATGATGG |
| | | GAAAGGAAGATGGACTGAGGATGGAAGAAGAGAAGAAGAGAGAGAGAGAAAGTCTTCCAGGAGAGAAAGGGAACCGAAGA |
| | | AAAATGGGGAGGAAACCGGCCCTAGCACCTAAATACGTCTCCCGCTTAATTTTCGCCCTTTTTTCACCAAACCCTCCGCGTC |
| | | TTTCGTGCGCTAGCTGTCTTGGGGGGTGTGTAAAACTTGGGAACAACCCTACGCCGAACCTCCCGTACGAAGCCCGTACAG |
| | | TGTATTCTATCCCTGGCTTTCCCAGCCTGGTAAGTCGGGTCCCTTCGGGACGGGGCCAAGGAGACTGAGTTTCCGGGTTAA |
| | | CCAATAATGCCGCCAGCCGTGGAGCGGTCTGAGCTGTCTATCGTGAATCCGTGACGCTGAATTGCTCAGTCCAAGTCGGA |
| | | GACGCTGGAATCCACCGGTTGCTCCAGCCACGGCGAAGAATCCACTTACCTTCCGGGCTTCCGCCAGCTGGCAACATATTT |
| | | TTGAGGCTGATATAGCCTTCTCCTCCATCACGATAAGCCCTGACTACCTTGCTACGGGCCAATTGCAATTTGTTTGCTGGTT |
| | | ACGCTTTACAAAAGGTGGCCGTTACTGAGCAAAAAGAAAATGATGTAAAAATTTGCGAGTGGGTCCCATAGCTGGATGGGT |
| | | CCGATAAAATGGTACTGCCCCACTTAGTGGCAGCTCGCGACCAGTCACAAGCCCAGGACCGCAAGCTAGAATGATAGACG |
| | | GCACCTCGGACGCAAATCTTGACCAATCCGGCCCGAATCGGGAATGCGGCTCCACAGCTGCAGGTTTAAACTAGCAGAAG |
| | | AGCCTGGGCCTCATGGGCCTTCCGCTCACTGCCCGCTTTCCAGTCGGGAAACCTGTCGTGCCAGCTGCATTAACATGGTCA |
| | | TAGCTGTTTCCTTGCGTATTGGGCGCTCTCCGCTTCCTCGCTCACTGACTCGCTGCGCTCGGTCGTTCGGGTAAAGCCTGG |
| | | GGTGCCTAATGAGCAAAAGGCCAGCAAAAGGCCAGGAACCGTAAAAAGGCCGCGTTGCTGGCGTTTTTCCATAGGCTCCG |
| | | CCCCCCTGACGAGCATCACAAAAATCGACGCTCAAGTCAGAGGTGGCGAAACCCGACAGGACTATAAAGATACCAGGCGTT |
| | | TCCCCCTGGAAGCTCCCTCGTGCGCTCTCCTGTTCCGACCCTGCCGCTTACCGGATACCTGTCCGCCTTTCTCCCTTCGGG |
| | | AAGCGTGGCGCTTTCTCATAGCTCACGCTGTAGGTATCTCAGTTCGGTGTAGGTCGTTCGCTCCAAGCTGGGCTGTGTGCA |
| | | CGAACCCCCCGTTCAGCCCGACCGCTGCGCCTTATCCGGTAACTATCGTCTTGAGTCCAACCCGGTAAGACACGACTTATC |
| | | GCCACTGGCAGCAGCCACTGGTAACAGGATTAGCAGAGCGAGGTATGTAGGCGGTGCTACAGAGTTCTTGAAGTGGTGGC |
| | | CTAACTACGGCTACACTAGAAGAACAGTATTTGGTATCTGCGCTCTGCTGAAGCCAGTTACCTTCGGAAAAAGAGTTGGTAG |
| | | CTCTTGATCCGGCAAACAAACCACCGCTGGTAGCGGTGGTTTTTTTGTTTGCAAGCAGCAGATTACGCGCAGAAAAAAAGG |
| | | ATCTCAAGAAGATCCTTTGATCTTTTCTACGGGGTCTGACGCTCAGTGGAACGAAAACTCACGTTAAGGGATTTTGGTCATG |
| | | AGATTATCAAAAAGGATCTTCACCTAGATCCTTTTAAATTAAAAATGAAGTTTTAAATCAATCTAAAGTATATATGAGTAAACTT |
| | | GGTCTGACAGTTACCAATGCTTAATCAGTGAGGCACCTATCTCAGCGATCTGTCTATTTCGTTCATCCATAGTTGCCTGACTC |
| | | CCCGTCGTGTAGATAACTACGATACGGGAGGGCTTACCATCTGGCCCCAGTGCTGCAATGATACCGCGAGAACCACGCTC |
| | | ACCGGCTCCAGATTTATCAGCAATAAACCAGCCAGCCGGAAGGGCCGAGCGCAGAAGTGGTCCTGCAACTTTATCCGCCT |
| | | CCATCCAGTCTATTAATTGTTGCCGGGAAGCTAGAGTAAGTAGTTCGCCAGTTAATAGTTTGCGCAACGTTGTTGCCATTGC |
| | | TACAGGCATCGTGGTGTCACGCTCGTCGTTTGGTATGGCTTCATTCAGCTCCGGTTCCCAACGATCAAGGCGAGTTACATG |
| | | ATCCCCCATGTTGTGCAAAAAAGCGGTTAGCTCCTTCGGTCCTCCGATCGTTGTCAGAAGTAAGTTGGCCGCAGTGTTATCA |
| | | CTCATGGTTATGGCAGCACTGCATAATTCTCTTACTGTCATGCCATCCGTAAGATGCTTTTCTGTGACTGGTGAGTACTCAAC |
| | | CAAGTCATTCTGAGAATAGTGTATGCGGCGACCGAGTTGCTCTTGCCCGGCGTCAATACGGGATAATACCGCGCCACATAG |
| | | CAGAACTTTAAAAGTGCTCATCATTGGAAAACGTTCTTCGGGGCGAAAACTCTCAAGGATCTTACCGCTGTTGAGATCCAGT |
| | | TCGATGTAACCCACTCGTGCACCCAACTGATCTTCAGCATCTTTTACTTTCACCAGCGTTTCTGGGTGAGCAAAAACAGGAA |
| | | GGCAAAATGCCGCAAAAAAGGGAATAAGGGCGACACGGAAATGTTGAATACTCATACTCTTCCTTTTTCAATATTATTGAAG |
| | | CATTTATCAGGGTTATTGTCTCATGAGCGGATACATATTTGAATGTATTTAGAAAAATAAACAAATAGGGGTTCCGCGCACAT |
| | | TTCCCCGAAAAGTGCCAC |
| SEQ | 18 | AGCTTATTTTTTGTATACTGTTTTGTGATAGCACGAAGTTTTTCCACGGTATCTTGTTAAAAATATATATTTGTGGCGGGCTTA |
| ID | | CCTACATCAAATTAATAAGAGACTAATTATAAACTAAACACACAAGCAAGCTACTTTAGGGTAAAAGTTTATAAATGCTTTTGA |
| NO | | CGTATAAACGTTGCTTGTATTTATTATTACAATTAAAGGTGGATAGAAAACCTAGAGACTAGTTAGAAACTAATCTCAGGTTTG |
| | | CGTTAAACTAAATCAGAGCCCGAGAGGTTAACAGAACCTAGAAGGGGACTAGATATCCGGGTAGGGAAACAAAAAAAAAAA |
| | | ACAAGACAGCCACATATTAGGGAGACTAGTTAGAAGCTAGTTCCAGGACTAGGAAAATAAAAGACAATGATACCACAGTCTA |
| | | GTTGACAACTAGATAGATTCTAGATTGAGGCCAAAGTCTCTGAGATCCAGGTTAGTTGCAACTAATACTAGTTAGTATCTAGT |
| | | CTCCTATAACTCTGAAGCTAGAATAACTTACTACTATTATCCTCACCACTGTTCAGCTGCGCAAACGGAGTGATTGCAAGGT |
| | | GTTCAGAGACTAGTTATTGACTAGTCAGTGACTAGCAATAACTAACAAGGTATTAACCTACCATGTCTGCCATCACCCTGCAC |
| | | TTCCTCGGGCTCAGCAGCCTTTTCCTCCTCATTTTCATGCTCATTTTCCTTGTTTAAGACTGTGACTAGTCAAAGACTAGTCC |
| | | AGAACCACAAAGGAGAAATGTCTTACCACTTTCTTCATTGCTTGTCTCTTTTGCATTATCCATGTCTGCAACTAGTTAGAGTCT |
| | | AGTTAGTGACTAGTCCGACGAGGACTTGCTTGTCTCCGGATTGTTGGAGGAACTCTCCAGGGCCTCAAGATCCACAACAGA |
| | | GCCTTCTAGAAGACTGGTCAATAACTAGTTGGTCTTTGTCTGAGTCTGACTTACGAGGTTGCATACTCGCTCCCTTTGCCTC |
| | | GTCAATCGATGAGAAAAAGCGCCAAAACTCGCAATATGGCTTTGAACCACACGGTGCTGAGACTAGTTAGAATCTAGTCCCA |
| | | AACTAGCTTGGATAGCTTACCTTTGCCCTTTGCGTTGCGACAGGTCTTGCAGGGTATGGTTCCTTTCTCACCAGCTGATTTA |
| | | GCTGCCTTGCTACCCTCACGGCGGATCTGCATAAAGAGTGGCTAGAGGTTATAAATTAGCACTGATCCTAGGTACGGGGCT |
| | | GAATGTAACTTGCCTTTCCTTTCTCATCGCGCGGCAAGACAGGCTTGCTCAAATTCCTACCAGTCACAGGGGTATGCACGG |
| | | CGTACGGACCACTTGAACTAGTCACAGATTAGTTAGCAACTAGTCTGCATTGAATGGCTGTACTTACGGGCCCTCGCCATTG |
| | | TCCTGATCATTTCCAGCTTCACCCTCGTTGCTGCAAAGTAGTTAGTGACTAGTCAAGGACTAGTTGAAATGGGAGAAGAAAC |
| | | TCACGAATTCTCGACACCCTTAGTATTGTGGTCCTTGGACTTGGTGCTGCTATATATTAGCTAATACACTAGTTAGACTCACA |
| | | GAAACTTACGCAGCTCGCTTGCGCTTCTTGGTAGGAGTCGGGGTTGGGAGAACAGTGCCTTCAAACAAGCCTTCATACCAT |
| | | GCTACTTGACTAGTCAGGGACTAGTCACCAAGTAATCTAGATAGGACTTGCCTTTGGCCTCCATCAGTTCCTTCATAGTGGG |
| | | AGGTCCATTGTGCAATGTAAACTCCATGCCGTGGGAGTTCTTGTCCTTCAAGTGCTTGACCAATATGTTTCTGTTGGCAGAG |
| | | GGAACCTGTCAACTAGTTAATAACTAGTCAGAAACTAGTATAGCAGTAGACTCACTGTACGCTTGAGGCATCCCTTCACTCG |
| | | GCAGTAGACTTCATATGGATGGATATCAGGCACGCCATTGTCGTCCTGTGGACTAGTCAGTAACTAGGCTTAAAGCTAGTCG |
| | | GGTCGGCTTACTATCTTGAAATCCGGCAGCGTAAGCTCCCCGTCCTTAACTGCCTCGAGATAGTGACAGTACTCTGGGGAC |
| | | TTTCGGAGATCGTTATCGCGAATGCTCGGCATACTAATCGTTGACTAGTCTTGGACTAGTCCCGAGCAAAAAGGATTGGAG |
| | | GAGGAGGAGGAAGGTGAGAGTGAGACAAAGAGCGAAATAAGAGCTTCAAAGGCTATCTCTAAGCAGTATGAAGGTTAAGTA |
| | | TCTAGTTCTTGACTAGATTTAAAAGAGATTTCGACTAGTTATGTACCTGGAGTTTGGATATAGGAATGTGTTGTGGTAACGAA |
| | | ATGTAAGGGGGAGGAAAGAAAAAGTCGGTCAAGAGGTAACTCTAAGTCGGCCATTCCTTTTTGGGAGGCGCTAACCATAAA |
| | | CGGCATGGTCGACTTAGAGTTAGCTCAGGGAATTTAGGGAGTTATCTGCGACCACCGAGGAACGGCGGAATGCCAAAGAA |
| | | TCCCGATGGAGCTCTAGCTGGCGGTTGACAACCCCACCTTTTGGCGTTTCTGCGGCGTTGCAGGCGGGACTGGATACTTC |
| | | GTAGAACCAGAAAGGCAAGGCAGAACGCGCTCAGCAAGAGTGTTGGAAGTGATAGCATGATGTGCCTTGTTAACTAGGTCA |
| | | AAATCTGCAGTATGCTTGATGTTATCCAAAGTGTGAGAGAGGAAGGTCCAAACATACACGATTGGGAGAGGGCCTAGGTAT |
| | | AAGAGTTTTTGAGTAGAACGCATGTGAGCCCAGCCATCTCGAGGAGATTAAACACGGGCCGGCATTTGATGGCTATGTTAG |
| | | TACCCCAATGGAAAGCCTGAGAGTCCAGTGGTCGCAGATAACTCCCTAAATTCCCTGAGCTAACTCTAAGTCGACCATGCC |
| | | GTTTATGGTTAGCGCCTCCCAAAAAGGAATGGCCGACTTAGAGTTACCTCTTGACCGACTTTTTCTTTCCTCCCCCTTACATT |
| | | TCGTTACCACAACACATTCCTATATCCAAACTCCAGGTACATAACTAGTCGAAATCTCTTTTAAATCTAGTCAAGAACTAGATA |
| | | CTTAACCTTCATACTGCTTAGAGATAGCCTTTGAAGCTCTTATTTCGCTCTTTGTCTCACTCTCACCTTCCTCCTCCTCCTCCA |
| | | ATCCTTTTTGCTCGGGACTAGTCCAAGACTAGTCAACGATTAGTATGCCGAGCATTCGCGATAACGATCTCCGAAAGTCCCC |
| | | AGAGTACTGTCACTATCTCGAGGCAGTTAAGGACGGGGAGCTTACGCTGCCGGATTTCAAGATAGTAAGCCGACCCGACTA |
| | | GCTTTAAGCCTAGTTACTGACTAGTCCACAGGACGACAATGGCGTGCCTGATATCCATCCATATGAAGTCTACTGCCGAGTG |
| | | AAGGGATGCCTCAAGCGTACAGTGAGTCTACTGCTATACTAGTTTCTGACTAGTTATTAACTAGTTGACAGGTTCCCTCTGC |
| | | CAACAGAAACATATTGGTCAAGCACTTGAAGGACAAGAACTCCCACGGCATGGAGTTTACATTGCACAATGGACCTCCCACT |
| | | ATGAAGGAACTGATGGAGGCCAAAGGCAAGTCCTATCTAGATTACTTGGTGACTAGTCCCTGACTAGTCAAGTAGCATGGTA |
| | | TGAAGGCTTGTTTGAAGGCACTGTTCTCCCAACCCCGACTCCTACCAAGAAGCGCAAGCGAGCTGCGTAAGTTTCTGTGAG |
| | | TCTAACTAGTGTATTAGCTAATATATAGCAGCACCAAGTCCAAGGACCACAATACTAAGGGTGTCGAGAATTCGTGAGTTTCT |
| | | TCTCCCATTTCAACTAGTCCTTGACTAGTCACTAACTACTTTGCAGCAACGAGGGTGAAGCTGGAAATGATCAGGACAATGG |
| | | CGAGGGCCCGTAAGTACAGCCATTCAATGCAGACTAGTTGCTAACTAATCTGTGACTAGTTCAAGTGGTCCGTACGCCGTG |
| | | CATACCCCTGTGACTGGTAGGAATTTGAGCAAGCCTGTCTTGCCGCGCGATGAGAAAGGAAAGGCAAGTTACATTCAGCCC |
| | | CGTACCTAGGATCAGTGCTAATTTATAACCTCTAGCCACTCTTTATGCAGATCCGCCGTGAGGGTAGCAAGGCAGCTAAATC |
| | | AGCTGGTGAGAAAGGAACCATACCCTGCAAGACCTGTCGCAACGCAAAGGGCAAAGGTAAGCTATCCAAGCTAGTTTGGGA |
| | | CTAGATTCTAACTAGTCTCAGCACCGTGTGGTTCAAAGCCATATTGCGAGTTTTGGCGCTTTTTCTCATCGATTGACGAGGC |
| | | AAAGGGAGCGAGTATGCAACCTCGTAAGTCAGACTCAGACAAAGACCAACTAGTTATTGACCAGTCTTCTAGAAGGCTCTGT |
| | | TGTGGATCTTGAGGCCCTGGAGAGTTCCTCCAACAATCCGGAGACAAGCAAGTCCTCGTCGGACTAGTCACTAACTAGACT |
| | | CTAACTAGTTGCAGACATGGATAATGCAAAAGAGACAAGCAATGAAGAAAGTGGTAAGACATTTCTCCTTTGTGGTTCTGGA |
| | | CTAGTCTTTGACTAGTCACAGTCTTAAACAAGGAAAATGAGCATGAAAATGAGGAGGAAAAGGCTGCTGAGCCCGAGGAAG |
| | | TGCAGGGTGATGGCAGACATGGTAGGTTAATACCTTGTTAGTTATTGCTAGTCACTGACTAGTCAATAACTAGTCTCTGAAC |
| | | ACCTTGCAATCACTCCGTTTGCGCAGCTGAACAGTGGTGAGGATAATAGTAGTAAGTTATTCTAGCTTCAGAGTTATAGGAG |
| | | ACTAGATACTAACTAGTATTAGTTGCAACTAACCTGGATCTCAGAGACTTTGGCCTCAATCTAGAATCTATCTAGTTGTCAAC |
| | | TAGACTGTGGTATCATTGTCTTTTATTTTCCTAGTCCTGGAACTAGCTTCTAACTAGTCTCCCTAATATGTGGCTGTCTTGTTT |
| | | TTTTTTTTTGTTTCCCTACCCGGATATCTAGTCCCCTTCTAGGTTCTGTTAACCTCTCGGGCTCTGATTTAGTTTAACGCAAAC |
| | | CTGAGATTAGTTTCTAACTAGTCTCTAGGTTTTCTATCCACCTTTAATTGTAATAATAAATACAAGCAACGTTTATACGTCAAA |
| | | AGCATTTATAAACTTTTACCCTAAAGTAGCTTGCTTGTGTGTTTAGTTTATAATTAGTCTCTTATTAATTTGATGTAGGTAAGCC |
| | | CGCCACAAATATATATTTTTAACAAGATACCGTGGAAAAACTTCGTGCTATCACAAAACAGTATACAAAAAATAAGCT |
| SEQ | 19 | UUUCGCCCGCAUGGAUUCGCGAGACGA |
| ID | | |
| NO | | |
| SEQ | 20 | UUUGGAGGACGCCGUGGUCGGUCGGGA |
| ID | | |
| NO | | |
| SEQ | 21 | UUUGAGGCACAGAAACCCAAUAGUCAA |
| ID | | |
| NO | | |
| SEQ | 22 | UUUCGCCACGGAGCUUUACAGGCACUG |
| ID | | |
| NO | | |
| SEQ | 23 | UUUCCAUAAUCCGCCAUCAUGAAGUUU |
| ID | | |
| NO | | |
| SEQ | 24 | UUUGCCAACAAGAACACCAACGUCUAA |
| ID | | |
| NO | | |
| SEQ | 25 | UUUCCCCCCAUGAAGAACUAUUGCAUC |
| ID | | |
| NO | | |
| SEQ | 26 | UUUAUGAGGAAAAGGGAAAAAAGGGAA |
| ID | | |
| NO | | |
| SEQ | 27 | UUUCGCCACAAACGUCCUGCUGCUGGC |
| ID | | |
| NO | | |
| SEQ | 28 | UUUGGGUCUUGGUGGAGAUCGUGCCAC |
| ID | | |
| NO | | |
| SEQ | 29 | UUUCUUCUCAUCAGCCCCAUCAGCCUU |
| ID | | |
| NO | | |
| SEQ | 30 | UUUCACGUUCCAUCAUUGAUCCCAUAG |
| ID | | |
| NO | | |
| SEQ | 31 | UUUCCUAAGCUGCUUGAUUGAAGGAUG |
| ID | | |
| NO | | |
| SEQ | 32 | UUUAGAGUAUACAGCUUCGGACUCAAC |
| ID | | |
| NO | | |
| SEQ | 33 | GGCATAGCAACGCAAAGCCTCAGATCAAAACGGCATCGCTCCCACCCGGACGCTCAGCCTCTGAGACTCTTGACAAGGGT |
| ID | | CCGCAAGACAGCCCGAAGAACAGACGCACGCGCTCGATCTGAGCACACACCGAGGCCCTCTTTTCGGCGCCCAGTCCTGG |
| NO | | GTAATACAATTGTATCTCGCTGCCTCCAACTCGACGCATACATACCTACATACCACCTCACTCTACCTACCTCCTCCGGATAC |
| | | TACTTTGCAATACCTCCTCGTCTGCGCTGGTCGGCTCTTTGACAAACCAAGCCTGCCGATTGCTAACCATACCACCCTTTAC |
| | | CTACTGGAGCCCTCTGCTACGAGCACACAGTTTAGAGTAACCGTACGAGTCCGTACCTCCAACGCTCAAGCTCGAGAGTTG |
| | | TGCCTTGCCTGCTCTCTTGACTGAAGCCAGAGGACCCGCCCGGCCAGAGTGCGGCGGTTCCCCCAATTTCACTCATCCAA |
| | | GCACCGGTCGGCGCCAGctttccctctcttcctctctctctctctctGTTCTTTTTTTTTTCTCCCCCATTAGCCTTCTTACTTTCGCCTGTCACA |
| | | GGCCTACGACGTCAAGCAGAGAGAATTGCAGGGGTACTCCGTGCTGCTCTTACTCTTTTTCTCTCCCTTGATCCCCTCTCGT |
| | | CACCCCTCCACTCGGACTCGTATCATCCTACGTACTTACCAGACAGCATTCGGCGTGCACATCAAAGGCGTAGCTTTCCCT |
| | | GTCATTCATCCCACTTTCATCCAGCAAGCGTCGTCCGAAGGGCTTCCCGCAATCCATGGCAGCTGTCGGACCGCTTGCGAT |
| | | GCACCCGACCGTCACAGAACACGATTACCGCTTCCCGCGAAGACCCGATCCTCATCGAAGTACCGACCGCAGCTGCAGCT |
| | | GTGGAGCCGCATTCCCGATTCGGGCCGGATTGGTCAAGATTTGCGTCCGAGGTGCCGTCTATCATTCTAGCTTGCGGTCCT |
| | | GGGCTTGTGACTGGTCGCGAGCTGCCACTAAGTGGGGCAGTACCATTTTATCGGACCCATCCAGCTATGGGACCCACTCG |
| | | CAAATTTTTACATCATTTTTTTTTGCTCAGTAACGGCCACCTTTTGTAAAGCGTAACCAGCAAACAAATTGCAATTGGCCCG |
| | | TAGCAAGGTAGTCAGGGCTTATCGTGATGGAGGAGAAGGCTATATCAGCCTCAAAAATATGTTGCCAGCTGGCGGAAGCCC |
| | | GGAAGGTAAGTGGATTCTTCGCCGTGGCTGGAGCAACCGGTGGATTCCAGCGTCTCCGACTTGGACTGAGCAATTCAGCG |
| | | TCACGGATTCACGATAGACAGCTCAGACCGCTCCACGGCTGGCGGCATTATTGGTTAACCCGGAAACTCAGTCTCCTTGGC |
| | | CCCGTCCCGAAGGGACCCGACTTACCAGGCTGGGAAAGCCAGGGATAGAATACACTGTACGGGCTTCGTACGGGAGGTTC |
| | | GGCGTAGGGTTGTTCCCAAGTTTTACACACCCCCCAAGACAGCTAGCGCACGAAAGACGCGGAGGGTTTGGTGAAAAAAG |
| | | GGCGAAAATTAAGCGGGAGACGTATTTAGGTGCTAGGGCCGGTTTCCTCCCCATTTTTCTTCGGTTCCCTTTCTCTCCTGGA |
| | | AGACTTTCTCTCTCTCTCTTCTTCTCTTCTTCCATCCTCAGTCCATCTTCCTTTCCCATCATCCATCTCCTCACCCCCATCTCA |
| | | ACTCCATCACATCACAATCGATATGAAAAAGCCTGAACTCACCGCGACGTCTGTCGAGAAGTTTCTGATCGAAAAGTTCGAC |
| | | AGCGTCTCCGACCTGATGCAGCTCTCGGAGGGCGAAGAATCTCGTGCTTTCAGCTTCGATGTAGGAGGGCGTGGATATGT |
| | | CCTGCGGGTAAATAGCTGCGCCGATGGTTTCTACAAAGATCGTTATGTTTATCGGCACTTTGCATCGGCCGCGCTCCCGAT |
| | | TCCGGAAGTGCTTGACATTGGGGAGTTCAGCGAGAGCCTGACCTATTGCATCTCCCGCCGTGCACAGGGTGTCACGTTGC |
| | | AAGACCTGCCTGAAACCGAACTGCCCGCTGTTCTGCAGCCGGTCGCGGAGGCCATGGATGCGATCGCTGCGGCCGATCTT |
| | | AGCCAGACGAGCGGGTTCGGCCCATTCGGACCGCAAGGAATCGGTCAATACACTACATGGCGTGATTTCATATGCGCGATT |
| | | GCTGATCCCCATGTGTATCACTGGCAAACTGTGATGGACGACACCGTCAGTGCGTCCGTCGCGCAGGCTCTCGATGAGCT |
| | | GATGCTTTGGGCCGAGGACTGCCCCGAAGTCCGGCACCTCGTGCACGCGGATTTCGGCTCCAACAATGTCCTGACGGACA |
| | | ATGGCCGCATAACAGCGGTCATTGACTGGAGCGAGGCGATGTTCGGGGATTCCCAATACGAGGTCGCCAACATCTTCTTCT |
| | | GGAGGCCGTGGTTGGCTTGTATGGAGCAGCAGACGCGCTACTTCGAGCGGAGGCATCCGGAGCTTGCAGGATCGCCGCG |
| | | GCTCCGGGCGTATATGCTCCGCATTGGTCTTGACCAACTCTATCAGAGCTTGGTTGACGGCAATTTCGATGATGCAGCTTG |
| | | GGCGCAGGGTCGATGCGACGCAATCGTCCGATCCGGAGCCGGGACTGTCGGGCGTACACAAATCGCCCGCAGAAGCGCG |
| | | GCCGTCTGGACCGATGGCTGTGTAGAAGTACTCGCCGATAGTGGAAACCGACGCCCCAGCACTCGTCCGAGGGCAAAGGA |
| | | ATAGGGATCCACTTAACGTTACTGAAATCATCAAACAGCTTGACGAATCTGGATATAAGATCGTTGGTGTCGATGTCAGCTC |
| | | CGGAGTTGAGACAAATGGTGTTCAGGATCTCGATAAGATACGTTCATTTGTCCAAGCAGCAAAGAGTGCCTTCTAGTGATTT |
| | | AATAGCTCCATGTCAACAAGAATAAAACGCGTTTTCGGGTTTACCTCTTCCAGATACAGCTCATCTGCAATGCATTAATGCAT |
| | | TGACTGCAACCTAGTAACGCCTTCAGGCTCCGGCGAAGAGAAGAATAGCTTAGCAGAGCTATTTTCATTTTCGGGAGACGA |
| | | GATCAAGCAGATCAACGGTCGTCAAGGCGGCCGCAGGCTAGGTGGAGGCTCAGTGATGATAAGTCTGCGATGGTGGATGC |
| | | ATGTGTCATGAGACAGCGTAGGCATAGCAAGAGCGAGGGCACTTCATCGTCGGCTATGGATCTCGACGGCCCTGGAGACT |
| | | CTATTGGGCGACAGCTTGGCGCAACTCCCAGCATGCCTTCCATCTCTAGCACTATGCTTTCGAATGGAAGCTTCACCATGG |
| | | GTCAGCGCTCCATGATGGGATCAAGCATGGTGTCCATGGGTGGTTCCCAGGGCAACCTTGTGAACAGCGCCGGTGGCTCG |
| | | TCAGGCCCTCAGGAATGGGAGTGGTTGACTATGAGTCTTTGAAGCGGCCGGCGAGATGGCGAATTTGACACGGGATCGTT |
| | | GATGCTAGACGAAAAGATACGGCGTGCGATCAGTGTTTGGGTCAACCTTGAGGAATGGTGGCAAGGCTTGAGAAGGGGCC |
| | | GACCATGGCCTCACAATATGGGCTGGCTGGCATTGCTGAGATGAGCTGATGGCGGCGCGAAGGATATGGGCGCTTTAAGT |
| | | AATGGGCATCTTCATGGCGTGTTGGTTCAAGAAATGATACCTGGGTCTTTTGTGTGTTTTTTTGGCTTGCACGTACGGACTA |
| | | CCTTTCGGAGGTTGACAATGTGTTCTATCTTTACTCTTTTATTTCTTTCTGCTCTGCCTGCGCTTGGATATGGGCGGCAGTCT |
| | | TCGAAGAACAGCCTCGGTGCGATAACCAGCAACTATTTTATACAGACATGGAGAGCGCCCGTCAAGGAACTACCTATTTATG |
| | | AATGCCAGGGGCGGTGTCCAAGGGAGCCACGACGACCGGTCCCGCGCTTACGACCTGATGTCGACATAATCTGGAGTATG |
| | | TT |
| SEQ | 34 | ACGGTTGCAATGCAGGGGTACTGAGCTTGGACATAACTGTTCCGTACCCCACCTCTTCTCAACCTTTGGCGTTTCCCTGATT |
| ID | | CAGCGTACCCGTACAAGTCGTAATCACTATTAACCCAGACTGACCGGACGTGTTTTGCCCTTCATTTGGAGAAATAATGTCA |
| NO | | TTGCGATGTGTAATTTGCCTGCTTGACCGACTGGGGCTGTTCGAAGCCCGAATGTAGGATTGTTATCCGAACTCTGCTCGTA |
| | | GAGGCATGTTGTGAATCTGTGTCGGGCAGGACACGCCTCGAAGGTTCACGGCAAGGGAAACCACCGATAGCAGTGTCTAG |
| | | TAGCAACCTGTAAAGCCGCAATGCAGCATCACTGGAAAATACAAACCAATGGCTAAAAGTACATAAGTTAATGCCTAAAGAA |
| | | GTCATATACCAGCGGCTAATAATTGTACAATCAAGTGGCTAAACGTACCGTAATTTGCCAACGGCTTGTGGGGTTGCAGAAG |
| | | CAACGGCAAAGCCCCACTTCCCCACGTTTGTTTCTTCACTCAGTCCAATCTCAGCTGGTGATCCCCCAATTGGGTCGCTTGT |
| | | TTGTTCCGGTGAAGTGAAAGAAGACAGAGGTAAGAATGTCTGACTCGGAGCGTTTTGCATACAACCAAGGGCAGTGATGGA |
| | | AGACAGTGAAATGTTGACATTCAAGGAGTATTTAGCCAGGGATGCTTGAGTGTATCGTGTAAGGAGGTTTGTCTGCCGATAC |
| | | GACGAATACTGTATAGTCACTTCTGATGAAGTGGTCCATATTGAAATGTAAGTCGGCACTGAACAGGCAAAAGATTGAGTTG |
| | | AAACTGCCTAAGATCTCGGGCCCTCGGGCCTTCGGCCTTTGGGTGTACATGTTTGTGCTCCGGGCAAATGCAAAGTGTGGT |
| | | AGGATCGAACACACTGCTGCCTTTACCAAGCAGCTGAGGGTATGTGATAGGCAAATGTTCAGGGGCCACTGCATGGTTTCG |
| | | AATAGAAAGAGAAGCTTAGCCAAGAACAATAGCCGATAAAGATAGCCTCATTAAACGGAATGAGCTAGTAGGCAAAGTCAGC |
| | | GAATGTGTATATATAAAGGTTCGAGGTCCGTGCCTCCCTCATGCTCTCCCCATCTACTCATCAACTCAGATCCTCCAGGAGA |
| | | CTTGTACACCATCTTTTGAGGCACAGAAACCCAATAGTCAACCGCGGACTGCGCATCATGTATCGGAAGTTGGCCGTCATCT |
| | | CGGCCTTCTTGGCCACAGCTCGTGCTCAGTCGGCCTGCACTCTCCAATCGGAGACTCACCCGCCTCTGACATGGCAGAAAT |
| | | GCTCGTCTGGTGGCACGTGCACTCAACAGACAGGCTCCGTGGTCATCGACGCCAACTGGCGCTGGACTCACGCTACGAAC |
| | | AGCAGCACGAACTGCTACGATGGCAACACTTGGAGCTCGACCCTATGTCCTGACAACGAGACCTGCGCGAAGAACTGCTG |
| | | TCTGGACGGTGCCGCCTACGCGTCCACGTACGGAGTTACCACGAGCGGTAACAGCCTCTCCATTGGCTTTGTCACCCAGT |
| | | CTGCGCAGAAGAACGTTGGCGCTCGCCTTTACCTTATGGCGAGCGACACGACCTACCAGGAATTCACCCTGCTTGGCAAC |
| | | GAGTTCTCTTTCGATGTTGATGTTTCGCAGCTGCCGTAAGTGACTTACCATGAACCCCTGACGCTATCTTCTTGTTGGCTCC |
| | | CAGCTGACTGGCCAATTCAAGGTGCGGCTTGAACGGAGCTCTCTACTTCGTGTCCATGGACGCGGATGGTGGCGTGAGCA |
| | | AGTATCCCACCAACACCGCTGGCGCCAAGTACGGCACGGGGTACTGTGACAGCCAGTGTCCCCGCGATCTGAAGTTCATC |
| | | AATGGCCAGGCCAACGTTGAGGGCTGGGAGCCGTCATCCAACAACGCGAACACGGGCATTGGAGGACACGGAAGCTGCT |
| | | GCTCTGAGATGGATATCTGGGAGGCCAACTCCATCTCCGAGGCTCTTACCCCCCACCCTTGCACGACTGTCGGCCAGGAG |
| | | ATCTGCGAGGGTGATGGGTGCGGCGGAACTTACTCCGATAACAGATATGGCGGCACTTGCGATCCCGATGGCTGCGACTG |
| | | GAACCCATACCGCCTGGGCAACACCAGCTTCTACGGCCCTGGCTCAAGCTTTACCCTCGATACCACCAAGAAATTGACCGT |
| | | TGTCACCCAGTTCGAGACGTCGGGTGCCATCAACCGATACTATGTCCAGAATGGCGTCACTTTCCAGCAGCCCAACGCCGA |
| | | GCTTGGTAGTTACTCTGGCAACGAGCTCAACGATGATTACTGCACAGCTGAGGAGGCAGAATTCGGCGGATCCTCTTTCTC |
| | | AGACAAGGGCGGCCTGACTCAGTTCAAGAAGGCTACCTCTGGCGGCATGGTTCTGGTCATGAGTCTGTGGGATGATGTGA |
| | | GTTTGATGGACAAACATGCGCGTTGACAAAGAGTCAAGCAGCTGACTGAGATGTTACAGTACTACGCCAACATGCTGTGGC |
| | | TGGACTCCACCTACCCGACAAACGAGACCTCCTCCACACCCGGTGCCGTGCGCGGAAGCTGCTCCACCAGCTCCGGTGTC |
| | | CCTGCTCAGGTCGAATCTCAGTCTCCCAACGCCAAGGTCACCTTCTCCAACATCAAGTTCGGACCCATTGGCAGCACCGGC |
| | | AACCCTAGCGGCGGCAACCCTCCCGGCGGAAACCCGCCTGGCACCACCACCACCCGCCGCCCAGCCACTACCACTGGAT |
| | | CCGTGGCTGTGGAGAAGCGCGACGTCCAGCTCGTCGAGAGCGGCGGCGGCCTCGTCCAGGCTGGCGGCAGCCTCCGCC |
| | | TCTCCTGCGCCGCCTCGCGCAGCATCTTCAGCATCAACGCCATGGACTGGTATCGACAGGCCCCTGGCAAGCAGCGCGAG |
| | | TGGGTCGCCGGCATCACCCGTGGCGGCACCACGAAGTACGCCGACTCCGTCAAGGGCCGCTTCACCATCAGCCGCGATA |
| | | ACGCCAAGAAGAAGGTCTATCTCCAGATGAACTCGCTCAAGCCCGAGGACACCGCCGTCTACTACTGCAACGTCCTCCGC |
| | | GGCGAGCAGCCCTGGACGCGCGACTACTGGGGCCAGGGCACCCAGGTCACCGTCAGCAGCTAAAGCTCCGTGGCGAAAG |
| | | CCTGACGCACCGGTAGATTCTTGGTGAGCCCGTATCATGACGGCGGCGGGAGCTACATGGCCCCGGGTGATTTATTTTTTT |
| | | TGTATCTACTTCTGACCCTTTTCAAATATACGGTCAACTCATCTTTCACTGGAGATGCGGCCTGCTTGGTATTGCGATGTTGT |
| | | CAGCTTGGCAAATTGTGGCTTTCGAAAACACAAAACGATTCCTTAGTAGCCATGCATTTTAAGATAACGGAATAGAAGAAAG |
| | | AGGAAATTAAAAAAAAAAAAAAAACAAACATCCCGTTCATAACCCGTAGAATCGCCGCTCTTCGTGTATCCCAGTACCACGG |
| | | CAAAGGTATTTCATGATCGTTCAATGTTGATATTGTTCCCGCCAGTATGGCTCCACCCCCATCTCCGCGAATCTCCTCTTCTC |
| | | GAACGCGGTAGTGGCGCGCCAATTGGTAATGACCCATAGGGAGACAAACAGCATAATAGCAACAGTGGAAATTAGTGGCG |
| | | CAATAATTGAGAACACAGTGAGACCATAGCTGGCGGCCTGGAAAGCACTGTTGGAGACCAACTTGTCCGTTGCGAGGCCAA |
| | | CTTGCATTGCTGTCAAGACGATGACAACGTAGCCGAGGACCGTCACAAGGGACGCAAAGTTGTCGCGGATGAGGTCTCCG |
| | | TAGATGGCATAGCCGGCAATCCGAGAGTAGCCTCTCAACAGGTGGCCTTTTCGAAACCGGTAAACCTTGTTCAGACGTCCT |
| | | AGCCGCAGCTCACCGTACCAGTATCGAGGATTGACGGCAGAATAGCAGTGGCTCTCCAGGATTTGACTGGACAAAATCTTC |
| | | CAGTATTCCCAGGTCACAGTGTCTGGCAGAAGTCCCTTCTCGCGTGCGAGTCGAAAGTCGCTATAGTGCGCAATGAGAGCA |
| | | CAGTAGGAGAATAGGAACCCGCGAGCACATTGTTCAATCTCCACATGAATTGGATGACTGCTGGGCAGAATGTGCTGCCTC |
| | | CAAAATCCTGCGTCCAACAGATACTCTGGCAGGGGCTTCAGATGAATGCCTCTGGGCCCCCAGATAAGATGCAGCTCTGGA |
| | | TTCTCGGTTACGATGATATCGCGAGAGAGCACGAGTTGGTGATGGAGGGGACGAGGAGGCATAGGTCGGCCGCAGGCCC |
| | | ATAACCAGTCTTGCACAGCATTGATCTTCCTCACGAGGAGCTCCTGAT |
| SEQ | 35 | CTCAAGGACTACTACTGCCAATCTTACCTCTATCTCTAGTCATCTTGTTCCAACGCTTTGATAGAGTAAACGGTCAGTAGTAG |
| ID | | TTGACATCTCTCGCCTTGACTGAATCTGCCGAGTCAGACGCACTGCAATCTTACAACACCTGAGTCACCTTATTCTTATTTTG |
| NO | | ACTCAGAGCTTAAAGCCGCCAGATGCCGGTCCACAGAGCTCTCTCTTCCGTACAAATGATCCCAAGCAAAGCAAGGAGAGC |
| | | ATCATCATATTATCCATGTGATCCAACAGAGAGAGACAACTTTCGTGTACACTCACCTTTAAAAAAAAACGCCATGCATGAGA |
| | | AACGAGAAATCAATGCCGTCATCCAAAGTCAACCTCATCGTCAGGTGTGCCAATGGACGAGCAGAGCTGAAATGCAGCTGT |
| | | GACCAAGAAGCAGCGAGCAAGGGGGCAGAAAGGCACGGCCAACGCATATCTGCTGATGGCCTAATGTCTCGGTCCATCCC |
| | | TGGGGGGAATCGTCACCAAGGAGAATAAGAGTTGTCTCAGAAGAAAGAGAGAGAGAAACAAAACAGTGTCTCAGAGAAATG |
| | | CCCTCGTCTTCCCGCCCCCAGGTGCGGACTCCGCGCTTTTTTGCTTGTCCTCAATTGCTGCAAAAGACACGTCCTGATGCT |
| | | CTTGCTTGCCTCGAATCACATTCCTACACATTCCTGGCCAAGGCTTGGGCAGCCATCACGCGGATATTCGACGGCTCTATAT |
| | | GGCAATGATCCGTCAGTAAACGGAAGATGGGCCTTCCGCCATCGTGTAGAATCGCCATGATGCGGGTTGGAATCCAAAAGT |
| | | GCAGGGAGGTCGTTTCAGGCCGGAATGCCATGAGTATATAATACGCGAAAGGGTCGTGAATAATCCATCTTTCTCCCTTCC |
| | | AGTATCAAAGGGTTTCTTCTCTCCCGTCTTCTTTTGATATAAAAGGAGCAGAGAGCCACCGGAAGCTGATCTCGATTCGACA |
| | | TTTTCTCTTTTTTTTCTGCCGTAATAAGATTGAGCATCTAGTTAGTAGTTGGACATTGTCTTGGGATGCTGCTTGCGACTACCT |
| | | ACCTATGATACATGTATGGAAGGAAGCAGTCGGTTGCACGGCTTGGATCAGTAAACGGTCGGAATGTCACCAGAAGGGGG |
| | | GGAGGAAGGGGGTTTCAAACTGGATGACAGGGTAACGTTTCAATGTTCTCTCATGGGGCTAATGATGGTTGATGATGAAAG |
| | | AATCTCTTGTTTCGGCTTTTGGAAAGAATACGATAGTATTACCTACTAGTACTAGGCAGTCATCAATTAAGGGGGAGATATCA |
| | | ATAGGGATGTCGATCCGGGTTCTGGAATATTCAGTTTCTTCAGATCCATGGCTGCAAAGAGTTTGGCGACTGTCAACATTTA |
| | | TTCAACCTTTTTGGTGCCTTGTTGATTAATCTCCGTGAGATGCGGTAATCGTCAGCTAAAAACTATCATTGCTTGCATCTCAC |
| | | TAACACGACTGGGTCCTCCTCACCAAACGGCACATGTAGCTTAAATCCGTTGCGACTGCTGCAGCATCAAGAGTGCCAAAA |
| | | AAAGCCATTGAATCGCTTCGATTGAAATTCTGTTGACCAGATGGTACTACAGTTACGACGAATTCAAAAGTCTATGATTCGTA |
| | | TAAGCGTATCGAATTCCGGGTAAGTTGGACTGGCTTATTCGTCAGGCACCGGCTGCCAAGAAACGGCAACCTGCACGAAGC |
| | | AGCAAGAACTGCCCATGGCCTCGGACTTTGCTCATCAGGCCAGTCATGGACCGAGGCAGCCTCGTCAGATCATGCCATCTA |
| | | TTGGCCTGGACGAGAAACCTCTGCCACGTGGAGACCGGCTTTTGAGTCGGCTTGGAGGCTGTCAAGGACGCCTCGAGGGC |
| | | TGCCCAAGGATAAGATATACAAGAGGGACATCCATATACATATATAGGGCCAAATGGACGGCGAACCGGGCGCCGGGCCT |
| | | CACCAGCTCACCGTTGCCAACCTGCAAACCCTCAACCAGCAAGTCAAGCAGTTCCACCTCCTCCGGCACCGCAAGGCAATG |
| | | GCCTCCAACATCCTCGTCCTGGGCGCCGGCGAACT |
| SEQ | 36 | GCATAATATATCGAAGCCGCCACCGAAACATCATAGACAAACATAAAAGATGAGCTTCCTACATCAAACCGACAAACATCAT |
| ID | | CTTATAATAGATCATGATCGCAGATGCAGCATTAATAGCCTCTCGCACCAGAGCACACCAATCATTCAGCCATCGTGAACAA |
| NO | | CAACCCCTGCCTCCTTACAAAGAATCCTCAGCTCTCAACGCCAAAAGTCCTCCTCCAACCGGCAACTCCGCCGCCAGCCAA |
| | | CTGCTTTTCTCTCCTCCCATAGACTCTCTTTTAGATCCATTTCTCCAGCAAAAGAACAAAAAGCCTCCGGCCGGCAGGGGCC |
| | | GACATGCCCCTGGCTCTGCAAATCATGCCCCATCAGGAAGGTCGCAAATAGACGTGCAAAGATGAGGGAGCTAATATGCAT |
| | | GGTTTTGGGCATGAGCTGCGGGTCGCGGGATCTTTTGAATTGACTTGACGGACACGGGCAGCGATTAACAATGGAAAACTC |
| | | GAGGGAAGGCAAGAATCTTCTCCTCTTGTGGACAACAAACGGGGTATGAACAGAGCCCCCCCCTCCCCTTTGTTTGGGGCT |
| | | GTCGTATTATAGGTCTCGGCTGACAAGGCTTCGCCCTTTGATGCAAGCAGCCTGCCCAGAGAATCCTCTGCAATTTCCAAG |
| | | GCTGCAACAAACAATTCTCTCCTCGCCAATCCCGGGCCACACCGCACGCTCAACTCACCGACAAGGCTATCGCTCCGCAGC |
| | | CACGCCAAGTCCATAACAACAAACAGCGGCCGCTCCCCTCCAGAAAAAGAGGCAGGGATTAAGGCCGGCGAGATGCCATC |
| | | ACGGCATTGTGCTCCTCTCGGTAGGCTGAACCCTCCCTCCCCAGCCCCGGACACACTATCCGCTTATGCACAATGGAGGA |
| | | GGCACACTTGGCGAGGCGCTGTCCAGGGATTCGCGCATGCCGTGGTACTTTTTGTGTTGGCGACGAGAGTAGCTAGTAGA |
| | | TGGGCGCGCTGTGCGATGTTACCTTGAATAATAAACTAAAAAGGGGGGTGCTAGATCTCCTTGTACAAGAACATGATGACTT |
| | | GATCGATTGATGAAGGAAGTGAATTCGACGCGATCTCCATCGAAGCTTGAGGCATCAGGGACGATGACATCTAGGGAGCCA |
| | | CCGCCGAGGAAGAACCACCGAGAGAGTTTCAAGCACCCAGTCTACTTGGGGCACGATATGTACACATTAGAAATCCCCCCA |
| | | GCCTCTCGCTTCTAATTCCTTGTGCTGCCTCCTCTCCTGTCTTCTCTTCTTCCTCTTTAGTTTTCCTTGTCACGATCAGAAGC |
| | | CTCTCTCTCTCTCTCATTCTCTTTCTTGGAGTGAAGCAGATCTGAAGTATTCTCTCATGTTCGTCAGACATGAGCACCAGAGC |
| | | TTTCTACAGTATCATCTTGTGAGAGTACCTCGGCCAGAGAGTTTTCATATTGAAGCTTGGCCCACCATTTTCGAGCAGTCATT |
| | | GGCGCTTCCGTTCCCTTCCAATTGAGGCCTCTGATTGTCTTGATATAAAAGCACATTCGCAACTTTTGTTTTTGATAGGTGAG |
| | | CATTCAGTGGAAGAAGCGGCTCCAGGAGCAAATGAAGAACGAGGCGGCGGAGGAGGCGGCCTCTCCTCGCATGGCGGTT |
| | | AGTTGAAGGCGATAATAGCGTGAGGGAAGAGACCAACTTGGCTTGTTCTTTTCAAAGGACGTTGACTGTAACCTCCTAGATG |
| | | TCTTTCATGAAGTGACCAGAATGCTGTCGCCACATGGGGTCGAAGGCATCGGGAGGAGCAAACTAAAAAGCGCCTGGGCC |
| | | TTCAAGACTCTCTAAAGAGTTAGAGTCCTAAGAAAAGTCCGACGAGGTAGATGTTGGGGTGTCAACGCCGCTGGGGGCAG |
| | | CATGGTGAGCAAGATTGACATATTCCATATCTTAATAGTACCATCGTCTGAACCAGATGCTACACGAGTCGAGTCGTGCGAC |
| | | AAGGCCACCGAAGAAACCTTCGTACTGTGACCCTCAAGCACCCGTAGGCATTGC |
| SEQ | 37 | TTATGGGGTGGTATCGCATGTTATCTTCGTCCCATTGCAACGACCTACATACATACTCCTTTTCCGGTATTACCCCCACTATG |
| ID | | CATTGATGGCGTTACTAAGCTTTCTGGTTGCGTTAGCTTGATAAAACATTCCCACGTACGAAGCTACCTCGAGATATGTGAA |
| NO | | GCTGAGTGGTGCTCTATTAGTCGAGGGAAGATGAAATCATCCAGTCATATATCATCATAGGTATGAAGCGCCCATACGGGTA |
| | | AGGTGCCGCTACTACGCGAGCAAGTGGCTGCAGCAATGCCTTGGGAGACGAATCGGAGTTGATGTTGACCCTCTCTTCCA |
| | | GTAAGTAATAAAGCAAATCAGGAAGCTACCTACGCTTCACCCACGTACCTTATGCAAAGCAACTGATCTGGGTTAAAGAAAG |
| | | GCTTATACATAGCTCAGCGCTCCGGTAGTGGATGAGCTTGCTACCAATTGCGTGAAAATTGCGAAAGAGCATGTCTCGATC |
| | | CCAATGCTTGATGAATGACTTAGCCGTACGTGTAAGAGCTTTATAGAAAGTAGATAGCCCCGTAACCTGTGTACACATCGAG |
| | | CAGCGTCCCGCAACCAGGTCATGTTGAAGATGTTACATATCTCTTGAACCAGGTAGAAGTGACATGTCAGCTACGAGACCT |
| | | GGTGACTATTGAGAAAACGCCAACAAACGCAAGCGCTGAGTTTGCTCAACAACGTGAGACCAAAGTTAGTTTACGTAGTTTA |
| | | CGTAGCTCACAAGGCTCATTCAGCCTCTAGTCCGATATTCCTAGACGTCTGCAAAGCTTCAGCTACTGAGGACACCATTGGC |
| | | TGTCTTCTTCTCGGAGATGTTCTTCGCAATAGGCAGGTCGCAGAGCAAGACATGCTCAACCCTCTCAACAGTTATTCCTTTTT |
| | | CTCAATGGTGCCTTCCTTCTGGTTCGACCGGCATCTAATGACGTGTAGATATGGCTAAGATATCTTCAAGCTTATGCGAAAA |
| | | GTGTCTCCCGCTTCCCAGGAAAATCCTACATTCCTATTTCGATCACCACCTCCGATTGCATCTTCAAGCCCTTGTTTTCAGCA |
| | | ATGAGAACCATTACAAGACGAACCGGAGCAGTGTACACCCATAGAATTGTAAGTATATAAAGACCCTCTCGGAACCTCCTTC |
| | | AACTCATACCTATACAGGAGCAACAGCTGTTCGCCCCACAGTCCACGATCGACCAACATCTTCATCCAAAGGCTTTGCCCGT |
| | | GTCAACTGTCTCACTTCCACCCATCTCAACTGCACATCTTCATCCGAAACCATCAAGATGAAGACCCTCGCTAATAAACTGTT |
| | | CGTCGATGAGGGAGATACTGAGGAGCGCCTGCTCAGATCTAGAATTCATCGGGAGCAACAACATGAGGTTCGAACTAGAAT |
| | | ATGAGACCAAGTGCACGCGACCATAGCAATGAAGTACAACACGAAATATATGTACTTGAGCACTGAAGCATTACAATAGAAA |
| | | ATAAGTGATCTGTCATTGTTGAATATCTTATATACTATGTGCTGTCAGACGGAGCATTTGACTGCAAAATTGTTGGTTTTGCAT |
| | | TGGGCATTTTTCCATGGCAATAGCAATAGCTAAAACTCAGCCTCCCACGTCTCTGCTACTTATACTCATACAAAGCTATTTAT |
| | | TGTATAGCAACGCTGTATCAAGAAGCATGTAGTACTAATAGGGTACAGTTTGTGTCATGTCATTATATCACTGAATTGGGAGA |
| | | TGGAGCAGATATACGCACCACAAAGGTCACTCATTTTTCAGTTTTCCAGGATTGATTGTCGTGCCTATCTATAAAACTTGTTT |
| | | CGATGGTATTCTTATTTTAGATGCAGCTTGACAACATACATAATACAGTGAATGACGGATTTACTACGGGAATCATGGCTCGG |
| | | AGCCTCACTGACTGATGACGAGGCTCTCACAGCTAGAGAAGGGTAGGTCATACGTCTCTGAATCACGATGAATATCCAGAA |
| | | ATAATCGCTTCCTATGAATTGCACAACTCCATAGGTAGAGGTGCTCATGGGGATTTGTTACTGAGCACGATATAAGCTGGAC |
| | | GACCACGTGCTAAAGCTCGGGCCATTCGTCGTTGATATAATTGCTCTGCGTATACCAGGAGCAGATTCTTCGCAGATTACTT |
| | | GATATAAGTACTGCTCTAGTTACGCCCACAAAGGCGAGAGATAAGCATACGGCAGAAGCTTGCAATCTACGACATCATTGAG |
| | | CTTGGAACAACGCAATGAGTAGAATTACTTTGACTCAAGTATTGGGCGGTATGGTAGGCATTGGATGTCGTAAGTTACACAT |
| | | ACTCTTTTACCAATGAAGACAGCGGGTACTAGCCTACATACTTTTGAGTGAGTCACAAATGAGGGTGTTGTATGAAGCATTC |
| | | GCCATCAAAAGCTGGACATGCGTTGTGGCTAATGAAGTAGAAAAGGGGGTCTGCTGGCGTGTCCTGTGGTTTGCTTGTTAT |
| | | GCGGGACTCAACAATGGACGCAACGTCAACTTATTCTATGAGCGAATAGCTCTGACATGATGCTCCAGTGCTTGAGGCAGA |
| | | CAAAGCAAGTCAGCAATGACAAGGGCTTATGATTGGTGAATGATACATCAGCCTTAATGCGC |
| SEQ | 38 | CCTGTTCATGCCCGGTCACCAGGCCGCTCTGATTGCTGGGACGGCGGATTTAACATAGACTAACGGATTAGACTTAATTCG |
| ID | | GCCGCCATGTTGTTGATCCCTAGTCGATTGGGCTAGTAGTTCTTCTTTGCTCAGTCCCCTATGCGAGCCAGGAGCACGAGG |
| NO | | CAGATCCGAGTCAAGCAAGGCATGGATGGGATGGGATGGCTCTCGTGGCTTTGAATTACCGTTGATGCGAGAGAGAGAGA |
| | | GAGATTGGGCCATACGAGGCCTGATTCGTGCTTGCGGCTGGTGACTGGCCGTCTGGGGGCCCGAGCATGCCACTGGCAG |
| | | TGGGATGCGGTGGAACACGATTGCAACATTGCCCCCGAAAGGATGGGCTGGTAACTTCGTCTGTTCAGGTCGACCTCGGT |
| | | CATACAAAGCAAAGACGCGGGCGCAGCGCCATTGCCCAAGTCATGTCGGCCCGCCTACCCCAGCCTGTCATATCGTCACA |
| | | CGGACTGAACTGTGGGCTGCTGTTTAAGCGTTGAGCAGGAAGACCCTTGATAGACAAAGATCGGCAATGGATATCTCTCTC |
| | | GAGGCCTGCTTATTTCCCTGGCCTCCGCAGTGTCGACATTGCACGGCCAGCCACGACTCGGGATTGGCCGGCCATAGGTA |
| | | GTGGCGAGGACGAATGGTCAAAGGACTATGTATCATCTCGACTTATGCGCCTCGGGAGACAAGTCACACTGTTGCGCCGTC |
| | | TCCGAGCGAATGGCCGTGGCCATGGGCTAGCAGGACTCTGGACTTCTATCTTGGGATGCTTCACGATACGTGATCGACAAA |
| | | GGTTCCAGCGCCTCCATTGGAGCAAGGCAGAAATGGCAGTACGCTCCTCGGCCGACCTCTCTTGGTGGGTTAGGAGACCA |
| | | GGCACGCCCTCCACATGGATGCCACCCCTGGACGCGGACGCTTCATTCGCTCGCGGGCCGGTAACGACGATCTACAGCCA |
| | | TCTGGCCTGATTGCGGAGGTCAATTCCACCATCTCGACTCCTCCTCCCATCACCATGCCCCAGCAAGTGTGTGAAGCTCGG |
| | | GGAGGATGGCGCAAACCGACCTTCCTAAACCATGCTCTTGAGGCCGTCAACAACGACAATGCCCCGGAGATGGCAGATTAT |
| | | CGACGAAGCGTTTGTCGATGGAGCTCAAGGGGATATATAAAGACGTCCGGCGTCCCTCCGTCTGCTGTTGGAGCTAATAAG |
| | | TCTGGCTCTCAAGACTTGTAAATACTCTTGGTGTATATAGCAAGATGTCAGCACATAGCAGAGCAATGACGAAACTGTAATG |
| | | AAGGACGCTTTGGGTCTCAGTGACTTGTGAAATGAGGTCCCTTCCTACCTATCCATCGTGGTCCGTAGGGGTAAGGTGCGA |
| | | AGTCTACTTGCAATGCTTGAACTGAGCCTCGGAATGATACAATTATCCATGGCGATTGTGAATACTGAGATGAGGTCTCGTT |
| | | GATGATGTGCTTAGCAATATCAACATTAAATCCTGCTTTGCCCCATCCTGACCTTGATCTCCAAGCCACTGACAAATAGGTGT |
| | | TCATGCACTATCATGGGTTACTTGATGTATCGCTCCGGATATTTAGCTGCATCACTGTTAATGATTAAATCCAAGTCCTGTGT |
| | | TATACATTAAAGAAGGTCCAGAGTGGTGGTTCTGCCCGGCTTCGGAGATTTGCAAGGTCTCGAGCGAGTCCGAAGCTGGG |
| | | CTGAAGCAGCGCAGGGCCTGACTCTGGGTATGTGTTGATGTAGTGGCGGACGATGCCATGAACATGGACTTTTTCTTTTCT |
| | | CTTACCCCTGTGATGCTCTTTGTCATGGCGAGAACGTATGCTGAGAACAGTTGATATGAGTGAGATTGTTTCGAGGAGGGA |
| | | CATCAAGGGCTTGATGTCTTTGGTCACCAGCCTTTCTGCTTCTAACTCGGAATCATGAAAAAACCGACGTCTTGCTGACAGA |
| | | TATCGCCCTGGGGCCTAGAATATACAGAAGGATAGTTCTGACTGTGACAGCCAGAGAAGTGGTAGGGCTGTAAGGGGGTA |
| | | CGAGATGAGCTGTGCTCGATAGGTGAGCTGGAATGCTATTTGATGGATGTACAGCGAGTGTTGGGGTGGTTTTGCAGCTGT |
| | | AAATAAGGGAATTGACAGGGGAGGCGATGAACACGCCGGCAAGGAAAGCGTCTCATGCTATGCATGGGGATTTTGAACAA |
| | | GAAGAGTGCAGAATTTACTCCTGTGAAGAAGGGGAGTGATGAGAAGCGATATAGGGGATGATTGACGAGGGAGTTGATGAT |
| | | TGAGAGGGTTCTTACATAGTGCGGCTCTTTGATGACTTTGCAGAGTGTCTATTTAGATTGTGTTATTTATGGTTTCTGCTGCG |
| | | TTTGTGATTAGTTGATGCATGTATAGCGTCAAAAGCTCTTTTTACGCTGCCATGCCATC |
| SEQ | 39 | GTGAGCATATCAGCTGGTACTACTTGAAATATCACCTCTATCAGGAGCATACGATACAAGACTTCTACTATACAATCACCTGA |
| ID | | GAGGCTTCCAGTCGCAAGCGCCAGTCAATCTGATTGTAATATGGCGGACTAGACACGAGTATGCAAGCAGAAGCATCTTCC |
| NO | | TCGTTCGTCTTGATCGTCACTATAAGTCGCAATATACAGTCTAATTCTAGAGGCCAGTGTGACCTCATGAGGGACTATGGGA |
| | | AAACAAGGAAGAGATACCGCGACATGACGGGAGGTACTTGGCGGACTTGAGGCAAATAACATGCGCGCCAGTAGTGACAT |
| | | CACAAGCCCAGGCGATTGCCTCTGACTTATGGAAGATGATAGAAAGAAAGAGACCAGCCTCGTCTTTTCCTCACCAAGAAG |
| | | ACAAGGCAGTGTGAAGTAATCCCACTCGAGCAGGTAACTACGATGTCTGCCGCTTTCCCTCTCAGAACGTGGAGAAATCGG |
| | | AATATCGGCATCGAACCTAGTAGATGCCTGAGCAAACTAGCCGGGGTCGGTACTTTGTAAGAGCAAATCAGGAAGGTGATT |
| | | AGTCCCCGATAGTTACTGAGATCAGGCAAGATCGCCAAGGCGCGAATCCGATGAAGAATGCTTAAACATTAGGTTGTCTCT |
| | | CCCCCAGTCCGGTAATAACCATAATGATCCGCAATACCATTGTGTGTGTTGCGAGATTCTGCTGTAACTCGTGAATCAAAGA |
| | | ATCTCCAGACTCCTCATATCCGTAGCTTCTCATTCTTTATCTCGTAGTACCTCTAGCCAGGAACCAGCAGTATCACTGCACTG |
| | | AAGTTGCACGGCATCTCGGCACGTCGATGCGGCCAGCAATTCCTCCGGTGCTACTCCCCACCCACGTCTGGTCCGTAGGG |
| | | TGTGGTAGCATGTATCGCCCAATTTAGTTTGTCTGCTGCGGGAAGGACTTATGTTGCAGCGAATCCCATGTGAAGCATTGCA |
| | | TTGGCCTCCTCACAGTGGCGTAGGCGCCATGTTATCACGAACCTCAACACGCGAATACGGCTTAATGAGGAATCTGGATGT |
| | | TGACGCCAGCTCGGAGATGTCATTCAAGCAGGAAGGTGTCTTGAGAAAGTCTGCAAATGGCTCGTGGACCTAAGATCATTG |
| | | GCATCTTAAGATGTGAGTAGTTGGGGAAGGGATCTCGATTGCTCTCAAGTACTTCAGTTATAAATATTACCCTCTGCCTCTTC |
| | | TTGAGTTGATCTTGGTTTAAATCAGACTTAATAACAGGACCTTTGGCCGCTCTATCTGGCAGTTCTCCCGGCAACATGTAGC |
| | | AGAAGTATCATGTGCCACAAATTTTTGAGTCTATACATACTGATGATACAAATGAATACGAAACTTTTCATTATCAAATCCTCA |
| | | AGTATTAAAGTAATGTGACTGGCGATGAAGAGAGGACGTGGCTGTTGGCAAGGGCGTGTGCAATATTATATTGTACAGAATA |
| | | CATGTCGCAAAGCTATACAGGCGTTCTATTACCATTAGGACGTTGTCTGAAGAAAGTATACAGGCCTCCTTCTGCCATACCT |
| | | GCAATCTGCATAATAATAGGATGCAGAAGATATAAGATCATAGCGTAAAGGGGTGCTATCAAATGTCCATTTCAAATGTAACA |
| | | ATTCAGCCTCCCCGAGGGACGCCACTGGCCTTGCGGGCAGTGAAAATTCTGAATGAGAAATACTCACGGCGACTGTTGCTC |
| | | GGCAAAACCTTTACTGTGATTCCATTCATTATTCGTAAGTTTACTATGTGGCTTGCATAAGCCACAGTGATGGCGACATAAGC |
| | | ACAACTGCTCTAAACCAATAGCTCATAACATATTCTAGCATGACTGACTGACTACTTTGAGCCAAGCTGATGTTGTACTGCCT |
| | | TCTACTCAACTTGGCTCGCTTTGAATCAAACATTGCTTACGCGGGGTATATGAAATTCGCATCTTTCTTCCGTAGGAGCCGAT |
| | | AAAATTCCCATCCCCAACAAATTGAAAGCTGGTATGCCATTTCATATCGCAGTGGCCCCTTTACTTAAGAGCGACTATAATGT |
| | | GTGCCAAGTTTGAGAGTGTGAAATTGGAGCAGCAACTTATGGTATATTAACCCATGTTAGGAGCGTGAGGGGTTAGAAAGTA |
| | | CTATTGGCACAACATGGCATGGAATAATGGCGATGGTTTGTATAACTCCTGATAAAAACTCAGCTTTGGAGTGAAGCGCTCC |
| | | GTATGTTAGCCAGGGCTGAGACATTCTCTACAGAGAGACCTGCTCTTGCCTTGCTGTTAATCTTTCCTTCTATTTCTTTTCAT |
| | | ACAAATGTGCTGGGATGGCACATCTCACAATAAATTCAAATGGTGTATTTTCTGGCAGGCAGTCTGCTTCCCCAGAACCGGG |
| | | GCGGCAATGGCTATATCAAATGTAGAGTCCATACATGGGGCGCCTGCGCTCAATCACAACATAGCTGTCGAAGTGTCAAAC |
| | | AAAATCTCACCAGCCAGCTCCTTGGGCCTTCG |
| SEQ | 40 | AAATATTACCCTCTGCCTCTTCTTGAGT |
| ID | | |
| NO | | |
| SEQ | 41 | CTGCTACATGTTGCCGGG |
| ID | | |
| NO | | |
| SEQ | 42 | TGTGAGTAGTTGGGGAAGGGAT |
| ID | | |
| NO | | |
| SEQ | 43 | TTTGTATCATCAGTATGTATAGACTCAAAAATTTGTGG |
| ID | | |
| NO | | |
| SEQ | 44 | CTGGATGTTGACGCCAGC |
| ID | | |
| NO | | |
| SEQ | 45 | TAATATTGCACACGCCCTTGCC |
| ID | | |
| NO | | |
| SEQ | 46 | TAGCATGTATCGCCCAATTTAGTTTGT |
| ID | | |
| NO | | |
| SEQ | 47 | ACCCCTTTACGCTATGATCTTATATCTTCT |
| ID | | |
| NO | | |
| SEQ | 48 | GCAAATCAGGAAGGTGATTAGTCCCC |
| ID | | |
| NO | | |
| SEQ | 49 | TACGGAAGAAAGATGCGAATTTCATATACCC |
| ID | | |
| NO | | |
| SEQ | 50 | AGAACAGCAGCAGCGCT |
| ID | | |
| NO | | |
| SEQ | 51 | CGCCATTAGATGTTTTATACCTGCTTACA |
| ID | | |
| NO | | |
| SEQ | 52 | ACTGGGCAGCCTCCC |
| ID | | |
| NO | | |
| SEQ | 53 | CTTCCTGCAATGAGCAGCTCA |
| ID | | |
| NO | | |
| SEQ | 54 | CAGCGAAGATGGTACTCCCCT |
| ID | | |
| NO | | |
| SEQ | 55 | GCGATGGATTTTGGAGACGGT |
| ID | | |
| NO | | |
| SEQ | 56 | CTAGACATGGGTCGAAAGTGGC |
| ID | | |
| NO | | |
| SEQ | 57 | CAAGATCGGTGGGGGTGC |
| ID | | |
| NO | | |
| SEQ | 58 | AACGTTCCGCATCGGCC |
| ID | | |
| NO | | |
| SEQ | 59 | CGAGGGGGCAGAGTTGC |
| ID | | |
| NO | | |

Statements (features) and embodiments of the methods and compositions as disclosed herein are set out below. Each of the statements and embodiments as disclosed by the invention so defined may be combined with any other statement and/or embodiment unless clearly indicated to the contrary. In particular, any feature indicated as being preferred or advantageous may be combined with any other feature or features indicated as being preferred or advantageous.

### Embodiments

The present invention provides at least the following numbered statements/embodiments:
1. A method for genome editing within a cell comprising,
   a. contacting the cell with at least one pair of ribonucleoproteins such that the at least one pair of ribonucleoproteins are introduced into the cell
      i. whereby the ribonucleoproteins are pre-assembled in vitro, and
      ii. whereby each pair of ribonucleoproteins targets one locus in the cell; and
   b. further contacting the cell with at least one donor-DNA construct such that the at least one donor-DNA construct are introduced into the cell
      i. wherein the at least one donor-DNA construct has a 5′-end sequence which is at least partially complementary with the genome of the cell upstream of a first break in the genome of the cell,
         where the first break is caused by the first of the pair of ribonucleoproteins, and
      ii. wherein the at least one donor-DNA construct has a 3′-end sequence which is at least partially complementary with the genome of the cell downstream of a second break in the genome of the cell,
         where the second break is caused by the second of the pair of ribonucleoproteins, and
      iii. wherein the at least one donor-DNA construct serves as a template for the repair of the first and the second break by homologous recombination repair; and
   c. further contacting the cell with a selectable marker such that the selectable marker is introduced into the cell; and
   d. optionally, screening the cell for the genome edits introduced by the donor-DNA construct.
2. The method of embodiment 1, where the cell is contacted with at least two pairs of ribonucleoproteins and where the cell is further contacted with at least two donor-DNA constructs such that said ribonucleoproteins and donor-DNA are introduced into the cell.
3. The method of embodiment 1, where the cell is contacted with at least three pairs of ribonucleoproteins and where the cell is further contacted with at least three donor-DNA constructs such that said ribonucleoproteins and donor-DNA are introduced into the cell.
4. The method of embodiment 1, where the cell is contacted with at least four, five, six or more pairs of ribonucleoproteins and where the cell is further contacted with at least four, five, six or more donor-DNA constructs such that said ribonucleoproteins and donor-DNA are introduced into the cell.
5. The method of any one of embodiments 1 to 4, wherein the selectable marker is contained in a self-replicating episomal plasmid.
6. The method of embodiment 5, where the self-replication episomal plasmid is an AMA1-based plasmid.
7. The method of any one of embodiments 1 to 6, where the 5′-end and 3′-end sequences of the at least one donor-DNA construct flank an additional nucleotide sequence which is inserted into the genome of the cell, at the targeted locus, by the homologous recombination repair.
8. The method of embodiment 7, where the additional nucleotide sequence comprises a nucleotide sequence encoding a protein of interest.
9. The method of embodiment 7, where the additional nucleotide sequence comprises a nucleotide sequence capable of expressing a protein of interest.
10. The method of embodiment 8 or embodiment 9, where the protein of interest comprises a VHH.
11. The method of embodiment 7, where the additional nucleotide sequence does not comprise a selectable marker.
12. The method of any one of the preceding embodiment, where the ribonucleoprotein comprises a Cas endonuclease.
13. The method of embodiment 12, where the Cas endonuclease is selected from the group consisting of Cas9, Cas12a, Cas12e, Cas12f, Cas12i and Cas12j endonucleases.
14. The method of embodiment 12, where the Cas endonuclease is a Cas9 endonuclease
15. The method of embodiment 12, where the Cas endonuclease is a Cas12a endonuclease.
16. The method of embodiment 12, where the Cas endonuclease is a Cas12e endonuclease.
17. The method of embodiment 12, where the Cas endonuclease is a Cas12f endonuclease.
18. The method of embodiment 12, where the Cas endonuclease is a Cas12i endonuclease.
19. The method of embodiment 12, where the Cas endonuclease is a Cas12j endonuclease
20. The method of embodiment 12, wherein the Cas endonuclease is selected from the group consisting of Cas9, Cas12a and Mad7.
21. The method of any one of the preceding embodiments, where the cell is a filamentous fungal cell.
22. The method of any one of the preceding embodiments, where the cell is a Trichoderma species.
23. The method of any one of the preceding embodiments, where the cell is a Myceliophthora species.
24. A composition comprising
   a. an RNA-guided DNA endonuclease and at least one pair of guide-RNAs
      i. whereby the endonuclease and the at least one pair of guide-RNAs are capable of assembling in vitro into at least one pair of ribonucleoproteins, and
      ii. whereby each pair of ribonucleoproteins targets one locus in a cell; and
   b. at least one donor-DNA construct
      i. wherein the at least one donor-DNA construct has a 5′-end sequence which is at least partially complementary with the genome of the cell upstream of the target sequence of the first of the pair of ribonucleoproteins, and
      ii. wherein the at least one donor-DNA construct has a 3′-end sequence which is at least partially complementary with the genome of the cell downstream of the target sequence of the second of the pair of ribonucleoproteins, and
      iii. wherein the at least one donor-DNA construct serves as a template for homologous recombination repair of a first and a second break in the genome of the cell, wherein the first and second breaks are caused by the first and second of the pair of ribonucleoproteins, respectively; and
   c. a selectable marker.
25. The composition of embodiment 24, where the composition comprises at least two pairs of guide RNAs capable of assembling in vitro into at least two pairs of ribonucleoproteins and containing at least two donor-DNA constructs.
26. The composition of embodiment 24, where the composition comprises at least three pairs of guide RNAs capable of assembling in vitro into at least three pairs of ribonucleoproteins and containing at least three donor-DNA constructs.
27. The composition of embodiment 24, where the composition comprises at least four, five, six or more of guide RNAs capable of assembling in vitro into at least four, five, six or more pairs of ribonucleoproteins and containing at least four, five, six or more donor-DNA constructs.
28. The composition of any one of embodiments 24 to 27, wherein the selectable marker is contained in a self-replicating episomal plasmid.
29. The composition of embodiment 28, where the self-replication episomal plasmid is a AMA1-based plasmid.
30. The composition of any one of embodiments 24 to 29, where the 5′-end and 3′-end sequences of the at least one donor-DNA construct flank an additional nucleotide sequence which is inserted into the genome of the cell, at the targeted locus, by the homologous recombination repair.
31. The composition of embodiment 30, where the additional nucleotide sequence comprises a nucleotide sequence encoding a protein of interest.
32. The composition of embodiment 30, where the additional nucleotide sequence comprises a nucleotide sequence capable of expressing a protein of interest.
33. The composition of embodiment 31 or embodiment 32, where the protein of interest comprises a VHH.
34. The composition of embodiment 30 where the additional nucleotide sequence does not comprise a selectable marker.
35. The composition of any one of embodiments 24 to 34, wherein the RNA-guided DNA endonuclease is a Cas endonuclease.
36. The composition of embodiment 35, where the Cas endonuclease is selected from the group consisting of Cas9, Cas12a, Cas12e, Cas12f, Cas12i and Cas12j endonucleases.
37. The composition of embodiment 36, where the Cas endonuclease is a Cas9 endonuclease
38. The composition of embodiment 36, where the Cas endonuclease is a Cas12a endonuclease.
39. The composition of embodiment 36, where the Cas endonuclease is a Cas12e endonuclease.
40. The composition of embodiment 36, where the Cas endonuclease is a Cas12f endonuclease.
41. The composition of embodiment 36, where the Cas endonuclease is a Cas12i endonuclease.
42. The composition of embodiment 36, where the Cas endonuclease is a Cas12j endonuclease
43. The composition of embodiments 35, where the Cas endonuclease is selected from the group consisting of Cas9, Cas12a and Mad7.
44. A cell obtainable by the method according to any one of embodiments 1 to 16.
45. A method for the production of a protein of interest comprising
   a. providing a cell obtained from the method of any one of embodiments 8 to 10 capable of expressing the protein of interest, and
   b. cultivating the cell under conditions suitable for expressing the protein of interest, and
   c. optionally isolating the protein of interest.
46. A high-throughput method for transforming fungal cells comprising the steps of:
   a. providing fungal protoplasts, and
   b. mixing the protoplasts with a nucleic acid construct, and
   c. further mixing the protoplasts with a polyethylene glycol solution
   d. incubating the protoplasts and subsequently adding an additional solution of polyethylene glycol to the protoplasts mixture
   e. resuspending the protoplasts and plating and selecting the antibiotic resistant colonies
   f. optionally screening the cell for the presence of genome edits introduced by the nucleic acid construct of step b.
47. The high-throughput method of embodiment 46, where between 5 to 200 μl of protoplasts are used.
48. The high-throughput method of embodiment 47 where between 5 to 50 μl of protoplasts are used.
49. The high-throughput method of any of the embodiments 46 to 48 where the concentration of protoplast is lower than 1.10\hat{ }8 cells/ml.
50. The high-throughput method of embodiment 49 where the concentration of protoplast is between 1.10\hat{ }2 and 1.10\hat{ }5 cells/ml.
51. The high-throughput method of any of embodiments 46 to 50, where the polyethylene glycol has a molecular weight between 1000 and 8000 Da.
52. The high-throughput method of embodiment 51, where the polyethylene glycol molecular weight is between 4000 and 6000 Da.
53. The high-throughput method of embodiment 51, where the polyethylene glycol molecular weight is 4000 Da or 6000 Da.
54. The high-throughput method of any of embodiments 46 to 53 where the incubation time is less than 30 minutes.
55. The high-throughput method of embodiment 54 where the incubation time is between 5 and 10 minutes.
56. The high-throughput method of any of embodiments 46 to 55, where the volume of polyethylene glycol solution in step c is lower than 50 μl.
57. The high-throughput method of embodiment 56, where the volume of polyethylene glycol solution in step c is between 5 and 20 μl.
58. The high-throughput method of any of embodiments 46 to 57, where the volume of polyethylene glycol solution in step d is lower than 2000 μl.
59. The high-throughput method of embodiment 58, where the volume of polyethylene glycol solution in step b is between 100 and 500 μl.
60. The high-throughput method of any of embodiments 46 to 59, where the protoplasts in step b are mixed with the composition according to any of the embodiments 22-40.
61. The high-throughput method of embodiment 60, where the total volume of the composition according to embodiments 17-29 is lower than 25 μl.
62. The high-throughput method of embodiment 61, where the total volume of the composition according to embodiments 17-29 is between 5 and 15 μl.
63. The high-throughput method of any of embodiments 46 to 62, where the fungal protoplasts are from Trichoderma species or Myceliophthora species.

1. A method for genome editing within a cell comprising,
a. contacting the cell with at least one pair of ribonucleoproteins such that the at least one pair of ribonucleoproteins are introduced into the cell
i. whereby the ribonucleoproteins are pre-assembled in vitro, and
ii. whereby each pair of ribonucleoproteins targets one locus in the cell; and
b. further contacting the cell with at least one donor-DNA construct such that the at least one donor-DNA construct are introduced into the cell
i. wherein the at least one donor-DNA construct has a 5′-end sequence which is at least partially complementary with the genome of the cell upstream of a first break in the genome of the cell,
where the first break is caused by the first of the pair of ribonucleoproteins, and
ii. wherein the at least one donor-DNA construct has a 3′-end sequence which is at least partially complementary with the genome of the cell downstream of a second break in the genome of the cell,
where the second break is caused by the second of the pair of ribonucleoproteins, and
iii. wherein the at least one donor-DNA construct serves as a template for the repair of the first and the second break by homologous recombination repair; and
c. further contacting the cell with a selectable marker such that the selectable marker is introduced into the cell; and
d. optionally, screening the cell for the genome edits introduced by the donor-DNA construct.

2. The method of claim 1, where the cell is contacted with at least two, at least three, at least four, at least five, at least six or more pairs of ribonucleoproteins and where the cell is further contacted with at least four, five, six or more donor-DNA constructs such that said ribonucleoproteins and donor-DNA are introduced into the cell.

3. The method of any one of the preceding claims, where the cell is a filamentous fungal cell.

4. The method of any one of the preceding claims, where the cell is a Trichoderma species or a Myceliophthora species.

5. A composition comprising
a. an RNA-guided DNA endonuclease and at least one pair of guide-RNAs
i. whereby the endonuclease and the at least one pair of guide-RNAs are capable of assembling in vitro into at least one pair of ribonucleoproteins, and
ii. whereby each pair of ribonucleoproteins targets one locus in a cell; and
b. at least one donor-DNA construct
i. wherein the at least one donor-DNA construct has a 5′-end sequence which is at least partially complementary with the genome of the cell upstream of the target sequence of the first of the pair of ribonucleoproteins, and
ii. wherein the at least one donor-DNA construct has a 3′-end sequence which is at least partially complementary with the genome of the cell downstream of the target sequence of the second of the pair of ribonucleoproteins, and
iii. wherein the at least one donor-DNA construct serves as a template for homologous recombination repair of a first and a second break in the genome of the cell, wherein the first and second breaks are caused by the first and second of the pair of ribonucleoproteins, respectively; and
c. a selectable marker.

6. The composition of claim 5, where the composition comprises at least two, at least three, at least four, at least five, at least six or more of guide RNAs capable of assembling in vitro into at least four, five, six or more pairs of ribonucleoproteins and containing at least four, five, six or more donor-DNA constructs.

7. The method of any one of claims 1 to 4, or the composition of any one of claims 5 to 6, wherein the selectable marker is contained in a self-replicating episomal plasmid, optionally wherein the self-replication episomal plasmid is a AMA1-based plasmid

8. The method of any one of claims 1 to 4, or the composition of any one of claims 5 to 7, where the 5′-end and 3′-end sequences of the at least one donor-DNA construct flank an additional nucleotide sequence which is inserted into the genome of the cell, at the targeted locus, by the homologous recombination repair, optionally wherein the additional nucleotide sequence comprises a nucleotide sequence encoding a protein of interest or a nucleotide sequence capable of expressing a protein of interest, such as a VHH.

9. The method or composition of claim 8 where the additional nucleotide sequence does not comprise a selectable marker.

10. The method of any one of claims 1 to 4 or 7 to 9, or the composition of any one of claims 5 to 9, wherein the RNA-guided DNA endonuclease is a Cas endonuclease, optionally selected from the group consisting of Cas9, Cas12a, Cas12e, Cas12f, Cas12i and Cas12j.

11. A cell obtainable by the method according to any one of claims 1 to 4 or 4 to 10.

12. A method for the production of a protein of interest comprising
a. providing a cell obtained from the method of any one of claims 8 to 10 capable of expressing the protein of interest, and
b. cultivating the cell under conditions suitable for expressing the protein of interest, and
c. optionally isolating the protein of interest.

13. A high-throughput method for transforming fungal cells comprising the steps of:
a. providing fungal protoplasts, and
b. mixing the protoplasts with a nucleic acid construct, and
c. further mixing the protoplasts with a polyethylene glycol solution
d. incubating the protoplasts and subsequently adding an additional solution of polyethylene glycol to the protoplasts mixture
e. resuspending the protoplasts and plating and selecting the antibiotic resistant colonies
f. optionally screening the cell for the presence of genome edits introduced by the nucleic acid construct of step b.

14. The high-throughput method of claim 13, where:
a. between 5 to 200 μl of protoplasts are used;
b. the concentration of protoplast is between 1.10\hat{ }2 and 1.10\hat{ }5 cells/ml
c. the polyethylene glycol has a molecular weight between 1000 and 8000 Da;
d. where the incubation time is less than 30 minutes;
e. the volume of polyethylene glycol solution in step c is lower than 50 μl;
f. the volume of polyethylene glycol solution in step d is lower than 2000 μl;
g. the volume of polyethylene glycol solution in step b is between 100 and 500 μl; and/or
h. the fungal protoplasts are from Trichoderma species or Myceliophthora species

15. The high-throughput method of any of claims 13 to 14, where the protoplasts in step b are mixed with the composition according to any of the claims 5 to 10, optionally wherein the total volume of the composition according to claims 5 to 10 is lower than 25 μl.


SEQUENCE LISTING (59 sequences)

SEQ ID NO: 1 - protein, 119 aa, synthetic construct
  REGION 1..119: note = Synthetic VHH sequence.
DVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 2 - protein, 119 aa, synthetic construct
  REGION 1..119: note = Synthetic VHH sequence.
QVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 3 - protein, 10 aa, synthetic construct
  REGION 1..10: note = Synthetic VHH sequence.
RSIFSINAMD

SEQ ID NO: 4 - protein, 9 aa, synthetic construct
  REGION 1..9: note = Synthetic VHH sequence.
GITRGGTTK

SEQ ID NO: 5 - protein, 11 aa, synthetic construct
  REGION 1..11: note = Synthetic VHH sequence.
LRGEQPWTRDY

SEQ ID NO: 6 - protein, 117 aa, synthetic construct
  REGION 1..117: note = Synthetic VHH sequence
QVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 7 - protein, 10 aa, synthetic construct
  REGION 1..10: note = Synthetic VHH sequence
GTIFRPTAMG

SEQ ID NO: 8 - protein, 9 aa, synthetic construct
  REGION 1..9: note = Synthetic VHH sequence.
TITTGGSTK

SEQ ID NO: 9 - protein, 9 aa, synthetic construct
  REGION 1..9: note = Synthetic VHH sequence.
QWGVRTRDY

SEQ ID NO: 10 - protein, 126 aa, synthetic construct
  REGION 1..126: note = Synthetic VHH sequence.
QVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 11 - protein, 10 aa, synthetic construct
  REGION 1..10: note = Synthetic VHH sequence.
ISDRAFSRHV

SEQ ID NO: 12 - protein, 10 aa, synthetic construct
  REGION 1..10: note = Synthetic VHH sequence.
AIGWTGRRTY

SEQ ID NO: 13 - protein, 15 aa, synthetic construct
  REGION 1..15: note = Synthetic VHH sequence.
SHFYSVSFEINDYDY

SEQ ID NO: 14 - protein, 117 aa, synthetic construct
  REGION 1..117: note = Synthetic VHH sequence.
DVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 15 - protein, 126 aa, synthetic construct
  REGION 1..126: note = Synthetic VHH sequence.
DVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 16 - DNA, 9899 bp, synthetic construct
  misc_feature 1..9899: note = Synthetic plasmid sequence.
  source 1..9899: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagcttggtacccatgacacatgcatccaccatcgcagacttatcatcactgagcctccacctagcctgcggccgccttgacgaccgttgatctgcttgatctcgtctcccgaaaatgaaaatagctctgctaagctattcttctcttcgccggagcctgaaggcgttactaggttgcagtcaatgcattaatgcattgcagatgagctgtatctggaagaggtaaacccgaaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatccctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcgattgtgatgtgatggagttgagatgggggtgaggagatggatgatgggaaaggaagatggactgaggatggaagaagagaagaagagagagagagaaagtcttccaggagagaaagggaaccgaagaaaaatggggaggaaaccggccctagcacctaaatacgtctcccgcttaattttcgcccttttttcaccaaaccctccgcgtctttcgtgcgctagctgtcttggggggtgtgtaaaacttgggaacaaccctacgccgaacctcccgtacgaagcccgtacagtgtattctatccctggctttcccagcctggtaagtcgggtcccttcgggacggggccaaggagactgagtttccgggttaaccaataatgccgccagccgtggagcggtctgagctgtctatcgtgaatccgtgacgctgaattgctcagtccaagtcggagacgctggaatccaccggttgctccagccacggcgaagaatccacttaccttccgggcttccgccagctggcaacatatttttgaggctgatatagccttctcctccatcacgataagccctgactaccttgctacgggccaattgcaatttgtttgctggttacgctttacaaaaggtggccgttactgagcaaaaagaaaatgatgtaaaaatttgcgagtgggtcccatagctggatgggtccgataaaatggtactgccccacttagtggcagctcgcgaccagtcacaagcccaggaccgcaagctagaatgatagacggcacctcggacgcaaatcttgaccaatccggcccgaatcgggaatgcggctccacagctgcaggtttaaactagcagaagagcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - DNA, 9250 bp, synthetic construct
  misc_feature 1..9250: note = Synthetic plasmid sequence.
  source 1..9250: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagcttggtacctcatgacacatgcatccaccatcgcagacttatcatcactgagcctccacctagcctgcggccgccttgacgaccgttgatctgcttgatctcgtctcccgaaaatgaaaatagctctgctaagctattcttctcttcgccggagcctgaaggcgttactaggttgcagtcaatgcattaatgcattgcagatgagctgtatctggaagaggtaaacccgaaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcctttagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatatcgattgtgatgtgatggagttgagatgggggtgaggagatggatgatgggaaaggaagatggactgaggatggaagaagagaagaagagagagagagaaagtcttccaggagagaaagggaaccgaagaaaaatggggaggaaaccggccctagcacctaaatacgtctcccgcttaattttcgcccttttttcaccaaaccctccgcgtctttcgtgcgctagctgtcttggggggtgtgtaaaacttgggaacaaccctacgccgaacctcccgtacgaagcccgtacagtgtattctatccctggctttcccagcctggtaagtcgggtcccttcgggacggggccaaggagactgagtttccgggttaaccaataatgccgccagccgtggagcggtctgagctgtctatcgtgaatccgtgacgctgaattgctcagtccaagtcggagacgctggaatccaccggttgctccagccacggcgaagaatccacttaccttccgggcttccgccagctggcaacatatttttgaggctgatatagccttctcctccatcacgataagccctgactaccttgctacgggccaattgcaatttgtttgctggttacgctttacaaaaggtggccgttactgagcaaaaagaaaatgatgtaaaaatttgcgagtgggtcccatagctggatgggtccgataaaatggtactgccccacttagtggcagctcgcgaccagtcacaagcccaggaccgcaagctagaatgatagacggcacctcggacgcaaatcttgaccaatccggcccgaatcgggaatgcggctccacagctgcaggtttaaactagcagaagagcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 18 - DNA, 5249 bp, Aspergillus nidulans
  source 1..5249: mol_type = genomic DNA
agcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagct

SEQ ID NO: 19 - RNA, 27 nt, synthetic construct
  misc_feature 1..27: note = Synthetic guide RNA.
  source 1..27: mol_type = other RNA
tttcgcccgcatggattcgcgagacga

SEQ ID NO: 20 - RNA, 27 nt, synthetic construct
  misc_feature 1..27: note = Synthetic guide RNA.
  source 1..27: mol_type = other RNA
tttggaggacgccgtggtcggtcggga

SEQ ID NO: 21 - RNA, 27 nt, synthetic construct
  misc_feature 1..27: note = Synthetic guide RNA.
  source 1..27: mol_type = other RNA
tttgaggcacagaaacccaatagtcaa

SEQ ID NO: 22 - RNA, 27 nt, synthetic construct
  misc_feature 1..27: note = Synthetic guide RNA.
  source 1..27: mol_type = other RNA
tttcgccacggagctttacaggcactg

SEQ ID NO: 23 - RNA, 27 nt, synthetic construct
  misc_feature 1..27: note = Synthetic guide RNA.
  source 1..27: mol_type = other RNA
tttccataatccgccatcatgaagttt

SEQ ID NO: 24 - RNA, 27 nt, synthetic construct
  misc_feature 1..27: note = Synthetic guide RNA.
  source 1..27: mol_type = other RNA
tttgccaacaagaacaccaacgtctaa

SEQ ID NO: 25 - RNA, 27 nt, synthetic construct
  misc_feature 1..27: note = Synthetic guide RNA.
  source 1..27: mol_type = other RNA
tttccccccatgaagaactattgcatc

SEQ ID NO: 26 - RNA, 27 nt, synthetic construct
  misc_feature 1..27: note = Synthetic guide RNA.
  source 1..27: mol_type = other RNA
tttatgaggaaaagggaaaaaagggaa

SEQ ID NO: 27 - RNA, 27 nt, synthetic construct
  misc_feature 1..27: note = Synthetic guide RNA.
  source 1..27: mol_type = other RNA
tttcgccacaaacgtcctgctgctggc

SEQ ID NO: 28 - RNA, 27 nt, synthetic construct
  misc_feature 1..27: note = Synthetic guide RNA.
  source 1..27: mol_type = other RNA
tttgggtcttggtggagatcgtgccac

SEQ ID NO: 29 - RNA, 27 nt, synthetic construct
  misc_feature 1..27: note = Synthetic guide RNA.
  source 1..27: mol_type = other RNA
tttcttctcatcagccccatcagcctt

SEQ ID NO: 30 - RNA, 27 nt, synthetic construct
  misc_feature 1..27: note = Synthetic guide RNA.
  source 1..27: mol_type = other RNA
tttcacgttccatcattgatcccatag

SEQ ID NO: 31 - RNA, 27 nt, synthetic construct
  misc_feature 1..27: note = Synthetic guide RNA.
  source 1..27: mol_type = other RNA
tttcctaagctgcttgattgaaggatg

SEQ ID NO: 32 - RNA, 27 nt, synthetic construct
  misc_feature 1..27: note = Synthetic guide RNA.
  source 1..27: mol_type = other RNA
tttagagtatacagcttcggactcaac

SEQ ID NO: 33 - DNA, 3980 bp, synthetic construct
  misc_feature 1..3980: note = Synthetic donor-DNA.
  source 1..3980: mol_type = other DNA
ggcatagcaacgcaaagcctcagatcaaaacggcatcgctcccacccggacgctcagcctctgagactcttgacaagggtccgcaagacagcccgaagaacagacgcacgcgctcgatctgagcacacaccgaggccctcttttcggcgcccagtcctgggtaatacaattgtatctcgctgcctccaactcgacgcatacatacctacataccacctcactctacctacctcctccggatactactttgcaatacctcctcgtctgcgctggtcggctctttgacaaaccaagcctgccgattgctaaccataccaccctttacctactggagccctctgctacgagcacacagtttagagtaaccgtacgagtccgtacctccaacgctcaagctcgagagttgtgccttgcctgctctcttgactgaagccagaggacccgcccggccagagtgcggcggttcccccaatttcactcatccaagcaccggtcggcgccagctttccctctcttcctctctctctctctctgttcttttttttttctcccccattagccttcttactttcgcctgtcacaggcctacgacgtcaagcagagagaattgcaggggtactccgtgctgctcttactctttttctctcccttgatcccctctcgtcacccctccactcggactcgtatcatcctacgtacttaccagacagcattcggcgtgcacatcaaaggcgtagctttccctgtcattcatcccactttcatccagcaagcgtcgtccgaagggcttcccgcaatccatggcagctgtcggaccgcttgcgatgcacccgaccgtcacagaacacgattaccgcttcccgcgaagacccgatcctcatcgaagtaccgaccgcagctgcagctgtggagccgcattcccgattcgggccggattggtcaagatttgcgtccgaggtgccgtctatcattctagcttgcggtcctgggcttgtgactggtcgcgagctgccactaagtggggcagtaccattttatcggacccatccagctatgggacccactcgcaaatttttacatcattttctttttgctcagtaacggccaccttttgtaaagcgtaaccagcaaacaaattgcaattggcccgtagcaaggtagtcagggcttatcgtgatggaggagaaggctatatcagcctcaaaaatatgttgccagctggcggaagcccggaaggtaagtggattcttcgccgtggctggagcaaccggtggattccagcgtctccgacttggactgagcaattcagcgtcacggattcacgatagacagctcagaccgctccacggctggcggcattattggttaacccggaaactcagtctccttggccccgtcccgaagggacccgacttaccaggctgggaaagccagggatagaatacactgtacgggcttcgtacgggaggttcggcgtagggttgttcccaagttttacacaccccccaagacagctagcgcacgaaagacgcggagggtttggtgaaaaaagggcgaaaattaagcgggagacgtatttaggtgctagggccggtttcctccccatttttcttcggttccctttctctcctggaagactttctctctctctcttcttctcttcttccatcctcagtccatcttcctttcccatcatccatctcctcacccccatctcaactccatcacatcacaatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttcaggctccggcgaagagaagaatagcttagcagagctattttcattttcgggagacgagatcaagcagatcaacggtcgtcaaggcggccgcaggctaggtggaggctcagtgatgataagtctgcgatggtggatgcatgtgtcatgagacagcgtaggcatagcaagagcgagggcacttcatcgtcggctatggatctcgacggccctggagactctattgggcgacagcttggcgcaactcccagcatgccttccatctctagcactatgctttcgaatggaagcttcaccatgggtcagcgctccatgatgggatcaagcatggtgtccatgggtggttcccagggcaaccttgtgaacagcgccggtggctcgtcaggccctcaggaatgggagtggttgactatgagtctttgaagcggccggcgagatggcgaatttgacacgggatcgttgatgctagacgaaaagatacggcgtgcgatcagtgtttgggtcaaccttgaggaatggtggcaaggcttgagaaggggccgaccatggcctcacaatatgggctggctggcattgctgagatgagctgatggcggcgcgaaggatatgggcgctttaagtaatgggcatcttcatggcgtgttggttcaagaaatgatacctgggtcttttgtgtgtttttttggcttgcacgtacggactacctttcggaggttgacaatgtgttctatctttactcttttatttctttctgctctgcctgcgcttggatatgggcggcagtcttcgaagaacagcctcggtgcgataaccagcaactattttatacagacatggagagcgcccgtcaaggaactacctatttatgaatgccaggggcggtgtccaagggagccacgacgaccggtcccgcgcttacgacctgatgtcgacataatctggagtatgtt

SEQ ID NO: 34 - DNA, 4331 bp, synthetic construct
  misc_feature 1..4331: note = Synthetic donor-DNA.
  source 1..4331: mol_type = other DNA
acggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatcatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggatccgtggctgtggagaagcgcgacgtccagctcgtcgagagcggcggcggcctcgtccaggctggcggcagcctccgcctctcctgcgccgcctcgcgcagcatcttcagcatcaacgccatggactggtatcgacaggcccctggcaagcagcgcgagtgggtcgccggcatcacccgtggcggcaccacgaagtacgccgactccgtcaagggccgcttcaccatcagccgcgataacgccaagaagaaggtctatctccagatgaactcgctcaagcccgaggacaccgccgtctactactgcaacgtcctccgcggcgagcagccctggacgcgcgactactggggccagggcacccaggtcaccgtcagcagctaaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacaaacagcataatagcaacagtggaaattagtggcgcaataattgagaacacagtgagaccatagctggcggcctggaaagcactgttggagaccaacttgtccgttgcgaggccaacttgcattgctgtcaagacgatgacaacgtagccgaggaccgtcacaagggacgcaaagttgtcgcggatgaggtctccgtagatggcatagccggcaatccgagagtagcctctcaacaggtggccttttcgaaaccggtaaaccttgttcagacgtcctagccgcagctcaccgtaccagtatcgaggattgacggcagaatagcagtggctctccaggatttgactggacaaaatcttccagtattcccaggtcacagtgtctggcagaagtcccttctcgcgtgcgagtcgaaagtcgctatagtgcgcaatgagagcacagtaggagaataggaacccgcgagcacattgttcaatctccacatgaattggatgactgctgggcagaatgtgctgcctccaaaatcctgcgtccaacagatactctggcaggggcttcagatgaatgcctctgggcccccagataagatgcagctctggattctcggttacgatgatatcgcgagagagcacgagttggtgatggaggggacgaggaggcataggtcggccgcaggcccataaccagtcttgcacagcattgatcttcctcacgaggagctcctgat

SEQ ID NO: 35 - DNA, 2074 bp, synthetic construct
  misc_feature 1..2074: note = Synthetic donor-DNA.
  source 1..2074: mol_type = other DNA
ctcaaggactactactgccaatcttacctctatctctagtcatcttgttccaacgctttgatagagtaaacggtcagtagtagttgacatctctcgccttgactgaatctgccgagtcagacgcactgcaatcttacaacacctgagtcaccttattcttattttgactcagagcttaaagccgccagatgccggtccacagagctctctcttccgtacaaatgatcccaagcaaagcaaggagagcatcatcatattatccatgtgatccaacagagagagacaactttcgtgtacactcacctttaaaaaaaaacgccatgcatgagaaacgagaaatcaatgccgtcatccaaagtcaacctcatcgtcaggtgtgccaatggacgagcagagctgaaatgcagctgtgaccaagaagcagcgagcaagggggcagaaaggcacggccaacgcatatctgctgatggcctaatgtctcggtccatccctggggggaatcgtcaccaaggagaataagagttgtctcagaagaaagagagagagaaacaaaacagtgtctcagagaaatgccctcgtcttcccgcccccaggtgcggactccgcgcttttttgcttgtcctcaattgctgcaaaagacacgtcctgatgctcttgcttgcctcgaatcacattcctacacattcctggccaaggcttgggcagccatcacgcggatattcgacggctctatatggcaatgatccgtcagtaaacggaagatgggccttccgccatcgtgtagaatcgccatgatgcgggttggaatccaaaagtgcagggaggtcgtttcaggccggaatgccatgagtatataatacgcgaaagggtcgtgaataatccatctttctcccttccagtatcaaagggtttcttctctcccgtcttcttttgatataaaaggagcagagagccaccggaagctgatctcgattcgacattttctcttttttttctgccgtaataagattgagcatctagttagtagttggacattgtcttgggatgctgcttgcgactacctacctatgatacatgtatggaaggaagcagtcggttgcacggcttggatcagtaaacggtcggaatgtcaccagaagggggggaggaagggggtttcaaactggatgacagggtaacgtttcaatgttctctcatggggctaatgatggttgatgatgaaagaatctcttgtttcggcttttggaaagaatacgatagtattacctactagtactaggcagtcatcaattaagggggagatatcaatagggatgtcgatccgggttctggaatattcagtttcttcagatccatggctgcaaagagtttggcgactgtcaacatttattcaacctttttggtgccttgttgattaatctccgtgagatgcggtaatcgtcagctaaaaactatcattgcttgcatctcactaacacgactgggtcctcctcaccaaacggcacatgtagcttaaatccgttgcgactgctgcagcatcaagagtgccaaaaaaagccattgaatcgcttcgattgaaattctgttgaccagatggtactacagttacgacgaattcaaaagtctatgattcgtataagcgtatcgaattccgggtaagttggactggcttattcgtcaggcaccggctgccaagaaacggcaacctgcacgaagcagcaagaactgcccatggcctcggactttgctcatcaggccagtcatggaccgaggcagcctcgtcagatcatgccatctattggcctggacgagaaacctctgccacgtggagaccggcttttgagtcggcttggaggctgtcaaggacgcctcgagggctgcccaaggataagatatacaagagggacatccatatacatatatagggccaaatggacggcgaaccgggcgccgggcctcaccagctcaccgttgccaacctgcaaaccctcaaccagcaagtcaagcagttccacctcctccggcaccgcaaggcaatggcctccaacatcctcgtcctgggcgccggcgaact

SEQ ID NO: 36 - DNA, 2006 bp, synthetic construct
  misc_feature 1..2006: note = Synthetic donor-DNA.
  source 1..2006: mol_type = other DNA
gcataatatatcgaagccgccaccgaaacatcatagacaaacataaaagatgagcttcctacatcaaaccgacaaacatcatcttataatagatcatgatcgcagatgcagcattaatagcctctcgcaccagagcacaccaatcattcagccatcgtgaacaacaacccctgcctccttacaaagaatcctcagctctcaacgccaaaagtcctcctccaaccggcaactccgccgccagccaactgcttttctctcctcccatagactctcttttagatccatttctccagcaaaagaacaaaaagcctccggccggcaggggccgacatgcccctggctctgcaaatcatgccccatcaggaaggtcgcaaatagacgtgcaaagatgagggagctaatatgcatggttttgggcatgagctgcgggtcgcgggatcttttgaattgacttgacggacacgggcagcgattaacaatggaaaactcgagggaaggcaagaatcttctcctcttgtggacaacaaacggggtatgaacagagcccccccctcccctttgtttggggctgtcgtattataggtctcggctgacaaggcttcgccctttgatgcaagcagcctgcccagagaatcctctgcaatttccaaggctgcaacaaacaattctctcctcgccaatcccgggccacaccgcacgctcaactcaccgacaaggctatcgctccgcagccacgccaagtccataacaacaaacagcggccgctcccctccagaaaaagaggcagggattaaggccggcgagatgccatcacggcattgtgctcctctcggtaggctgaaccctccctccccagccccggacacactatccgcttatgcacaatggaggaggcacacttggcgaggcgctgtccagggattcgcgcatgccgtggtactttttgtgttggcgacgagagtagctagtagatgggcgcgctgtgcgatgttaccttgaataataaactaaaaaggggggtgctagatctccttgtacaagaacatgatgacttgatcgattgatgaaggaagtgaattcgacgcgatctccatcgaagcttgaggcatcagggacgatgacatctagggagccaccgccgaggaagaaccaccgagagagtttcaagcacccagtctacttggggcacgatatgtacacattagaaatccccccagcctctcgcttctaattccttgtgctgcctcctctcctgtcttctcttcttcctctttagttttccttgtcacgatcagaagcctctctctctctctcattctctttcttggagtgaagcagatctgaagtattctctcatgttcgtcagacatgagcaccagagctttctacagtatcatcttgtgagagtacctcggccagagagttttcatattgaagcttggcccaccattttcgagcagtcattggcgcttccgttcccttccaattgaggcctctgattgtcttgatataaaagcacattcgcaacttttgtttttgataggtgagcattcagtggaagaagcggctccaggagcaaatgaagaacgaggcggcggaggaggcggcctctcctcgcatggcggttagttgaaggcgataatagcgtgagggaagagaccaacttggcttgttcttttcaaaggacgttgactgtaacctcctagatgtctttcatgaagtgaccagaatgctgtcgccacatggggtcgaaggcatcgggaggagcaaactaaaaagcgcctgggccttcaagactctctaaagagttagagtcctaagaaaagtccgacgaggtagatgttggggtgtcaacgccgctggcgggcagcatggtgagcaagattgacatattccatatcttaatagtaccatcgtctgaaccagatgctacacgagtcgagtcgtgcgacaaggccaccgaagaaaccttcgtactgtgaccctcaagcacccgtaggcattgc

SEQ ID NO: 37 - DNA, 2606 bp, synthetic construct
  misc_feature 1..2606: note = Synthetic donor-DNA.
  source 1..2606: mol_type = other DNA
ttatggggtggtatcgcatgttatcttcgtcccattgcaacgacctacatacatactccttttccggtattacccccactatgcattgatggcgttactaagctttctggttgcgttagcttgataaaacattcccacgtacgaagctacctcgagatatgtgaagctgagtggtgctctattagtcgagggaagatgaaatcatccagtcatatatcatcataggtatgaagcgcccatacgggtaaggtgccgctactacgcgagcaagtggctgcagcaatgccttgggagacgaatcggagttgatgttgaccctctcttccagtaagtaataaagcaaatcaggaagctacctacgcttcacccacgtaccttatgcaaagcaactgatctgggttaaagaaaggcttatacatagctcagcgctccggtagtggatgagcttgctaccaattgcgtgaaaattgcgaaagagcatgtctcgatcccaatgcttgatgaatgacttagccgtacgtgtaagagctttatagaaagtagatagccccgtaacctgtgtacacatcgagcagcgtcccgcaaccaggtcatgttgaagatgttacatatctcttgaaccaggtagaagtgacatgtcagctacgagacctggtgactattgagaaaacgccaacaaacgcaagcgctgagtttgctcaacaacgtgagaccaaagttagtttacgtagtttacgtagctcacaaggctcattcagcctctagtccgatattcctagacgtctgcaaagcttcagctactgaggacaccattggctgtcttcttctcggagatgttcttcgcaataggcaggtcgcagagcaagacatgctcaaccctctcaacagttattcctttttctcaatggtgccttccttctggttcgaccggcatctaatgacgtgtagatatggctaagatatcttcaagcttatgcgaaaagtgtctcccgcttcccaggaaaatcctacattcctatttcgatcaccacctccgattgcatcttcaagcccttgttttcagcaatgagaaccattacaagacgaaccggagcagtgtacacccatagaattgtaagtatataaagaccctctcggaacctccttcaactcatacctatacaggagcaacagctgttcgccccacagtccacgatcgaccaacatcttcatccaaaggctttgcccgtgtcaactgtctcacttccacccatctcaactgcacatcttcatccgaaaccatcaagatgaagaccctcgctaataaactgttcgtcgatgagggagatactgaggagcgcctgctcagatctagaattcatcgggagcaacaacatgaggttcgaactagaatatgagaccaagtgcacgcgaccatagcaatgaagtacaacacgaaatatatgtacttgagcactgaagcattacaatagaaaataagtgatctgtcattgttgaatatcttatatactatgtgctgtcagacggagcatttgactgcaaaattgttggttttgcattgggcatttttccatggcaatagcaatagctaaaactcagcctcccacgtctctgctacttatactcatacaaagctatttattgtatagcaacgctgtatcaagaagcatgtagtactaatagggtacagtttgtgtcatgtcattatatcactgaattgggagatggagcagatatacgcaccacaaaggtcactcatttttcagttttccaggattgattgtcgtgcctatctataaaacttgtttcgatggtattcttattttagatgcagcttgacaacatacataatacagtgaatgacggatttactacgggaatcatggctcggagcctcactgactgatgacgaggctctcacagctagagaagggtaggtcatacgtctctgaatcacgatgaatatccagaaataatcgcttcctatgaattgcacaactccataggtagaggtgctcatggggatttgttactgagcacgatataagctggacgaccacgtgctaaagctcgggccattcgtcgttgatataattgctctgcgtataccaggagcagattcttcgcagattacttgatataagtactgctctagttacgcccacaaaggcgagagataagcatacggcagaagcttgcaatctacgacatcattgagcttggaacaacgcaatgagtagaattactttgactcaagtattgggcggtatggtaggcattggatgtcgtaagttacacatactcttttaccaatgaagacagcgggtactagcctacatacttttgagtgagtcacaaatgagggtgttgtatgaagcattcgccatcaaaagctggacatgcgttgtggctaatgaagtagaaaagggggtctgctggcgtgtcctgtggtttgcttgttatgcgggactcaacaatggacgcaacgtcaacttattctatgagcgaatagctctgacatgatgctccagtgcttgaggcagacaaagcaagtcagcaatgacaagggcttatgattggtgaatgatacatcagccttaatgcgc

SEQ ID NO: 38 - DNA, 2406 bp, synthetic construct
  misc_feature 1..2406: note = Synthetic donor-DNA.
  source 1..2406: mol_type = other DNA
cctgttcatgcccggtcaccaggccgctctgattgctgggacggcggatttaacatagactaacggattagacttaattcggccgccatgttgttgatccctagtcgattgggctagtagttcttctttgctcagtcccctatgcgagccaggagcacgaggcagatccgagtcaagcaaggcatggatgggatgggatggctctcgtggctttgaattaccgttgatgcgagagagagagagagattgggccatacgaggcctgattcgtgcttgcggctggtgactggccgtctgggggcccgagcatgccactggcagtgggatgcggtggaacacgattgcaacattgcccccgaaaggatgggctggtaacttcgtctgttcaggtcgacctcggtcatacaaagcaaagacgcgggcgcagcgccattgcccaagtcatgtcggcccgcctaccccagcctgtcatatcgtcacacggactgaactgtgggctgctgtttaagcgttgagcaggaagacccttgatagacaaagatcggcaatggatatctctctcgaggcctgcttatttccctggcctccgcagtgtcgacattgcacggccagccacgactcgggattggccggccataggtagtggcgaggacgaatggtcaaaggactatgtatcatctcgacttatgcgcctcgggagacaagtcacactgttgcgccgtctccgagcgaatggccgtggccatgggctagcaggactctggacttctatcttgggatgcttcacgatacgtgatcgacaaaggttccagcgcctccattggagcaaggcagaaatggcagtacgctcctcggccgacctctcttggtgggttaggagaccaggcacgccctccacatggatgccacccctggacgcggacgcttcattcgctcgcgggccggtaacgacgatctacagccatctggcctgattgcggaggtcaattccaccatctcgactcctcctcccatcaccatgccccagcaagtgtgtgaagctcggggaggatggcgcaaaccgaccttcctaaaccatgctcttgaggccgtcaacaacgacaatgccccggagatggcagattatcgacgaagcgtttgtcgatggagctcaaggggatatataaagacgtccggcgtccctccgtctgctgttggagctaataagtctggctctcaagacttgtaaatactcttggtgtatatagcaagatgtcagcacatagcagagcaatgacgaaactgtaatgaaggacgctttgggtctcagtgacttgtgaaatgaggtcccttcctacctatccatcgtggtccgtaggggtaaggtgcgaagtctacttgcaatgcttgaactgagcctcggaatgatacaattatccatggcgattgtgaatactgagatgaggtctcgttgatgatgtgcttagcaatatcaacattaaatcctgctttgccccatcctgaccttgatctccaagccactgacaaataggtgttcatgcactatcatgggttacttgatgtatcgctccggatatttagctgcatcactgttaatgattaaatccaagtcctgtgttatacattaaagaaggtccagagtggtggttctgcccggcttcggagatttgcaaggtctcgagcgagtccgaagctgggctgaagcagcgcagggcctgactctgggtatgtgttgatgtagtggcggacgatgccatgaacatggactttttcttttctcttacccctgtgatgctctttgtcatggcgagaacgtatgctgagaacagttgatatgagtgagattgtttcgaggagggacatcaagggcttgatgtctttggtcaccagcctttctgcttctaactcggaatcatgaaaaaaccgacgtcttgctgacagatatcgccctggggcctagaatatacagaaggatagttctgactgtgacagccagagaagtggtagggctgtaagggggtacgagatgagctgtgctcgataggtgagctggaatgctatttgatggatgtacagcgagtgttggggtggttttgcagctgtaaataagggaattgacaggggaggcgatgaacacgccggcaaggaaagcgtctcatgctatgcatggggattttgaacaagaagagtgcagaatttactcctgtgaagaaggggagtgatgagaagcgatataggggatgattgacgagggagttgatgattgagagggttcttacatagtgcggctctttgatgactttgcagagtgtctatttagattgtgttatttatggtttctgctgcgtttgtgattagttgatgcatgtatagcgtcaaaagctctttttacgctgccatgccatc

SEQ ID NO: 39 - DNA, 2490 bp, synthetic construct
  misc_feature 1..2490: note = Synthetic donor-DNA.
  source 1..2490: mol_type = other DNA
gtgagcatatcagctggtactacttgaaatatcacctctatcaggagcatacgatacaagacttctactatacaatcacctgagaggcttccagtcgcaagcgccagtcaatctgattgtaatatggcggactagacacgagtatgcaagcagaagcatcttcctcgttcgtcttgatcgtcactataagtcgcaatatacagtctaattctagaggccagtgtgacctcatgagggactatgggaaaacaaggaagagataccgcgacatgacgggaggtacttggcggacttgaggcaaataacatgcgcgccagtagtgacatcacaagcccaggcgattgcctctgacttatggaagatgatagaaagaaagagaccagcctcgtcttttcctcaccaagaagacaaggcagtgtgaagtaatcccactcgagcaggtaactacgatgtctgccgctttccctctcagaacgtggagaaatcggaatatcggcatcgaacctagtagatgcctgagcaaactagccggggtcggtactttgtaagagcaaatcaggaaggtgattagtccccgatagttactgagatcaggcaagatcgccaaggcgcgaatccgatgaagaatgcttaaacattaggttgtctctcccccagtccggtaataaccataatgatccgcaataccattgtgtgtgttgcgagattctgctgtaactcgtgaatcaaagaatctccagactcctcatatccgtagcttctcattctttatctcgtagtacctctagccaggaaccagcagtatcactgcactgaagttgcacggcatctcggcacgtcgatgcggccagcaattcctccggtgctactccccacccacgtctggtccgtagggtgtggtagcatgtatcgcccaatttagtttgtctgctgcgggaaggacttatgttgcagcgaatcccatgtgaagcattgcattggcctcctcacagtggcgtaggcgccatgttatcacgaacctcaacacgcgaatacggcttaatgaggaatctggatgttgacgccagctcggagatgtcattcaagcaggaaggtgtcttgagaaagtctgcaaatggctcgtggacctaagatcattggcatcttaagatgtgagtagttggggaagggatctcgattgctctcaagtacttcagttataaatattaccctctgcctcttcttgagttgatcttggtttaaatcagacttaataacaggacctttggccgctctatctggcagttctcccggcaacatgtagcagaagtatcatgtgccacaaatttttgagtctatacatactgatgatacaaatgaatacgaaacttttcattatcaaatcctcaagtattaaagtaatgtgactggcgatgaagagaggacgtggctgttggcaagggcgtgtgcaatattatattgtacagaatacatgtcgcaaagctatacaggcgttctattaccattaggacgttgtctgaagaaagtatacaggcctccttctgccatacctgcaatctgcataataataggatgcagaagatataagatcatagcgtaaaggggtgctatcaaatgtccatttcaaatgtaacaattcagcctccccgagggacgccactggccttgcgggcagtgaaaattctgaatgagaaatactcacggcgactgttgctcggcaaaacctttactgtgattccattcattattcgtaagtttactatgtggcttgcataagccacagtgatggcgacataagcacaactgctctaaaccaatagctcataacatattctagcatgactgactgactactttgagccaagctgatgttgtactgccttctactcaacttggctcgctttgaatcaaacattgcttacgcggggtatatgaaattcgcatctttcttccgtaggagccgataaaattcccatccccaacaaattgaaagctggtatgccatttcatatcgcagtggcccctttacttaagagcgactataatgtgtgccaagtttgagagtgtgaaattggagcagcaacttatggtatattaacccatgttaggagcgtgaggggttagaaagtactattggcacaacatggcatggaataatggcgatggtttgtataactcctgataaaaactcagctttggagtgaagcgctccgtatgttagccagggctgagacattctctacagagagacctgctcttgccttgctgttaatctttccttctatttcttttcatacaaatgtgctgggatggcacatctcacaataaattcaaatggtgtattttctggcaggcagtctgcttccccagaaccggggcggcaatggctatatcaaatgtagagtccatacatggggcgcctgcgctcaatcacaacatagctgtcgaagtgtcaaacaaaatctcaccagccagctccttgggccttcg

SEQ ID NO: 40 - DNA, 28 bp, synthetic construct
  source 1..28: mol_type = other DNA
aaatattaccctctgcctcttcttgagt

SEQ ID NO: 41 - DNA, 18 bp, synthetic construct
  source 1..18: mol_type = other DNA
ctgctacatgttgccggg

SEQ ID NO: 42 - DNA, 22 bp, synthetic construct
  source 1..22: mol_type = other DNA
tgtgagtagttggggaagggat

SEQ ID NO: 43 - DNA, 38 bp, synthetic construct
  source 1..38: mol_type = other DNA
tttgtatcatcagtatgtatagactcaaaaatttgtgg

SEQ ID NO: 44 - DNA, 18 bp, synthetic construct
  source 1..18: mol_type = other DNA
ctggatgttgacgccagc

SEQ ID NO: 45 - DNA, 22 bp, synthetic construct
  source 1..22: mol_type = other DNA
taatattgcacacgcccttgcc

SEQ ID NO: 46 - DNA, 27 bp, synthetic construct
  source 1..27: mol_type = other DNA
tagcatgtatcgcccaatttagtttgt

SEQ ID NO: 47 - DNA, 30 bp, synthetic construct
  source 1..30: mol_type = other DNA
acccctttacgctatgatcttatatcttct

SEQ ID NO: 48 - DNA, 26 bp, synthetic construct
  source 1..26: mol_type = other DNA
gcaaatcaggaaggtgattagtcccc

SEQ ID NO: 49 - DNA, 31 bp, synthetic construct
  source 1..31: mol_type = other DNA
tacggaagaaagatgcgaatttcatataccc

SEQ ID NO: 50 - DNA, 17 bp, synthetic construct
  source 1..17: mol_type = other DNA
agaacagcagcagcgct

SEQ ID NO: 51 - DNA, 29 bp, synthetic construct
  source 1..29: mol_type = other DNA
cgccattagatgttttatacctgcttaca

SEQ ID NO: 52 - DNA, 15 bp, synthetic construct
  source 1..15: mol_type = other DNA
actgggcagcctccc

SEQ ID NO: 53 - DNA, 21 bp, synthetic construct
  source 1..21: mol_type = other DNA
cttcctgcaatgagcagctca

SEQ ID NO: 54 - DNA, 21 bp, synthetic construct
  source 1..21: mol_type = other DNA
cagcgaagatggtactcccct

SEQ ID NO: 55 - DNA, 21 bp, synthetic construct
  source 1..21: mol_type = other DNA
gcgatggattttggagacggt

SEQ ID NO: 56 - DNA, 22 bp, synthetic construct
  source 1..22: mol_type = other DNA
ctagacatgggtcgaaagtggc

SEQ ID NO: 57 - DNA, 18 bp, synthetic construct
  source 1..18: mol_type = other DNA
caagatcggtgggggtgc

SEQ ID NO: 58 - DNA, 17 bp, synthetic construct
  source 1..17: mol_type = other DNA
aacgttccgcatcggcc

SEQ ID NO: 59 - DNA, 17 bp, synthetic construct
  source 1..17: mol_type = other DNA
cgagggggcagagttgc